(19) United States
(12) Patent Application Publication
(10) Pub. No.: US 2025/0129132 A1
(43) Pub. Date: Apr. 24, 2025

(54) GENETICALLY ENCODED AND EXOGENOUSLY TRIGGERED PROTEIN-PROTEIN LIGATION

(71) Applicant: University of Washington, Seattle, WA (US)

(72) Inventors: Cole Alexander DeForest, Seattle, WA (US); Emily R. Ruskowitz, Seattle, WA (US)

(73) Assignee: University of Washington, Seattle, WA (US)

(21) Appl. No.: 18/693,450

(22) PCT Filed: Sep. 21, 2022
(86) PCT No.: PCT/US2022/076816

§ 371 Date: Mar. 19, 2024

Related U.S. Application Data
(60) Provisional application No. 63/246,749, filed on Sep. 21, 2021.

(51) Int. Cl. C07K 14/46; C12N 9/02; C12N 9/12

(52) U.S. Cl. CPC C07K 14/461; C12N 9/0069; C12N 9/1241; C12Y 113/12013; C07K 2319/00

(57) ABSTRACT

Described herein is a generalizable strategy to rapidly and irreversibly activate protein function with full spatiotemporal control. Through development of an exogenously triggerable self-assembling protein construct, bioactive proteins can be stably reassembled from non-functional split fragment pairs following exposure to a stimulus (e.g., light).

### CROSS-REFERENCE TO RELATED APPLICATION

This application claims the benefit of U.S. Application No. 63/246,749, filed on Sep. 21, 2021, the disclosure of which is hereby incorporated by reference in its entirety.

### STATEMENT OF GOVERNMENT LICENSE RIGHTS

This invention was made with government support under Grant No. R35 GM138036, awarded by the National Institutes of Health (NIH) and Grant Nos. CBET 1803054 and DMR 1652141 and DMR 1807398, awarded by the National Science Foundation (NSF). The government has certain rights in the invention.

### STATEMENT REGARDING SEQUENCE LISTING

The sequence listing associated with this application is provided in xml format in lieu of a paper copy and is hereby incorporated by reference into the specification. The name of the text file containing the sequence listing is 3915-P1226WOUW_Seq_List_20220921_ST26.xml. The xml file is 69 KB: was created on Sep. 21, 2022; and is being submitted via EFS-Web with the filing of the specification.

### BACKGROUND

Biology is comprised of a series of well-orchestrated chemical reactions, precisely controlled in time and space. Proteins act as the conductors of these reactions through interactions with other biomolecules, e.g., small molecules, DNA, and proteins. Protein-protein interactions (PPIs) represent the primary mechanism of cellular regulation, dictating complex biological processes including cell growth and migration, cell-matrix interactions, and diseases including Alzheimer's. Since PPI govern nearly all cellular activities, there is tremendous interest to precisely control when and where such reactions occur. However, precisely controlling when and where such reactions occur is difficult to achieve as the biological environment is characterized by fluctuating solution conditions from varying pH, presence of reductive/oxidative species, and a staggeringly complex milieu of functional reactive groups. Performing additive chemistry in this myriad of chemical environments requires highly specific and “unsensitive” reactions that do not rely on damaging/cross-reactive species as, for example, radical-based chemistries.

Spontaneous ligation chemistries which utilize bioorthogonal functionalities can be used to control biologic function by forcing specific PPIs to occur. Complementary reactive handles (e.g., azide/alkyne, aldehyde/ketone) are typically introduced exogenously with little specificity (e.g., NHS-chemistry) or metabolically with single-residue or single-site precision. Chemistries introduced exogenously with little specificity or metabolically with single-residue or single-site precision is a problem with current state of the art since many cellular processes require single site precision. Perfect amino acid specificity is potentially achieved through genetic code expansion, a technique that relies on unnatural tRNA/tRNA synthetase (tRNArs) pairs engineered to insert a non-canonical amino acid at amber stop codon sites (TAG). By overriding this rarely used codon, a bioorthogonal reactive moiety can be site-specifically incorporated during protein translation avoiding further modification. Installing reactive groups that do not naturally exist in living systems affords high reaction specificity.

More recently, spontaneous ligation of naturally derived reactive protein partners presented a genetically encoded means to achieve precise control over PPIs. SpyCatcher (SC), SnoopCatcher and SdyCatcher ligations, proceeding through isopeptide covalent bond formation between split-protein fragments upon re-association, leads to long-term binding. Since these strategies are bioorthogonal, fast, and genetically encodable, there have been an explosion of applications in the few short years since their development including intracellular protein localization, biomaterial functionalization, and modular vaccine development. Though spontaneous ligation strategies afford reaction specificity, tremendous benefit comes from being able to externally trigger when and where biological reactions occur. PPIs can be temporally controlled through exogenous addition of small molecule. Small molecule triggering biological reactions is part of the current state of the art. The problem with small molecule triggering biological reactions is that the small molecule is not localized at the location where it is needed and, as such, can lead to cellular impairment, unwanted reactions and waste of product. Spatially selective activation when small molecules are used requires localization of the stimuli to a region of interest like local heating, sonication, or light. Unlike other triggers, light is unique in that it can be controlled in both time and space without disrupting cellular function, offering specification to when, where, and to what extent reaction occurs. Naturally occurring photoresponsive protein systems (e.g., Magnets, PhyB-PIF, Dropna) have been developed in recent years by the optogenetic community as a handle for triggering PPI in individual cells. These photoresponsive proteins are a genetically encodable approach to achieving spatial control of PPIs by photoactivating protein multimerization. Though such reactions have found great utility in optogenetic regulation of intracellular signaling, reactions are non-covalent and reversible, placing limitations on their potential applications.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In the presented application, we overcome these problems with the current state of the art by developing a versatile protein-protein binding reaction scheme that is (1) highly specific, (2) genetically encoded, (3) phototriggered, and (4) irreversible. Developing a reaction scheme that is highly specific, genetically encoded, phototriggered and irreversible solves many of the problems encountered with the current state of the art. High reaction specificity permits reactivity within a biological context without concern of cross-reactions leading to cellular impairment. Constraining the scheme to genetically encoded chemistries enables scalable synthesis of reactive components through conventional fermentation processes. Further, reactions can be performed in vitro and in vivo by expressing components within the system in study. Light-activated chemistries afford cytocompatible and spatiotemporal control over the extent of interaction by manipulating dosages. Finally, reaction irreversibility ensures long-term, stable interactions independent of solution conditions.

Presented is a technology that solves the problems with the use of current state of the art. Current state of the art technologies lack single site precision with bioorthogonality and irreversibility. Presented is a system of an amino acid, a photolabile cage or stimulus responsive cage, and an irreversible conjugation of recombinant proteins; together these give Spatiotemporal Functional Assembly of Split Protein Pairs through a Light-Activated SpyLigation or LASL. The presented technologies impart single site precision (spatiotemporal control) with bioorthogonality and irreversibility and are particularly useful where these positive features are required properties to reaction sequences. The single site precision with bioorthogonality and irreversibility technology involves the combination of protecting caging groups with amino acids and proteins. Caging groups that are photolabile and/or stimuli responsive are applied to amino acids. The cage protected amino acids are then applied to irreversible conjugation of recombinant protein molecules. This system which constitutes the core of LASL is then applied to larger protein fragments. The protein molecules are exposed to energy when a reaction is needed. The energy can be directed to a specific area and time for spatiotemporal control. This combination allows spatiotemporal control of reactions. The technology is useful in the fields of drugs, drug delivery, therapeutics and DNA recombination.

In one aspect, an exogenously triggerable self-assembling protein construct is provided, comprising:
a caged reactive first protein fragment comprising a first stimulus-responsive cleavable moiety capable of cleaving from the caged reactive first protein fragment, upon application of a predetermined first stimulus, to provide a reactive first protein fragment;
a first split protein linked with the caged reactive first protein fragment;
a complementary reactive second protein fragment capable of reacting with the first reactive protein fragment; and
a second split protein linked with the complementary reactive second protein fragment,
wherein the first reactive protein fragment is adapted to react covalently with the complementary reactive second protein fragment to provide a self-assembled ligated protein or a portion thereof; and
wherein the first split protein is adapted to associate with the second split protein and to form an active protein in accordance with the reaction of the first reactive protein fragment and the complementary reactive second protein fragment providing the self-assembled protein or the portion thereof.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1A-1F: Light-activated SpyLigation (LASL) affords complete spatiotemporal control over protein activation within living systems FIG. 1A: A photocaged lysine is site-specifically incorporated within the active site of SpyCatcher (SC) during protein translation via an unnatural tRNA/tRNA synthetase pair, giving photoactivatable SC (pSC, SEQ ID No. 26). FIG. 1B: Owing to the bulky photocage masking its reactive amine, pSC (SEQ ID No. 26) remains inactive and unable to interact with or covalently bind SpyTag ((SEQ ID No. 7, SEQ ID No. 8), ST). FIG. 1C: With user-directed light exposure, the lysine is liberated to generate newly uncaged SC (SEQ ID No. 25), capable of spontaneous isopeptide bond formation with ST (SEQ ID No. 7, SEQ ID No. 8). FIG. 1D: Photoactivation is imparted through an ortho-nitrobenzyloxycarbonyl (oNB) moiety installed on the ε-amine of lysine, such that light exposure restores the native residue. FIG. 1E: When pSC (SEQ ID No. 26) and ST are genetically fused to otherwise non-associative split proteins, irreversible protein activation is photochemically regulated. FIG. 1F: LASL of the SC (SEQ ID No. 25)/ST (SEQ ID No. 7, SEQ ID No. 8) pairs to irreversibly activate split proteins offers many distinct advantages over existing optogenetic strategies and can be used to interrogate a variety of biological functions in 4D.

FIG. 2A-2C: Photoactivatable SpyCatcher (pSC, SEQ ID No. 26) provides user-control of SpyLigation in solution. FIG. 2A: Intact protein mass spectrometry of SC (SEQ ID No. 25), pSC (SEQ ID No. 26), and light-treated pSC (SEQ ID No. 26, +hv. λ=365 nm, 20 mW cm², 30 min). Dashed lines indicate expected masses of SC (SEQ ID No. 25) and pSC (SEQ ID No. 26) containing a single Lys(oNB) (red star). FIG. 2B: Covalent linkage of pSC (SEQ ID No. 26, \textcircled{1}) and a Protein-Of-Interest (POI)-SpyTag fusion (GST-ST (SEQ ID No. 28), \textcircled{2} is inhibited by the presence of Lys(oNB) at a critical lysine residue, pSC (SEQ ID No. 26) photoactivation allows for formation of the covalently ligated product (\textcircled{3}). FIG. 2C: Extent of LASL between pSC (SEQ ID No. 26) and GST-ST (SEQ ID No. 28, 10 μM, 37° C.) varies with light exposure (λ=365 nm. 10 mW cm⁻², 0-90 min), as visualized by SDS-PAGE following 18 h of post-light incubation. FIG. 2D: pSC (SEQ ID No. 26) photoactivation and ligated product formation exhibits a light dose-dependency, as determined by SDS-PAGE band intensity quantification for each species as labeled in c. Light dosage is calculated as the product of light intensity and exposure time. Error bars correspond to the S.D. about the mean for 4 experimental replicates. FIG. 2E: Reactivity of pSC (SEQ ID No. 26) and GST-ST (SEQ ID No. 28, 10 μM, 37° C., 0.5 h) with and without light treatment (20 mW cm⁻², λ=365 nm, 0.75 h) in E coli lysate analyzed by SDS-PAGE.

FIGS. 3A-3I: LASL enables site-specific patterned protein localization in 3D biomaterials and living cells. FIG. 3A: Step-growth polymerization of PEG-tetraBCN and PEG-diazide in the presence of pSC-N₃ (SEQ ID No. 26) form a SPAAC-based hydrogel uniformly decorated with the photocaged SpyCatcher. Photoactivation permits spatiotemporally defined immobilization of POI-SpyTag (SEQ ID No. 7. SEQ ID No. 8) constructs via LASL. FIG. 3B: Mask-based photolithographic exposure (λ=365 nm, 20 mW cm⁻², 15 min) generated discrete patterns of immobilized mRuby-ST (SEQ ID No. 29) throughout the gel thickness. FIG. 3C-FIG. 3E: By treating gels with linear gradients of light [created by covering samples with an opaque photomask that moves from right-to-left in relation to the sample shown at rates of 0.13 (light gray), 0.2 (dark gray), and 0.4 (black) mm min⁻¹], exponential gradients of mRuby-ST (SEQ ID No. 29) were generated in a dose-dependent manner. Relative protein concentrations were determined by sample fluorescence across the length of the gels. Solid lines depict predicted concentrations based on pSC (SEQ ID No. 26) activation kinetics. FIG. 3F-3I: Optogenetic specification of protein membrane tethering in mammalian cells. FIG. 3F: Schematic of gene cassette used to prime cells for LASL-mediated plasma membrane labeling, where a CAAX-anchored EGFP-ST is covalently ligated with cytosolic pSC-mCh upon light exposure. FIG. 3G: Representative fluorescent micrographs of transfected HEK-293T cells imaged 6 h following treatment with varied light exposures (λ=365 nm, 20 mW cm⁻², 0-20 min). FIG. 3H: Membrane labeling with mCh scales in a statistically significant manner with light exposure, relative fluorescence visualized as violin scatter plots. FIG. 3I: Intracellular mCh distribution transitions through LASL from uniformly cytosolic to more membrane localized with increased light treatment durations. Asterisks denote conditions with statistically significant differences in signal (p<0.05, unpaired t-tests). Scale bars, 250 μm (FIG. 3B, FIG. 3E), 20 μm (FIG. 3F).

FIG. 4A-4K: Assembly of UnaG (SEQ ID No. 41) through LASL of split protein fragments in solution. FIG. 4A: UnaG (SEQ ID No. 41) is split into N-(nUnaG) and C-terminal (cUnaG) fragments each genetically fused to ST (SEQ ID No. 7, SEQ ID No. 8) and pSC (SEQ ID No. 26). Fragments remain inactive until photoactivation of pSC (SEQ ID No. 26) and LASL-mediated functional assembly of split fragments to restore UnaG (SEQ ID No. 41) fluorescence. FIG. 4B: All possible fusion variants of ST/SC and UnaG (SEQ ID No. 35, SEQ ID No. 34) fragments were cloned and recombinantly expressed to screen for maximum assembly fluorescence. FIG. 4C Individual and combined variant (10 μM) fluorescence after reaction for 0.5 (outline boxes) and 24 FIG. 4H (black circles). FIG. 4D: Excitation (dashed lines) and emission (solid lines) spectra of pSC-cUnaG (SEQ ID No. 39) kept in the dark (−hv) or exposed to light (+hv, λ=365 nm. 20 mW cm⁻², 20 mm) and reacted with nUnaG-ST (SEQ ID No. 31, \textcircled{1}). Photoactivated pSC-cUnaG (SEQ ID No. 39) exhibited spectra similar to its wildtype counterpart (SC-cUnaG (SEQ ID No. 37), \textcircled{7}) when ligated to nUnaG-ST (SEQ ID No. 31). FIG. 4E: UnaG reconstitution and accompanying fluorescence from nUnaG-ST (SEQ ID No. 31) and light-treated pSC-cUnaG (SEQ ID No. 39) exhibited dose-dependency. FIG. 4F: UnaG can be spatiotemporally reassembled within hydrogel biomaterials functionalized with pSC-cUnaG (SEQ ID No. 39) and patterned with nUnaG-ST (SEQ ID No. 31) via LASL. FIG. 4G: Mask-based photolithographic exposure generated discrete patterns of active UnaG (SEQ ID No. 41) throughout the gel thickness. FIG. 4H: Multiphoton laser-scanning lithography affords patterned protein activation with full 3D control. FIG. 4I-FIG. 4K, Split NanoLuc is reassembled via LASL in a light dose-dependent manner mirroring kinetic results for UnaG (SEQ ID No. 41). Data are mean±1 S.D. normalized to the experimental minimum/maximum (n=3 experimental replicates). Error bars in FIG. 4K are substantially smaller than symbols indicating mean luminescence. Scale bars, 500 μm (FIG. 4G), 50 μm (FIG. 4H).

FIG. 5A-5I: Photoactivation of split UnaG (SEQ ID No. 41) with spatiotemporal precision in living cells through intracellular LASL. FIG. 5A. Schematic of pSC-UnaG gene cassette used to prime cells for LASL of non-associative UnaG (SEQ ID No. 41) fragments. FIG. 5B: Representative fluorescent images of transfected HEK-293T cells (red) with (+hv) and without (−hv) light illustrate UnaG (SEQ ID No. 41) photoactivation (light grey/white) by LASL. FIG. 5C: Timelapse of intracellular UnaG (SEQ ID No. 41) photoactivation after light treatment (+hv), normalized to initial UnaG (SEQ ID No. 41)/mCh ratios. FIG. 5D-FIG. 5F: Mask-based photolithography spatiotemporally directs UnaG (SEQ ID No. 41) reassembly within HEK-293T cell culture. FIG. 5D: Fluorescent images of culture dish with inlays of exposure boundary magnified. FIG. 5E: Individual cell UnaG (SEQ ID No. 41)/mCh signal quantified radially outwards from the photomask's center, normalized to the average UnaG (SEQ ID No. 41)/mCh ratio in unexposed cells. Dashed line indicates exposure edge. FIG. 5F: Violin scatter plots of normalized UnaG/mCh ratios in light-(un)exposed regions. FIG. 5G-FIG. 5I, Spatially vaned light exposure (0-10 mm) yielded dose-dependent UnaG (SEQ ID No. 41) activation throughout mammalian culture. FIG. 5G: Fluorescent images of culture dish with dashed lines highlighting exposure pattern. FIG. 5H: Individual cell UnaG (SEQ ID No. 41)/mCh signal quantified for each exposure subregion, normalized to average unexposed UnaG (SEQ ID No. 41)/mCh ratio. i, Individual cell mCh signal quantified for each exposure subregion. Light treatments, 20 mW cm⁻², λ=365 nm (FIG. 5B-FIG. 5F). Asterisks denote conditions with statistically significant differences in signal (p<0.0001, unpaired t-tests). Scale bars, 20 μm (FIG. 5B), 1 mm (FIG. 5D, FIGURE SG).

FIG. 6A-6D: Spatially controlled photoactivation of primary cell genome editing via LASL. FIG. 6A: Cre recombinase split into inactive N—(nCre) and C-terminal (cCre) fragments and respectively genetically fused to ST (SEQ ID No. 7, SEQ ID No. 8) and pSC (SEQ ID No 26) can be functionally reassembled using LASL. FIG. 6B: Transgenic mouse dermal fibroblasts contain a dual-color reporter for Cre activity; site-specific recombination of DNA between loxP sites results in tdTomato gene excision and expression of a downstream EGFP. FIG. 6C: Cells transfected with the pSC-Cre gene cassette and stimulated with light (Q=365 nm, 20 mW cm², 3 min) exhibit an irreversible red (darker grey)-to-green fluorescence (lighter grey) switch associated with Cre-mediated genome editing. FIG. 6D: Using a circle photomask (2 mm diameter opening), recombination can be spatially regulated via photolithographically controlled LASL. Fluorescent image of patterned cells with inlay of subset of exposed region magnified. Scale bars, 50 ™ (FIG. 6C), 1 mm (FIG. 6D).

FIG. 7A-7B: Photouncaging of Lys(oNB) in solution. FIG. 7 A: Lys(oNB) (0.1 mg mL⁻¹ in phosphate-buffered saline, PBS. pH=7.4) was exposed to near-UV light (λ=365 nm, 10 mW cm⁻²) for varying times (0-20 mm). FIG. 7B: Photouncaging was tracked through UV-Vis absorption spectrometry. Arrow indicates direction of absorbance shifts throughout exposure. FIG. 7C: Absorbance measurements at λ=365 nm were used to determine the first-order degradation constant as 0.16±0.02 min⁻¹. We note this to be ˜3× faster than what was observed for pSC (SEQ ID No. 26) uncaging, attributed to differences in the local chemical environment in solution and within the protein.

FIG. 8: Assessing purity of expressed pSC (SEQ ID No. 26) through SDS-PAGE analysis. Expression and purification of pSC (SEQ ID No. 26) in the presence and absence of Lys(oNB) was tracked using SDS-PAGE with proteins, as visualized through Coomassie staining. Lanes correspond to:
\textcircled{1} Full cell lysate
\textcircled{2} Soluble fraction
\textcircled{3} Column flow-through
\textcircled{4} Final wash
\textcircled{5} Elution
\textcircled{6} Concentrated protein
SC=pure SC protein

The purified and concentrated pSC (SEQ ID No. 26, circled) aligns with recombinant SC (band in right-most lane). Lys(oNB) is for proper amber suppression and expression of pSC (SEQ ID No. 26), indicated by the absence of an appropriately sized protein band in the purification lacking Lys(oNB). This confirms the orthogonality of mutant tRNA/tRNA synthetase pair used for incorporation.

FIG. 9A-9B. LASL proceeds efficiently with premixed pSC (SEQ ID No. 26) and ST (SEQ ID No. 7, SEQ ID No. 8) species. FIG. 9A: Purified pSC (SEQ ID No. 26) protein (20 μM) was mixed with GST-ST (SEQ ID No. 28, 25 μM) prior to treatment with varied light dosages (λ=365 nm, 10 mW cm⁻², 0-90 min) and reacted to a non-kinetically limited endpoint (18 h, 37° C.) prior to analysis. Samples were diluted 1:1 with 2× Laemmli sample buffer and briefly boiled before analysis by SDS-PAGE (8-16% TGX precast mini-gel, BioRad). FIG. 9B: Experimental replicates (n=4) were quantified by determining the band intensity of the two reactants (pSC (SEQ ID No. 26) and GST-ST (SEQ ID No. 28)) and the LASL product at each time point in FIJI. pSC (SEQ ID No. 26) band is marked with \textcircled{1} and triangle markers, GST-ST (SEQ ID No. 28) with \textcircled{2} square markers, and the covalent ligated product with \textcircled{3} circle markers. Light dosage is calculated as the product of light intensity and exposure time. Error bars correspond to the S.D. about the mean for 4 experimental replicates.

FIG. 10A-10C: GST remains bioactive following LASL. FIG. 10A: We employed a 1-chloro-2,4-dinitrobenzene (CDNB) colorimetric assay to assess GST activity before and after LASL. FIG. 10B: Samples of pSC (SEQ ID No.26, \textcircled{1}, 20 μM), GST-ST (SEQ ID No. 28, \textcircled{2}, 20 μM), and equal molar amounts of pre-mixed pSC (SEQ ID No. 26) and GST-ST (SEQ ID No. 28, 20 μM) that had been exposed to light (λ=365 nm, 20 mW cm⁻², 20 min: \textcircled{1}) or left in the dark (\textcircled{2}+\textcircled{3}), were mixed with KH₂PO₄ buffer (100 mM, pH 6.5), CDNB (1 mM), and reduced glutathione (1 mM). FIG. 10C: GST activity was calculated as the slope of sample absorbance (λ=340 nm) over 5 minutes and normalized to that of GST-ST (SEQ ID No. 28). No significant change to GST enzymatic activity were determined following LASL.

FIG. 11: LASL proceeds specifically in mammalian cell lysate. Reactivity of pSC (SEQ ID No. 26, \textcircled{1}) and GST-ST (SEQ ID No. 28, \textcircled{2}) (10 μM each, 37° C., 0.5 h) with and without light treatment (λ=365 nm, 20 mW cm⁻², 45 min) in HEK-293T mammalian cell lysate analyzed by SDS-PAGE. The LASL product is marked with \textcircled{3}.

FIG. 12A-12D: Photocontrol over intracellular protein localization via LASL with a membrane-bound pSC (SEQ ID No. 26). Optogenetic specification of protein membrane tethering in mammalian cells using a membrane-bound pSC (SEQ ID No. 26). FIG. 12A: Schematic of gene cassette used to prime cells for LASL-mediated plasma membrane labeling, where a CAAX-anchored pSC (SEQ ID No. 26) is covalently appended with cytosolic EGFP-ST upon light exposure. FIG. 12B: Representative fluorescent micrographs of transfected HEK-293T cells following varied light exposures (λ=365 nm, 20 mW cm², 0-20 min) and imaged 6 h after exposure. FIG. 12C: Membrane labeling with EGFP-ST scales in a statistically significant manner with light exposure, visualized as violin scatter plots. FIG. 12D: Intracellular EGFP-ST distribution transitions through LASL from uniformly cytosolic to more membrane localized with increased light treatment durations. Asterisks denote conditions with statistically significant differences in signal (p<0.05, unpaired t-tests). Scale bars, 20 μm.

FIG. 13. SDS-PAGE analysis of purified UnaG (SEQ ID No. 41) fragments for SpyLigation. Expression and purification of UnaG (SEQ ID No. 41) variants (SC-nUnaG (SEQ ID No. 35). SC-cUnaG (SEQ ID No. 37), nUnaG-SC (SEQ ID No. 36), cUnaG-SC (SEQ ID No. 38), nUnaG-ST (SEQ ID No. 31), cUnaG-ST) monitored through SDS-PAGE analysis with proteins visualized by Coomassie staining. Protein-containing lanes correspond to:
\textcircled{1} Pre-induction
\textcircled{2} Full cell lysate
\textcircled{3} Soluble fraction
\textcircled{4} Insoluble fraction
\textcircled{5} Column flow-through
\textcircled{6} Final wash
\textcircled{7} Elution/concentrated product

FIG. 14: SDS-PAGE analysis of purified wild-type UnaG (SEQ ID No. 41). Expression and purification of wild-type UnaG (SEQ ID No. 41) monitored through SDS-PAGE analysis with proteins visualized by Coomassie staining. Protein-containing lanes correspond to:
\textcircled{1} Pre-induction
\textcircled{2} Post-induction
\textcircled{3} Column flow-through
\textcircled{4} Soluble fraction
\textcircled{5} Insoluble fraction
\textcircled{6} First wash
\textcircled{7} Final wash
\textcircled{8} Elution/concentrated product

FIG. 15: SpyLigated UnaG (SEQ ID No. 41) fluorescence is reduced relative to wild-type UnaG (SEQ ID No. 41). To compare activity between the SpyLigated split and wild-type UnaG (SEQ ID No. 41), a solution of purified UnaG (SEQ ID No. 41, 10 UM, Tris Buffer) and equal molar amounts of nUnaG-ST (SEQ ID No. 31) and SC-cUnaG (SEQ ID No. 37, 10 μM. Tris Buffer) were independently mixed with the substrate bilirubin (20 μM final concentration). Fluorescence was measured 24 h after mixing (37° C.) in 96-well, black opaque plates with a fluorescent plate reader (λ_{excitation}=495 nm, λₑₘᵢₛₛᵢₒₙ=528 nm).

FIG. 16A-16B: Concentration-dependent split-UnaG (SEQ ID No. 41) fluorescence recovery with LASL. FIG. 16A: Split pSC-cUnaG (SEQ ID No. 39) and nUnaG-ST (SEQ ID No. 31) is exposed to light to trigger the reaction. FIG. 16B: Fluorescence signal of pSC-cUnaG (SEQ ID No. 39) and nUnaG-ST (SEQ ID No. 31) at varying equimolar concentrations (0-10 μM for each component) with (black circles) and without (outline circles) light (=365 nm, 20 mW cm⁻², 20 min). Analysis was performed immediately (open square) and after 24 h after light exposure. Data correspond to the S.D. about the mean for n=3 experimental replicates, normalized to the experimental minimum/maximum.

FIG. 17A-17B: Assessing background fluorescence in the UnaG (SEQ ID No. 41) LASL system. Some background fluorescence was observed in UnaG (SEQ ID No. 41) LASL components (i.e., nUnaG-ST (SEQ ID No. 31) and pSC-cUnaG, SEQ ID No. 39) that were never exposed to light. This could be attributed to one of several potential mechanisms: FIG. 17A: (1) non-covalent association of pSC (SEQ ID No. 26) and ST could locally concentrate UnaG (SEQ ID No. 41) fragments to promote re-assembly, (2) nUnaG and cUnaG fragments could weakly associate non-covalently in a manner stabilized with bilirubin and independent of the fused pSC (SEQ ID No. 26)/ST (SEQ ID No. 7, SEQ ID No. 8), or (3) some combination thereof. To assess, we pre-reacted pSC-cUnaG (SEQ ID No. 39) and SC-cUnaG (SEQ ID No. 37) with a synthetic SpyTag peptide (SEQ ID No. 7. SEQ ID No. 8, ST-N₃) (Methods S15-16). Since ST uniquely ligates with SC, we anticipated that this strategy would yield two “dead” SpyCatcher species—pSC-cUnaG (SEQ ID No. 39) caged with oNB and SC-cUnaG (SEQ ID No. 37) quenched with the full ST peptide—that could no longer undergo covalent reaction with nUnaG-ST (SEQ ID No. 31). FIG. 17B: Upon incubation with nUnaG-ST (SEQ ID No. 31), fluorescence of the wild-type SC-cUnaG (SEQ ID No. 37) system was reduced significantly (p<0.0001, unpaired t-test) to levels matching those of the photocaged variant when preconjugated with ST-N₃ peptide, whereas no reduction in fluorescence was observed for the pSC-cUnaG (SEQ ID No. 39) sample. Since similar background fluorescence was observed in both the oNB-caged and ST-N₃-quenched SC systems, background fluorescence was attributed to non-covalent association of UnaG fragments partially stabilized with bilirubin.

FIG. 18: Multiphoton patterning of split-UnaG activation via LASL in gels. UnaG is spatiotemporally reassembled within hydrogel biomaterials functionalized with pSC-cUnaG (SEQ ID No. 39) and patterned with nUnaG-ST (SEQ ID No. 31) via LASL. Multiphoton laser-scanning lithography affords patterned protein activation with full 3D control. Images represent maximum intensity projections of a 3D husky pattern across the indicated planes (xy, xz, and yz). Scale bar, 50 μm.

FIG. 19: SDS-PAGE analysis of purified split-NanoLuc fragments for LASL. Expression and purification of split-NanoLuc fragments (LgBiT-ST (SEQ ID No. 43) and pSC-SmBiT (SEQ ID No. 42)) variants monitored through SDS-PAGE analysis with proteins visualized by Coomassie staining. Protein-containing lanes correspond to.
\textcircled{1} Pre-induction
\textcircled{2} Full cell lysate
\textcircled{3} Soluble fraction
\textcircled{4} Insoluble fraction
\textcircled{5} Column flow-through
\textcircled{6} Final wash
\textcircled{7} Elution/concentrated product

FIG. 20A-20B: Concentration-dependent split-NanoLuc luminescence recovery with LASL. FIG. 20A: Split pSC-SmBiT (SEQ ID No. 42) and LgBiT-ST (SEQ ID No. 43) is exposed to light triggering the reaction. FIG. 20B: Luminescence signal of pSC-SmBiT (SEQ ID No. 42) and LgBiT-ST (SEQ ID No. 43) at varying equimolar concentrations (0-1 μM of each component) with (black circle) and without (outline circle) light treatment (λ=365 nm, 20 mW cm⁻², 20 min). Analysis was performed 24 h after light exposure. Error bars correspond to the S.D. about the mean for n=3 experimental replicates, with data normalized to the experimental minimum/maximum.

FIG. 21A-21B: Quantification of intracellular UnaG (SEQ ID No. 41)/mCh signal in light-(un)exposed cells. FIG. 21A: UnaG (SEQ ID No. 41) and mCh fluorescent values of individual cells were determined through automated image analysis and quantified over time (0-72 h after light exposures). Statistically significant increases for UnaG (SEQ ID No. 41)/mCh were observed for light-exposed cells within 30 min of exposure with peak levels detected by 3 h, but not for unexposed cells. FIG. 21B: Maximal UnaG (SEQ ID No. 41)/mCh fluorescence ratios (˜5-fold higher than unexposed samples) persisted for at least 72 h in a manner consistent with covalent UnaG (SEQ ID No. 41) assembly. mCh signal was found to decrease slightly over time, attributed to expected protein degradative clearance and/or dilution accompanying cell division. Asterisks denote conditions with statistically significant differences in signal (p<0.0001, unpaired t-tests).

FIG. 22A-22H: LASL-mediated UnaG (SEQ ID No. 41) activation does not significantly affect mCh signal. FIG. 22A: Mask-based photolithography spatiotemporally directs UnaG (SEQ ID No. 41) reassembly within HEK-293T cell culture. FIG. 22B-FIG. 22D: Fluorescent images of culture dish with inlays of exposure boundary magnified. FIG. 22B the darker gray representing the red luminescence from mCh. FIG. 22C the medium gray representing the green luminescence from UnaG (SEQ ID No. 41). FIG. 22D luminescence from both mCh and UnaG (SEQ ID No. 41). FIG. 22E: Individual cell UnaG (SEQ ID No. 41)/mCh and mCh signal quantified radially outwards from the mask's center, normalized to average unexposed UnaG (SEQ ID No. 41)/mCh ratio and FIG. 22F: mCh. Dashed line indicates exposure edge. FIG. 22G-FIG. 22H: Violin scatter plots of normalized UnaG (SEQ ID No. 41)/mCh ratios and mCh in light-(un)exposed regions. FIG. 22G: Significant differences between UnaG (SEQ ID No. 41)/mCh ratios were observed between regions, FIG. 22H: but not for mCh fluorescence. Light treatments, 20 mW cm⁻² , λ=365 nm, 20 min. Asterisks denote conditions with statistically significant differences in signal (p<0.0001, unpaired t-tests). Scale bars, 1 mm.

### DETAILED DESCRIPTION

Biology is comprised of a series of well-orchestrated chemical reactions that are precisely controlled in time and three-dimensional space (i.e., 4D). Proteins act as the conductors of these reactions, providing essential and unmatched functional regulation of many bioprocesses across all scales of life. As proteins offer structural integrity, regulate gene expression, and serve as the central language of cellular communication, there is little question as to why global research efforts continue to seek to improve existing and develop new techniques to regulate protein function within living systems. While systematic edits to the genome can enable long-lasting over- and underexpression of proteins in vitro and in vivo, these efforts require long times ranging from many hours to weeks; critical need remains for systems that permit real-time modulation of protein function in a user-defined manner.

A growing and powerful trend towards the induction of bioactivity in living cells involves triggered protein re-assembly from non-functional fragment pairs. In this strategy, proteins are genetically “split” into biologically inactive fragments whose negligible affinity prevents spontaneous re-assembly but can be complexed into a functional species under specific conditions. Currently, controlled reassembly of split proteins is commonly achieved through genetic fusion of split fragments with inducible dimerizers, whereby exogenously triggered protein dimerization brings fragments into proximity to restore function of the split parent species. Most frequently, these methods exploit small-molecule chemical ligands for inducible dimerization (e.g., rapamycin-induced heterodimerization of FKBP/FRB, coumermycin homodimerization of GyrB). Though specification over when small-molecule inducers are added to culture affords temporal control over split protein activity, such chemical activation cannot be readily regulated in 2D or 3D space. To address this limitation, several optogenetic strategies have been developed that employ light-responsive proteins (e.g., PhyB-PIF, Cry2/CIB1, Magnets) that bind under visible-light irradiation. While optogenetic approaches have already enabled a seemingly limitless collection of exciting new studies, they are not without limitations: (1) optogenetic proteins are often quite large (>500 amino acids per partner), limiting expression efficiency and posing potential concern over reaction sterics; (2) previously developed systems are universally sensitive to visible light (λ=450-650 nm), making them practically difficult to work with under standard laboratory lighting and dramatically limiting their combined utility with common green/orange/red-type fluorophores; (3) reactions are non-covalent and quickly reverse under dark conditions (often with half-lives of seconds to minutes), with continuous illumination for sustained protein activation that presents challenges with long-term culture or migrating systems, (4) overall extent of reaction is near-impossible to control, rendering intermediate activation states inaccessible, and (5) light-responsive proteins typically show minimal-to-no responsiveness to multiphoton activation, rendering full 3D spatial modulation largely out of reach.

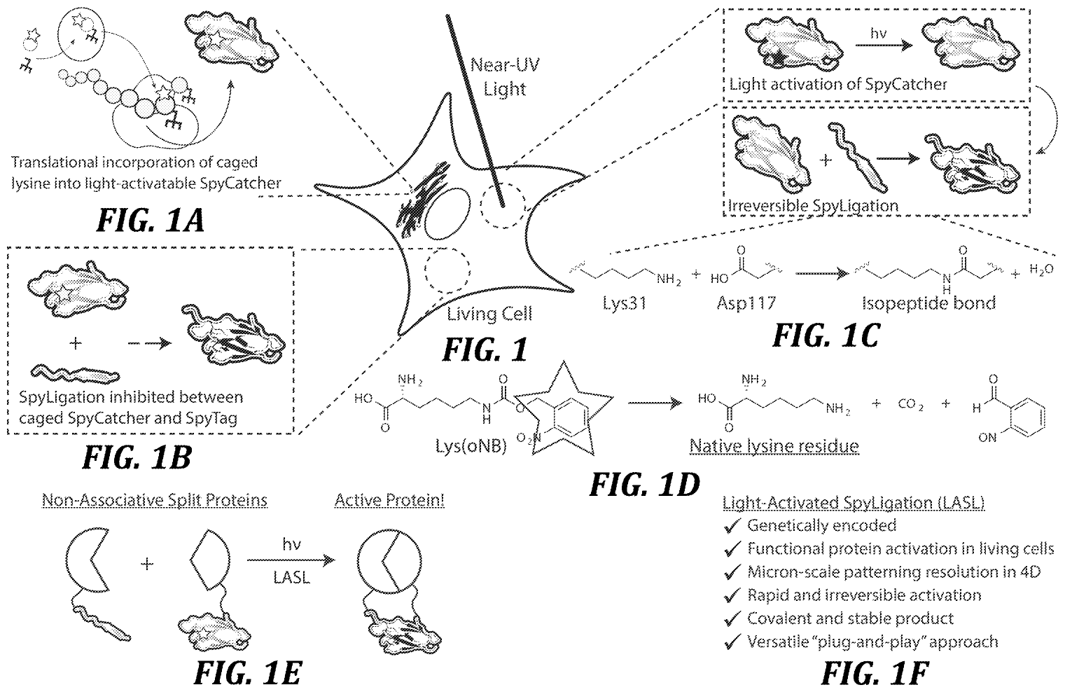

Many of the limitations to existing optogenetic approaches could be addressed through chemical advances, we sought to establish a genetically encoded protein-protein ligation reaction involving small partners that could be optically regulated in a rapid, irreversible, dose-dependent, and highly specific manner. Towards this goal, we were inspired by the versatility of “SpyLigation”, in which genetically encoded SpyCatcher (SC. 113 amino acids. 12.1 kDa) and SpyTag (SEQ ID No. 7, SEQ ID No. 8, ST, 13 amino acids, 1.5 kDa) protein pairs undergo spontaneous covalent coupling with high yield in living cells (mammalian, bacterial, and plant) that is maintained amidst diverse conditions of pH, temperature, and buffer. Though recent efforts to shield ST with the light-responsive AsLOV2 protein have afforded some photocontrol over SpyLigation, significant “dark reaction” precludes the strategy's functional utilization in many contexts including those within living cells and 3D materials. As SpyLigation involves isopeptide bond formation between a critical lysine on SC (Lys31) and an essential aspartic acid on ST, complete optical control over SpyLigation could be obtained through molecular “photocaging” of either residue, chemically blocking the reactive side chains with a photoremovable moiety. Since genetic code expansion can be used to site-specifically install non-canonical amino acids at user-defined locations within proteins during translation, and requisite tRNA/tRNA synthetase pairs have been previously evolved for efficient incorporation of photocaged lysine residues at the rarely utilized amber stop codon within bacterial and mammalian culture, a photoactivatable SC (pSC, SEQ ID No. 26) could be created through amber suppression whose catalytic Lys31 residue was substituted with an ortho-nitrobenzyl (oNB)-caged analog [Nε-(o-nitrobenzyloxycarbonyl)-L-lysine, Lys(oNB)]. We expected that pSC's (SEQ ID No. 26) photocage would prevent isopeptide bond formation with ST but could be rapidly removed in response to cytocompatible near-ultraviolet light (near-UV, λ=365 nm) or near-infrared (NIR) multiphoton stimulation to yield the functional SC in a dose-dependent manner and with 4D control. When used to photoligate split protein pairs, we anticipated that this “light-activated SpyLigation” (LASL) could be exploited to irreversibly assemble proteins and restore function in solution, in biomaterials, and intracellularly with spatiotemporal control, expanding current capabilities and opening new doors to probe and direct cellular fate (FIG. 1).

### Definitions

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. As an example, the term “optionally substituted with 1, 2, 3, 4, or 5” is intended to individually disclose optionally substituted with 1, 2, 3, 4, or 5; 1, 2, 3, or 4; 1, 2, or 3; 1 or 2; or 1 substituents.

It is further intended that the compounds of the disclosure are stable. As used herein “stable” refers to a compound that is sufficiently robust to survive isolation to a useful degree of purity from a reaction mixture.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As used herein, the term “activating agent” refers to a chemical compound which is capable of activating, for example, one or more carboxyl groups within carboxylic acids or carboxylic acid derivatives for nucleophilic reactions, wherein preferably said carboxyl groups include —C(O)X groups, wherein X═OH, halo (e.g., I, Br, Cl), OR (e.g., an anhydride), NH₂ or NH—R. In some embodiments, the carbonyl group is within a chloroformate. An “activated” functional group is a functional group that has been reacted with an activating agent. The activated functional group has a lower barrier to reacting with a nucleophile compared to an unactivated functional group.

As used herein, the term “natural polymer.” “naturally derived polymer,” or “naturally sourced polymer” refers to polymers found in nature

As used herein, the term “caging group” or “caging” refers to a moiety that can be employed to reversibly block, inhibit, or interfere with the activity (e.g., the chemical reactivity) of a molecule (e.g., a polypeptide, a nucleic acid, a small molecule, a drug, and the like); and its respective process. Typically, one or more caging groups are associated (covalently or noncovalently) with the molecule but do not necessarily surround the molecule in a physical cage. Caging groups can be removed from a molecule, or their interference with the molecule's activity can be otherwise reversed or reduced, by exposure to an appropriate type of uncaging energy and/or exposure to an uncaging chemical, enzyme, or the like. Examples of caging groups that can be used in the heterobifunctional linker are described, for example, in Dynamic Studies in Biology: Phototriggers, Photoswitches, and Caged Biomolecules Edited by Maurice Goeldner (Universite L. Pasteur Strasbourg, France) and Richard Givens (University of Kansas. USA). Wiley-VCH GmbH & Co. KgaA: Weinheim. 2005, incorporated herein by reference in its entirety.

As used herein, the term“photocaging” refers to a caging group that is removed by exposing the caging group to light of a predetermined wavelength.

As used herein, the term “linker” refers to atoms or molecules that link or bond two entities (e.g., hydrogel, hydrogel label, solid supports, oligonucleotides, or other molecules), but that is not a part of either of the individual linked entities.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl. The term “substituted” in reference to alkyl, alkylene, aryl, arylalkyl, alkoxy, heterocyclyl, heteroaryl, etc., for example, “substituted alkyl”, “substituted alkylene”, “substituted aryl”, “substituted arylalkyl”, “substituted heterocyclyl”, and “substituted heteroaryl” means alkyl, alkylene, aryl, arylalkyl, heterocyclyl, heteroaryl respectively, in which one or more hydrogen atoms are each independently replaced with a non-hydrogen substituent. Typical substituents include, but are not limited to, alkyl, alkenyl, alkynyl, —Z, —R′, —O—, ═O⁻, —OR′, —SR′, —S′, —NR′₂, —N⁺R′₃, ═NR′, —CZ₃, —CN, —OCN, —SCN, —N═C=O, —NCS, —NO, —NO₂, ═N₂, —N₃, —NHC(═O)R′, —OC(═O)R′, —NHC(═O)NR′₂, —S(═O)₂—, —S(═O)₂OH, —S(═O)₂R′, —OS(═O)₂OR′, —S(═O)₂NR′₂, —S(═O)R′, —OP(═O)(OR′)₂, —P(═O)(OR′)₂, —P(═O)(O⁻), —P(═O)(OH)₂, —P(O)(OR′)(O), —C(═O)R′, —C(═O)Z, —C(S)R′, —C(O)OR′, —C(O)O—, —C(S)OR′, —C(O)SR′, —C(S)SR′, —C(O)NR′₂, —C(S)NR′₂. —C(═NR″)NR′₂, where each Z is independently a halogen: F, Cl, Br, or I. and each R′ is independently H, alkyl, aryl, arylalkyl, a heterocycle, or a protecting group. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

“Optionally substituted” groups can refer to, for example, functional groups that may be substituted or unsubstituted by additional functional groups. For example, when a group is unsubstituted, it can be referred to as the group name, for example alkyl or aryl. When a group is substituted with additional functional groups, it may more generically be referred to as substituted alkyl or substituted aryl.

Where a numerical range is disclosed herein, then such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of I and the maximum value of 10. Exemplary subranges of the range “to 10” include, but are not limited to, e.g., 1 to 6.1, 3.5 to 7.8, and 5.5 to 10.

It is intended that divalent groups, such as linking groups (e.g., alkylene, arylene, etc.) between a first and a second moieties, can be oriented in both forward and the reverse direction with respect to the first and second moieties, unless specifically described.

Terms used herein may be preceded and/or followed by a single dash, “−”, or a double dash, “=”, to indicate the bond order of the bond between the named substituent and its parent moiety, a single dash indicates a single bond and a double dash indicates a double bond. In the absence of a single or double dash it is understood that a single bond is formed between the substituent and its parent moiety: further, substituents are intended to be read “left to right” unless a dash indicates otherwise. For example, C₁-C₆ alkoxycarbonyloxy and —OC(O)C₁-C₆ alkyl indicate the same functionality; similarly arylalkyl and -alkylaryl indicate the same functionality.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein. “alkynylene” refers to a linking alkynyl group.

As used herein, the term “amino” refers generally to a nitrogen radical which can be considered a derivative of ammonia, having the formula —N(Y)₂, where each “Y” is independently H, substituted or unsubstituted alkyl, substituted or unsubstituted carbocyclyl, substituted or unsubstituted heterocyclyl, etc. The hybridization of the nitrogen is approximately sp³. Nonlimiting types of amino include —NH₂, —N(alkyl)₂, —NH(alkyl), —N(carbocyclyl)₂, —NH(carbocyclyl), —N(heterocyclyl)₂, —NH(heterocyclyl), —N(aryl)₂, —NH(aryl), —N(alkyl)(aryl), —N(alkyl)(heterocyclyl), —N(carbocyclyl)(heterocyclyl), —N(aryl)(heteroaryl), —N(alkyl)(heteroaryl), etc. The term “alkylamino” refers to an amino group substituted with one alkyl group. The term “dialkylamino” refers to an amino group substituted with two alkyl groups. Nonlimiting examples of amino groups include —NH₂, —NH(CH₃), —N(CH₃)₂, —NH(CH₂CH₃), —N(CH₂CH₃)₂, —NH(phenyl), —N(phenyl)₂. —NH(benzyl), —N(benzyl)₂, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O-alkyl), —N(alkylene-C(O)—OH)₂, —N(alkylene-C(O)—O— alkyl)₂, etc.

As used herein, the term “aryl” refers to monocyclic or polycyclic (e.g., having 2, 3 or 4 fused rings) aromatic hydrocarbon radical derived by the removal of one hydrogen atom from a single carbon atom of a parent aromatic ring system. For example, an aryl group can include example, phenyl, naphthyl, anthracenyl, phenanthrenyl, indanyl, indenyl, and the like. In some embodiments, aryl groups have from 6 to about 20 carbon atoms, 6 to 14 carbon atoms, or 6 to 10 carbon atoms.

As used herein, the term “arylene” refers to a linking aryl group.

As used herein, the term “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, the term “cycloalkylene” refers to a linking cycloalkyl group.

As used herein, the term “heteroalkyl” refers to an alkyl group having at least one heteroatom such as sulfur, oxygen, or nitrogen. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

As used herein, the term “heteroalkylene” refers to a linking heteroalkyl group.

As used herein, the term “heteroaryl” refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyO, pyrryl, oxazolyl, benzofuryl, benzothienyl, benzthiazolyl, isoxazohyl, pyrazolyl, triazolyl, tetrazolyl, indazolyl, 1,2,4-thiadiazolyl, isothiazolyl, benzothienyl, purinyl, carbazolyl, benzimidazolyl, indolinyl, acridinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, the term “heteroarylene” refers to a linking heteroaryl group.

As used herein, the term “alkoxy” refers to an —O-alkyl group. Example alkoxy groups include methoxy, ethoxy, propoxy (e.g., n-propoxy and isopropoxy), t-butoxy, and the like.

As used herein, the term “cycloalkoxy” refers to an —O-cycloalkyl group.

As used herein, the term “heterocycloalkoxy” refers to an —O-heterocycloalkyl group.

As used herein, the term “aryloxy” refers to an —O-aryl group. Example aryloxy groups include phenyl-O—, substituted phenyl-O—, and the like.

As used herein, the term “heteroaryloxy” refers to an —O-heteroaryl group.

As used herein, the term “arylalkyl” refers to alkyl substituted by aryl and “cycloalkylalkyl” refers to alkyl substituted by cycloalkyl. An example arylalkyl group is benzyl.

As used herein, the term “heteroarylalkyl” refers to alkyl substituted by heteroaryl and “heterocycloalkylalkyl” refers to alkyl substituted by heterocycloalkyl.

As used herein, the term “halo” or “halogen” includes fluoro, chloro, bromo, and iodo.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, an “electron donating substituent” refers to a substituent that adds electron density to an adjacent pi (π)-system, making the n-system more nucleophilic. In some embodiments, an electron donating substituent has lone pair electrons on the atom adjacent to π-system. In some embodiments, electron donating substituents have n-electrons, which can donate electron density to the adjacent pi-system via hyperconjugation. Examples of electron donating substituents include O—, NR₂, NH₂, OH, OR, NHC(O)R, OC(O)R, aryl, and vinyl substituents.

As used herein, the term “unsaturated bond” refers to a carbon-carbon double bond or a carbon-carbon triple bond.

As used herein, the term “protecting group” refers to a moiety of a compound that masks or alters the properties of a functional group or the properties of the compound as a whole. The chemical substructure of a protecting group varies widely. One function of a protecting group is to serve as an intermediate in the synthesis of the parental drug substance. Chemical protecting groups and strategies for protection/deprotection are described, for example, in “Protective Groups in Organic Chemistry,” Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. “Hydroxy protecting groups” refers to those protecting groups useful for protecting hydroxy groups (—OH).

As used herein, “forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

As used herein, a “leaving group” refers to groups that maintain the bonding electron pair during heterolytic bond cleavage. For example, a leaving group is readily displaced during a nucleophilic displacement reaction. Suitable leaving groups include, but are not limited to, chloride, bromide, mesylate, tosylate, triflate, 4-nitrobenzenesulfonate, 4-chlorobenzenesulfonate, 4-nitrophenoxy, pentafluorophenoxy, etc. One of skill in the art will recognize other leaving groups useful in the present disclosure.

As used herein, a “deprotection agent” refers to any agent capable of removing a protecting group. The deprotection agent will depend on the type of protecting group used. Representative deprotection agents are known in the art and can be found in Protective Groups in Organic Chemistry, Peter G. M. Wuts and Theodora W. Greene, 4^{th} Ed., 2006.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O—corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue, and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “peptide” refers to natural biological or artificially manufactured short chains of amino acid monomers linked by peptide (amide) bonds. As used herein, a peptide has at least 2 amino acid repeating units.

As used herein, the term “oligomer” refers to a macromolecule having 10 or less repeating units.

As used herein, the term “polymer” refers to a macromolecule having more than 10 repeating units.

As used herein, the term “polysaccharide” refers to a carbohydrate that can be decomposed by hydrolysis into two or more molecules of monosaccharides.

As used herein, the term “hydrogel” refers to a water-swollen, and cross-linked polymeric network produced by the reaction of one or more monomers. The polymeric material exhibits the ability to swell and retain a significant fraction of water within its structure, but does not dissolve in water.

As used herein, the term “protein” refers to any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “tissue” refers to an aggregate of similar cells and cell products forming a definite kind of structural material with a specific function, in a multicellular organism.

As used herein, the term “organs” refers to a group of tissues in a living organism that have been adapted to perform a specific function.

As used herein, the term “therapeutic agent” refers to a substance capable of producing a curative effect in a disease state.

As used herein, the term “small molecule” refers to a low molecular weight (<2000 daltons) organic compound that may help regulate a biological process, with a size on the order of 1 nm. Most drugs are small molecules.

As used herein, the term “biomaterial” refers to a natural or synthetic material (such as a metal or polymer) that is suitable for introduction into living tissue, for example, as part of a medical device (such as an artificial joint).

As used herein, the term “ceramic” refers to an inorganic, non-metallic, solid material comprising metal, non-metal or metalloid atoms primarily held in ionic and covalent bonds.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “chelating agent” refers to a ligand that forms two or more separate coordinate bonds to a single central metal ion.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease: for example, ameliorating a disease, condition or disorder in an individual who is experiencing or displaying the pathology or symptomatology of the disease, condition or disorder (i.e., reversing the pathology and/or symptomatology) such as decreasing the severity of disease.

The compounds described herein can be asymmetric (e.g., having one or more stereocenters). All stereoisomers, such as enantiomers and diastereomers, are intended unless otherwise indicated.

Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and can be isolated as a mixture of isomers or as separated isomeric forms.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole. 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. For example, isotopes of hydrogen include tritium and deuterium.

In some embodiments, the compounds of the disclosure, and salts thereof, are substantially isolated. By “substantially isolated” is meant that the compound is at least partially or substantially separated from the environment in which it was formed or detected. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

Unless defined otherwise, any feature within any aspect or embodiment of the disclosure may be combined with any feature within any other aspect or embodiment of the disclosure, and such combination are encompassed in the present disclosure. This also applies, but not exclusively, to endpoints of ranges disclosed herein. For instance, if a given substance is disclosed as existing in a composition in a concentration range of X-Y % or A-B %, the present disclosure is to be understood as explicitly disclosing not only the ranges X-Y % and A-B %, but also the ranges X-B %, A-Y % and, in as far as numerically possible, Y-A % and B-X %. Each of these ranges, and range combinations, are contemplated, and are to be understood as being directly and unambiguously disclosed in the present application.

Unless stated otherwise, the designation of a range in the present application using a hyphen (“−”) separating two bracketing values X and Y, or two bracketing ratios, is to be understood as meaning and disclosing the specified range in which both endpoint values X and Y are included. The same applies to a range expressed as “from X to Y”. Accordingly, the expressions of ranges as “X-Y”. “of X to Y”, “from X to Y”, “of X-Y” and “from X-Y” are to be understood equivalently as meaning and disclosing a range encompassing the end value X, all values (including decimals) between X and Y, as well as the end value Y.

As used herein the term “about” when referring to a particular value, e.g., an endpoint or endpoints of a range, encompasses and discloses, in addition to the specifically recited value itself, a certain variation around that specifically recited value. Such a variation may for example arise from normal measurement variability, e.g., in the weighing or apportioning of various substances by methods known to the skilled person. The term “about” shall be understood as encompassing and disclosing a range of variability above and below an indicated specific value, said percentage values being relative to the specific recited value itself, as follows: The term “about” may encompass and disclose variability of 5.0%. The term “about” may encompass and disclose variability of 4.5%. The term “about” may encompass and disclose variability of f 4.0%. The term “about” may encompass and disclose variability of +3.5%. The term “about” may encompass and disclose variability of t 3.0%. The term “about” may encompass and disclose variability of 2.5%. The term “about” may encompass and disclose variability of 2.0%. The term “about” may encompass and disclose variability of f 1.5%. The term “about” may encompass and disclose variability of +1.0%. The term “about” may encompass and disclose variability of ±0.5%. The term “about”, in reference to the particular recited value, may encompass and disclose that exact particular value itself, irrespective of any explicit mention that this exact particular value is included: even in the absence of an explicit indication that the term “about” includes the particular exact recited value, this exact particular value is still included in the range of variation created by the term “about”, and is therefore disclosed in the present application. Unless stated otherwise, where the term “about” is recited before the first endpoint of a numerical range, but not before the second endpoint of that range, this term, and the variability it implies in scope and disclosure, refers to both the first endpoint of the range and the second endpoint of the range. For instance, a recited range of “about X to Y” should be read as “about X to about Y”. The same applies for a recited range of ratios. For instance, a recited range of weight ratios of “about X:Y−A:B” should be read as a weight ratio of “(about X):(about Y)−(about A) [symbol] about B)”.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

In one aspect, an exogenously triggerable self-assembling protein construct is provided, comprising:
a caged reactive first protein fragment comprising a first stimulus-responsive cleavable moiety capable of cleaving from the caged reactive first protein fragment, upon application of a predetermined first stimulus, to provide a reactive first protein fragment;
a first split protein linked with the caged reactive first protein fragment;
a complementary reactive second protein fragment capable of reacting with the first reactive protein fragment, and
a second split protein linked with the complementary reactive second protein fragment,
wherein the first reactive protein fragment is adapted to react covalently with the complementary reactive second protein fragment to provide a self-assembled ligated protein or a portion thereof; and
wherein the first split protein is adapted to associate with the second split protein and to form an active protein in accordance with the reaction of the first reactive protein fragment and the complementary reactive second protein fragment providing the self-assembled protein or the portion thereof. LASL (Spatiotemporal Functional Assembly of Split Protein Pairs through a Light-Activated SpyLigation) is an exemplary embodiment of self-assembling protein construct as disclosed herein. LASL is a self-assembling protein construct where photons activate the protein pairs to assemble. With LASL there is a caged reactive protein fragment that responds to the incidence of photons. The photons remove the cage allowing the protein constructs to assemble.

In some nonlimiting embodiments the self-assembling protein construct there is the first split protein and the second split protein respectively comprise fragments of a first fluorescent protein, and wherein the active protein comprises the first fluorescent protein.

In some nonlimiting embodiments the self-assembling protein construct here is the first split protein comprises a second fluorescent protein and the second split protein comprises a third fluorescent protein, and wherein the active protein comprises the self-assembled protein, the second fluorescent protein, and the third fluorescent protein.

In some embodiments the self-assembling protein construct comprises, but not limited to, the first fluorescent protein, the second fluorescent protein, or the third fluorescent protein respectively comprises but not limited to EGFP, UnaG (SEQ ID No. 41), mCherry. or mRuby and any combination thereof.

In some embodiments the self-assembling protein construct comprises the first split protein and the second split protein respectively comprise inactive fragments of a luminescent protein, and wherein the active protein comprises the luminescent protein.

In some embodiments the self-assembling protein construct is luciferase.

The self-assembling protein construct in some nonlimiting embodiments comprises the first split protein and the second split protein respectively comprise inactive fragments of an enzyme, and wherein the active protein comprises the enzyme.

The self-assembling protein construct comprises the enzyme is a DNA recombinase in some nonlimiting embodiments.

The self-assembling protein construct is made of in nonlimiting examples of the caged reactive first protein fragment, the complementary reactive second protein fragment, the first split protein, or the second split protein is coupled with a biomaterial or a biocompatible material.

The self-assembling protein construct is made of biomaterial or the biocompatible material comprises a lipid bilayer, a hydrogel, or a cell membrane in some nonlimiting embodiments.

The self-assembling protein construct comprises of but is not limited to a first stimulus-responsive cleavable moiety is selected from a group consisting of a photo-cleavable moiety, an enzyme-cleavable moiety, a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, a hydrolyzable moiety, a transition metal-triggered cleavage reaction-cleavable moiety, a cycloaddition-mediated cleavage reaction-cleavable moiety, and any combination thereof.

The self-assembling protein construct comprises but is not limited to the first stimulus-responsive cleavable moiety comprises an matrix metalloproteinase (MMP}-cleavable sequence; a cathepsin-cleavable sequence; an elastase-cleavable sequence; a disulfide moiety; a thioketal moiety; a nitrobenzyl moiety; a coumarin moiety; a hydrazone moiety; an oxime moiety; an acetal moiety; a silyl ether moiety; a transcyclooctene moiety; an ester moiety, and any combination thereof.

The self-assembling protein construct embodies a stimulus-responsive cleavable moiety comprises of but is not limited to Nε-(o-nitrobenzyloxycarbonyl), 2-nitrobenzyl, 3-nitrobenzyl, 4-nitrobenzyl, 2,3-dinitrobenzyl, 2,4-dinitrobenzyl, 2,6-dinitrobenzyl, 2-nitro-4,5-dimethoxybenzyl, 6-nitrobenzo[d][1,3]dioxol-5-yl, benzyl, naphthyl, anthryl, phenanthryl, pyrene, perylene, coumarin, caffeic acid chlorambucil or any one of the structures below

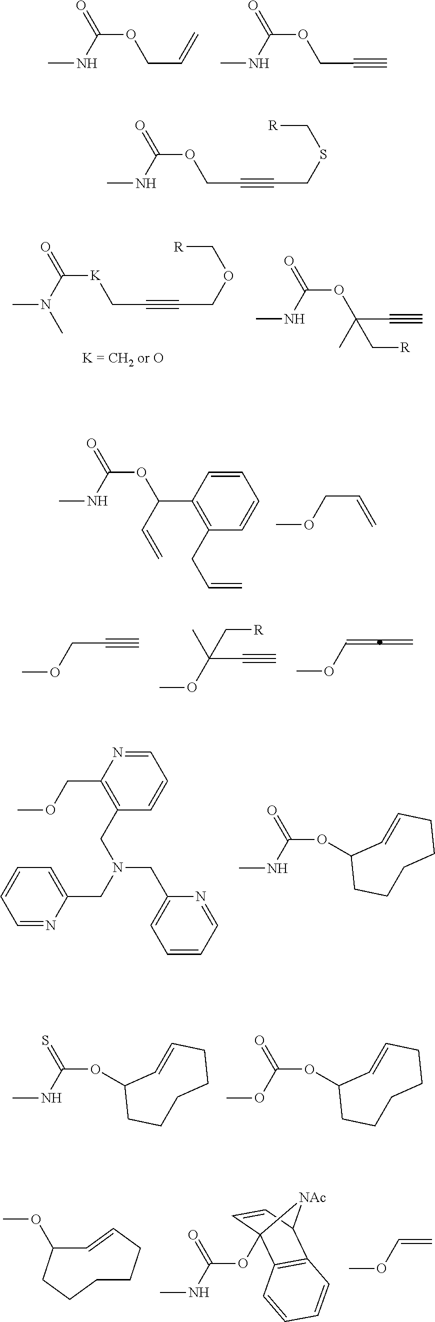

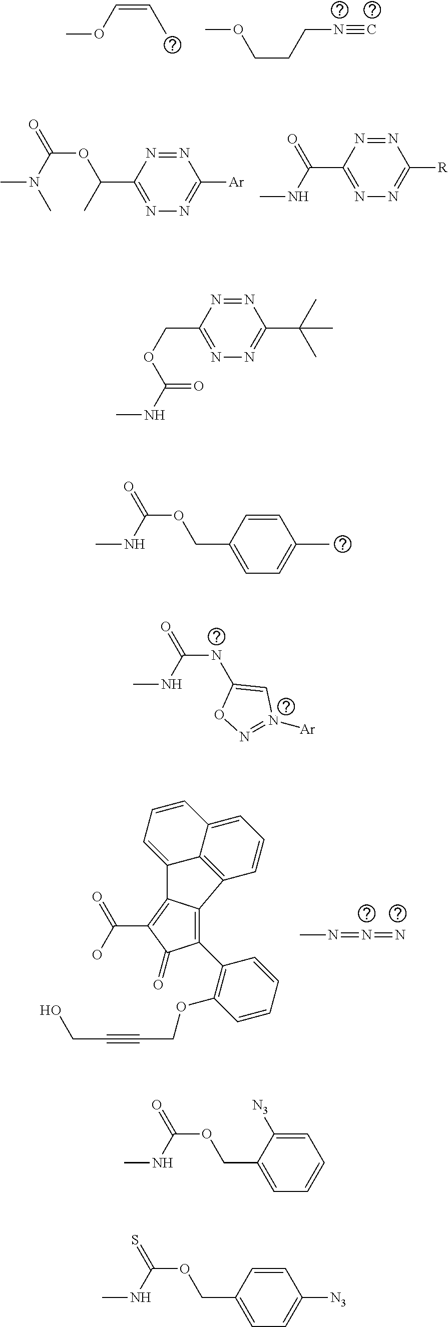

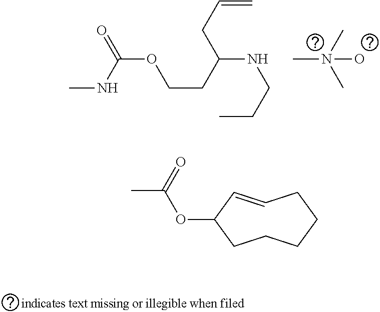

and any combination thereof.

Self-assembling protein constructs in nonlimiting example where the reactive first protein fragment comprises a first reactive moiety; and the complementary reactive second protein fragment comprises a second reactive moiety; and the first and second reactive moieties are capable of reacting to form a covalent bond.

In some nonlimiting embodiments the self-assembling protein construct is made of a reactive first protein fragment and the complementary reactive second protein fragment respectively comprise a ligating sequence selected from a SpyCatcher sequence, a SpyCatcher002 sequence, SpyCatcher003 sequence, SpyCatcher^{DDDDK} Sequence, a SpyCatcher ΔN1ΔC1 sequence, a DogCatcher sequence, a SpyStapler sequence, a SpyLigase sequence, a SnoopLigase sequence, a transglutaminase factor XIII, a sortase recognition sequence, a butelase recognition sequence, a OaAEP1b recognition sequence, a SpyTag (SEQ ID No. 7, SEQ ID No. 8) sequence, a SpyTag002 sequence, a SpyTag003 sequence, a DogTag sequence, a BDTag sequence, a KTag sequence, a SnoopTag sequence, a SdyTag sequence, a SdyCatcher sequence, or a NeissLock sequence; wherein the reactive first and second protein fragments are complementarily reactive to each other and any combination thereof.

In one embodiment comprises of, but not limited a self-assembling protein construct where:
a first split protein comprises a first portion of an UnaG (SEQ ID No. 41) fluorescent protein, the first portion comprising an N-terminus of the UnaG (SEQ ID No. 41) fluorescent protein;
the first split protein is bound to a C-terminus of the reactive first protein fragment;
a second split protein comprises a second portion of the UnaG (SEQ ID No. 41) fluorescent protein, the second portion comprising a C-terminus of the UnaG (SEQ ID No. 41) fluorescent protein; and
the second split protein is bound to an N-terminus of the complementary reactive second protein fragment.

The self-assembling protein construct where in one embodiment the caged reactive first protein fragment and the complementary reactive second protein fragment are non-functional.

The self-assembling protein construct where in one embodiment the self-assembled protein is a functional protein.

The self-assembling protein construct where there is a stimulus responsive cage and the predetermined stimulus is selected from: electromagnetic radiation, biocompatible electromagnetic radiation, an enzyme, a redox-active reagent (e.g., an electron donor, an electron acceptor), an acid, a base, a nucleophilic molecule, an electrophilic molecule, a chelating agent, a predetermined temperature, water, a transition metal, tetrazine, a cycloalkene, a cycloalkyne, a cyanoalkylsilane, a ketone, a phosphinyl compound, (BPIN)2 and any combination thereof.

In some nonlimiting embodiment the self-assembling protein construct can further comprise one or more additional caged reactive protein fragments and one or more complementary reactive second protein fragments.

In one embodiment a complementary reactive second protein fragment that is caged and comprises a second stimulus-responsive cleavable moiety capable of cleaving from the caged reactive second protein fragment upon application of a predetermined second stimulus to provide the complementary reactive second protein fragment.

In one embodiment the self-assembling protein construct has a first stimulus-responsive cleavable moiety and the second stimulus-responsive cleavable moiety where the first and second cleavable moieties are the same.

In one embodiment the self-assembling protein construct has the predetermined first and second stimuli as the same stimuli.

In one aspect, a method of controlling protein function in the described self-assembling protein construct system is provided and comprises: applying a predetermined stimulus to the self-assembling protein construct at a predetermined time and location, where the first reactive protein fragment and the complementary reactive second protein fragment self-assemble to provide the functional protein or a portion thereof and the active protein.

Self-assembling protein constructs can be made of protein fragments, caging moieties and self-assembling protein constructs. In some non-limiting embodiments these moieties can comprise of caging a first non-functional protein fragment to provide the caged reactive first protein fragment, linking the first non-functional protein fragment with the first split protein, and providing a complementary reactive second protein fragment where the complementary reactive second protein fragment is linked with the second split protein.

A nonlimiting method of employing LASL by caging a first non-functional protein fragment comprises recombinantly expressing the first non-functional protein fragment with a ligating sequence comprising a first stimulus-responsive cleavable moiety.

The method embodying caging a first non-functional protein fragment where the first non-functional protein fragment comprises recombinantly expressing the first non-functional protein fragment with a ligating sequence, followed by reacting the ligating sequence with a first stimulus-responsive cleavable moiety.

The method embodying caging a second non-functional protein fragment to provide the caged reactive second protein fragment.

In one embodiment self-assembling protein construct can cage a second non-functional protein where caging a second non-functional protein fragment comprises recombinantly expressing the second non-functional protein fragment with a ligating sequence comprising a second stimulus-responsive cleavable moiety.

In one embodiment self-assembling protein construct can cage a second non-functional protein. The caging a second non-functional protein fragment can comprise recombinantly expressing the second non-functional protein fragment with a ligating sequence, followed by reacting the ligating sequence with a second stimulus-responsive cleavable moiety.

In some embodiments self-assembling protein construct comprises of a complementary reactive second protein fragment comprises recombinantly expressing the second protein fragment with a complementary reactive ligating sequence.

Some nonlimiting ligating sequence species can be selected from a SpyCatcher sequence, a SpyCatcher002 sequence, SpyCatcher003 sequence, SpyCatcher^{DDDDK} sequence, a SpyCatcher ΔN1ΔC1 sequence, a DogCatcher sequence, a SpyStapler sequence, a SpyLigase sequence, a SnoopLigase sequence, a transglutaminase factor XIII, a sortase recognition sequence, a butelase recognition sequence, a OaAEPIb recognition sequence, a SpyTag (SEQ ID No. 7, SEQ ID No. 8) sequence, a SpyTag002 sequence, a SpyTag003 sequence, a DogTag sequence, a BDTag sequence, a KTag sequence, a SnoopTag sequence, a SdyTag sequence, a SdyCatcher sequence, a NeissLock sequence and any combination thereof.

In some nonlimiting embodiment the first stimulus-responsive cleavable moiety and the second stimulus-responsive cleavable moiety (when present) are independently selected from a photo-cleavable moiety, an enzyme-cleavable moiety, a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, a hydrolyzable moiety, a transition metal-triggered cleavage reaction-cleavable moiety, cycloaddition-mediated cleavage reaction-cleavable moiety and any combination thereof.

Self-assembling protein constructs make use of stimulus-responsive cleavable moieties and some nonlimiting examples of this utilization are first stimulus-responsive cleavable moiety and the second stimulus-responsive cleavable moiety (when present) each independently comprises an MMP-cleavable sequence; a cathepsin-cleavable sequence, an elastase-cleavable sequence, a disulfide moiety, a thioketal moiety, a nitrobenzyl moiety, a coumarin moiety, a hydrazone moiety, an oxime moiety, an acetal moiety, a silyl ether moiety, a transcyclooctene moiety, an ester moiety, and any combination thereof.

In one nonlimiting embodiment there is a method where the first stimulus-responsive cleavable moiety and the second stimulus-responsive cleavable moiety (when present) each independently comprises of but is not limited to Nε-(o-nitrobenzyloxycarbonyl), 2-nitrobenzyl, 3-nitrobenzyl, 4-nitrobenzyl, 2,3-dinitrobenzyl, 2,4-dinitrobenzyl, 2,6-dinitrobenzyl, 2-nitro-4,5-dimethoxybenzyl, 6-nitrobenzo[d][1,3]dioxol-5-yl, benzyl, naphthyl, anthryl, phenanthryl, pyrene, perylene, coumarin, caffeic acid chlorambucil or any one of the structures below

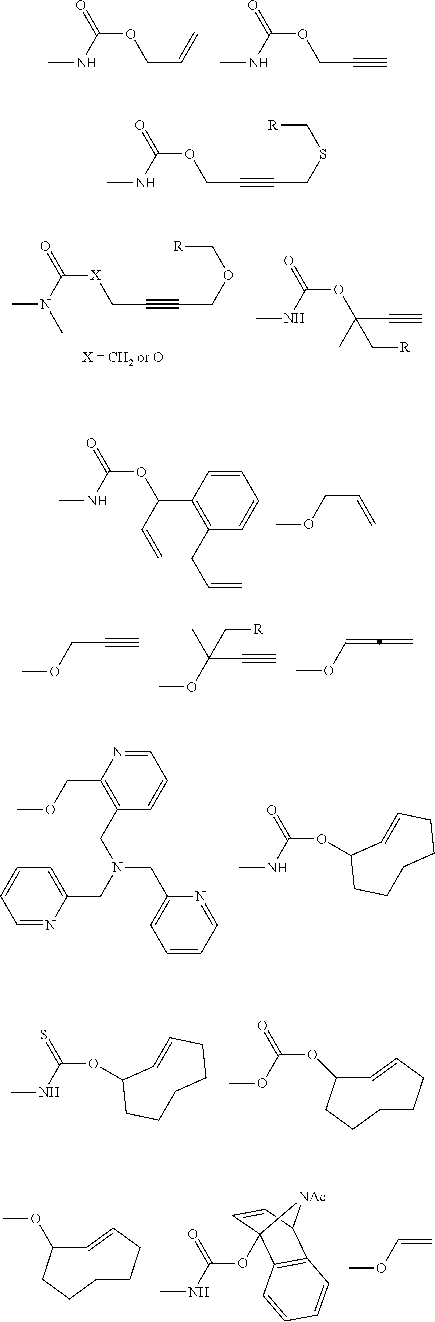

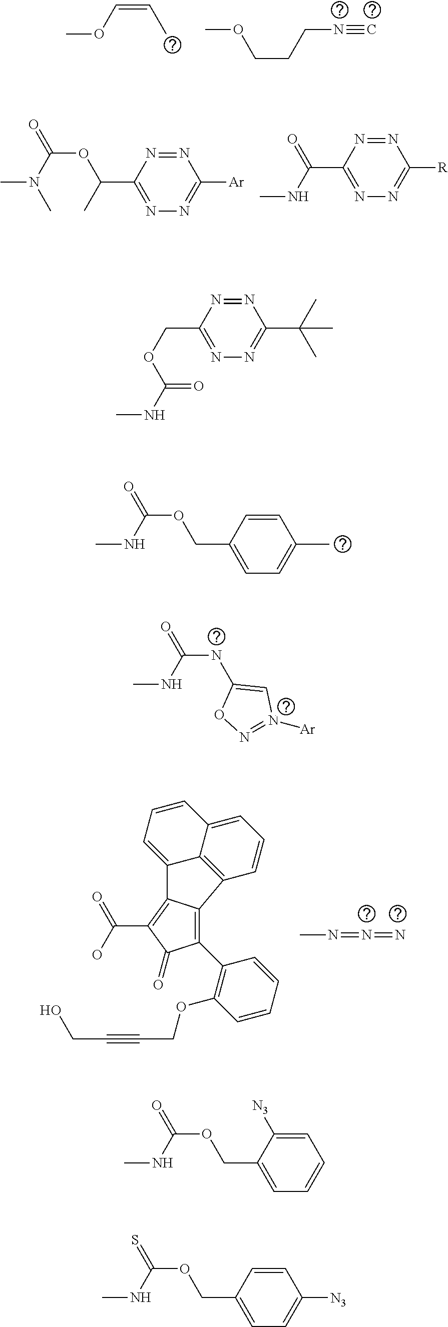

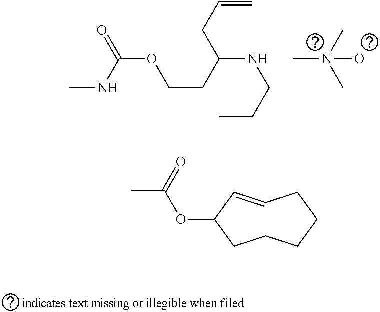

and any combination thereof.

In one embodiment the method of utilization is exemplified by but not limited to caging a first non-functional protein fragment to provide the caged reactive first protein fragment, and providing a complementary optionally-caged reactive second protein fragment; further comprising cleaving a functional protein at a predetermined location to provide a first non-functional protein fragment and a complementary reactive second protein fragment.

Self-assembling protein constructs have applications in by drogel matrices. In some nonlimiting embodiments a self-assembling protein construct can be a hydrogel material, comprising a caged reactive first protein fragment covalently bonded to a hydrogel matrix comprising a first stimulus-responsive cleavable moiety capable of cleaving from the caged reactive first protein fragment, upon application of a predetermined first stimulus, to provide a reactive first protein fragment; a first split protein linked with the caged reactive first protein fragment; a complementary reactive second protein fragment that is optionally bonded to the hydrogel matrix capable of reacting with the first reactive protein fragment; and a second split protein linked with the complementary reactive second protein fragment, wherein the first reactive protein fragment is adapted to react with the complementary reactive second protein fragment to provide a self-assembled protein or a portion thereof; and wherein the first split protein is adapted to associate with the second split protein and to form an active protein bonded to the hydrogel network in accordance with the reaction of the first reactive protein fragment and the complementary reactive second protein fragment providing the self-assembled protein or the portion thereof.

In some nonlimiting embodiments the self-assembling protein construct hydrogel can comprise of various types of water soluble polymeric In some nonlimiting examples the hydrogel material comprises of polyethylene glycol, polypropylene glycol, polyethylene glycol-co-polypropylene glycol, polyethylene glycol diacrylate, glutaminamide-modified polyethylene glycol, poly(lysine-phenylalanine) peptides, polyethylene glycol dimethacrylate, polyethylene glycol diacrylamide, polyethylene glycol dimethacrylamide, polyvinyl alcohol, cellulose, carboxy methylcellulose, methyl cellulose, hydroxyethyl cellulose, acrylic acid, acrylic acid sodium salt, a salt of acrylic acid, methacrylic acid, methacrylic acid sodium salt, a salt of methacrylic acid, polyvinyl pyrrolidone, polyvinyl sulfonic acid, polyvinyl sulfonic acid sodium salt, a salt of polyvinylsulfonic acid, polyvinylphosphonic acid, polyvinylphosphonic acid sodium salt, a salt of polyvinylphosphonc acid, starch, inulin, fructooligiosaccharide, oligofructose, polydextrose, xanthan gum, locust bean gum, gum Arabic, guar gum, hyaluronan, pectin, gelatin, carrageenan, alginate, sodium alginate, and any combination thereof.

The hydrogel material in some nonlimiting embodiments, the polymer material can be linear, branched, linear and branched and has a molecular weight of at least 1,000,000.

The hydrogel material in some nonlimiting embodiments, the polymer material can be a thermosetting crosslinked network.

Various types of reactions can be used to either form or crosslink the hydrogel material and some nonlimiting examples of reactions that can be used to yield the hydrogel material are azide alkyne cycloaddition, azide alkene cycloaddition, group transfer polymerization, 2+2 cycloaddition, 4+2 cycloaddition, 4+4 cycloaddition, 6+2 cycloaddition, 6+4 cycloaddition, Huisgen 1,3-dipolar cycloaddition, epoxy, ring opening polymerization, esterification, amidation, thiol-ene reaction, thiol-yne reaction, radical chain polymerization, Michael addition, polymerization free of radicals and any combination thereof.

In certain embodiments the hydrogel material has pSC-cUnaG (SEQ ID No. 39) covalently bonded to the said hydrogel material.

In some embodiments the hydrogel material has a protein covalently bonded to the hydrogel matrix and some nonlimiting examples are pSC-cUnaG (SEQ ID No. 39) is covalently bonded to the said hydrogel material by means of azide alkyne cycloaddition, azide alkene cycloaddition, group transfer polymerization, 2+2 cycloaddition, 4+2 cycloaddition, 4+4 cycloaddition, 6+2 cycloaddition, 6+4 cycloaddition, Huisgen 1,3-dipolar cycloaddition, epoxy, ring opening polymerization, esterification, amidation, thiol-ene reaction, thiol-yne reaction, radical chain polymerization, Michael addition, polymerization free of radicals and any combination thereof.

In some embodiments the hydrogel material has nUnaG-ST (SEQ ID No. 31) contained within the said hydrogel material or is covalently bonded to the said hydrogel material.

In some embodiments the stimulus-responsive cleavable moiety within the hydrogel is responsive to single photon processes.

In some embodiments the stimulus-responsive cleavable moiety within the hydrogel is responsive to two-photon processes.

In some embodiments the stimulus-responsive cleavable moiety within the hydrogel is responsive to single photon processes and displays no luminescence before application of said stimulus and luminescence after exposed to said stimulus.

In some embodiments the stimulus-responsive cleavable moiety in the hydrogel is responsive to two-photon processes and displays no luminescence before application of said stimulus and luminescence after exposed to said stimulus.

In some embodiments the stimulus-responsive cleavable moiety in the hydrogel is responsive to single photon processes and displays luminescence before application of said stimulus and no luminescence after exposed to said stimulus.

The following Examples describe LASL, its methods, analysis, and its applications in protein constructs.

### EXAMPLES

The following presents a system of an amino acid, a photoliable cage or stimulus responsive cage, and an irreversible conjugation of recombinant proteins or Spatiotemporal Functional Assembly of Split Protein Pairs through a Light-Activated SpyLigation (LASL) and applications of LASL thereof.

Chemical reagents and solvents were supplied by either Sigma-Aldrich or Fisher Scientific. Solvents were removed under reduced pressure using a Büchi Rotovapor R-3 equipped with a V-700 vacuum pump, V-855 vacuum controller, and a Welch 1400 DuoSeal Belt-Drive high vacuum pump. ¹H nuclear magnetic resonance (NMR) data was collected using Bruker instruments at 298 K and chemical shifts were determined relative to tetramethylsilane (TMS, δ=0). Synthesized species were lyophilized using the LABCONCO FreeZone 2.5 Plus with a LABCONCO rotary vane 117 vacuum pump. Small-molecule mass spectrometry data was collected by direct-injection into a Thermo Linear Trap Quadrupole Orbitrap Xcalibur 2.0 DS. Light exposures were performed using a Lumen Dynamics OmniCure S1500 Spot UV Curing system with an internal 365 nm filter, collimator, and an external 360 nm cut-on longpass filter. Light intensity was measured using a Cole-Parmer Series 9811-50 Radiometer (λ=365 nm). Gradient patterns were created with a Harvard Apparatus PHD 2000 Syringe Pump outfitted with an opaque mask. Polymerase chain reaction (PCR) was achieved using a Bioer LifeECO thermo cycler. E. coli cultures were maintained in a Thermo Scientific MaxQ 4000 shaker incubator. Cell lysis was achieved with a Fisher Scientific Model 505 Soni Dismembrator equipped with a 1.27 cm diameter probe. Nucleic acid (λ=260 nm) and protein (λ=280 nm) concentrations were measured with a NanoDrop (Thermo Scientific). Protein fluorescence was quantified using a BioTek Synergy H1M plate reader. Fluorescence excitation/emission spectra were obtained on a Horiba Fluorlog®-3 (FL3-21tau) Fluorescence Spectrophotometer. Protein gel electrophoresis was executed with a Mini-PROTEAN tetra gel box equipped with a PowerPac basic power supply (BioRad). Fluorescence imaging was performed on a Leica Stellaris 5 confocal microscope equipped with a white light laser, live imaging chamber, and 10×CS2 APO dry objective. Bioluminescence assays were performed on a BioTek Synergy H1M plate reader. Fluorescent, luminescent, and true color gel imaging was performed on an Azure 600 AZI600 scanner. Multiphoton lithography was performed on a Thorlabs Bergamo II multiphoton microscope equipped with a Coherent Chameleon Discovery NX laser and an Olympus water-immersion objective (25×, NA=0.95).

### Example 1

### Construction of a Photoactivatable SpyCatcher (pSC. SEQ ID No. 26) for LASL

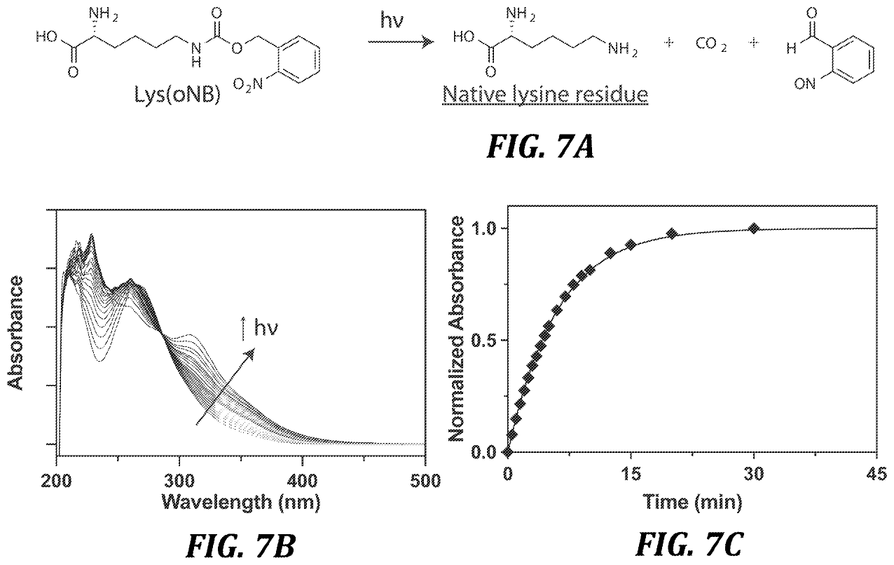

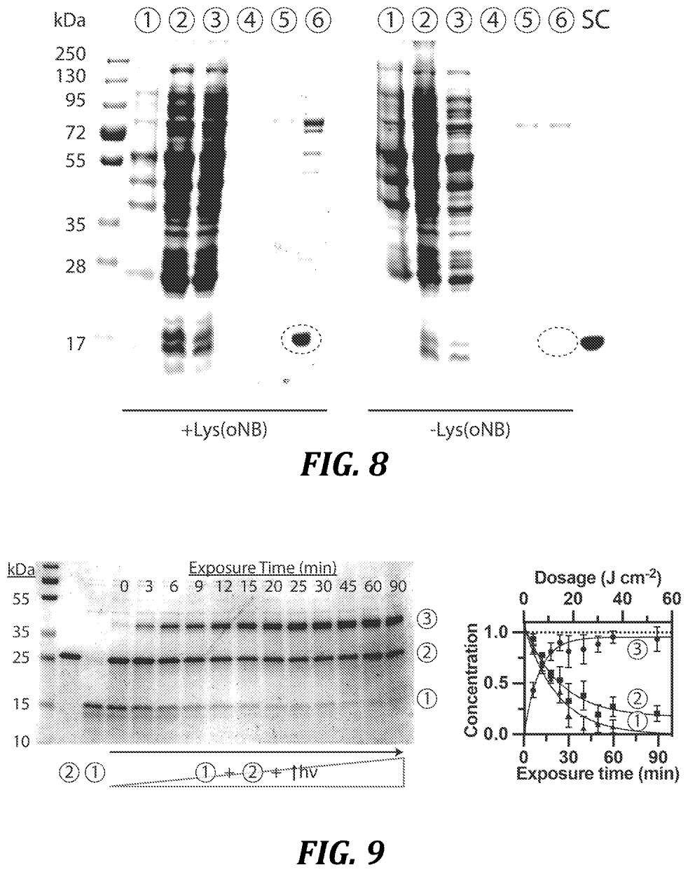

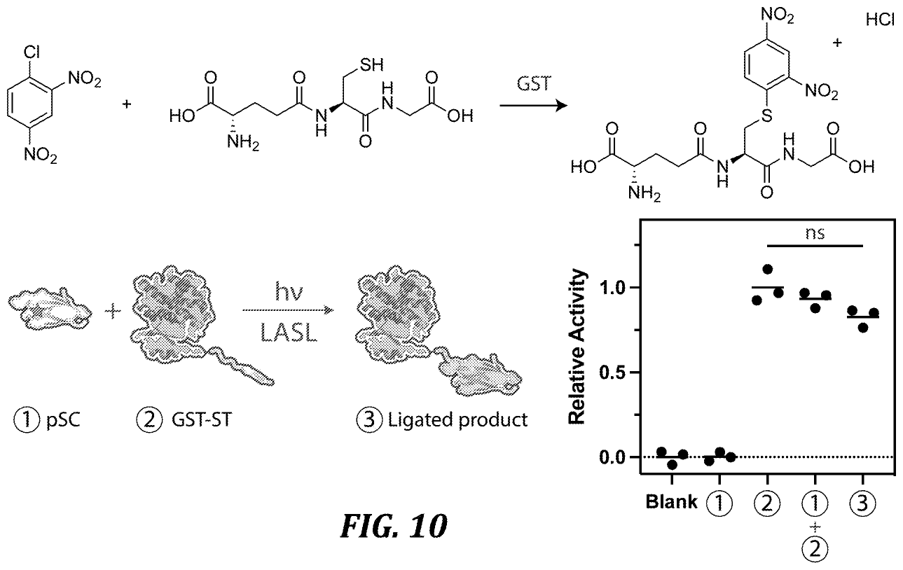

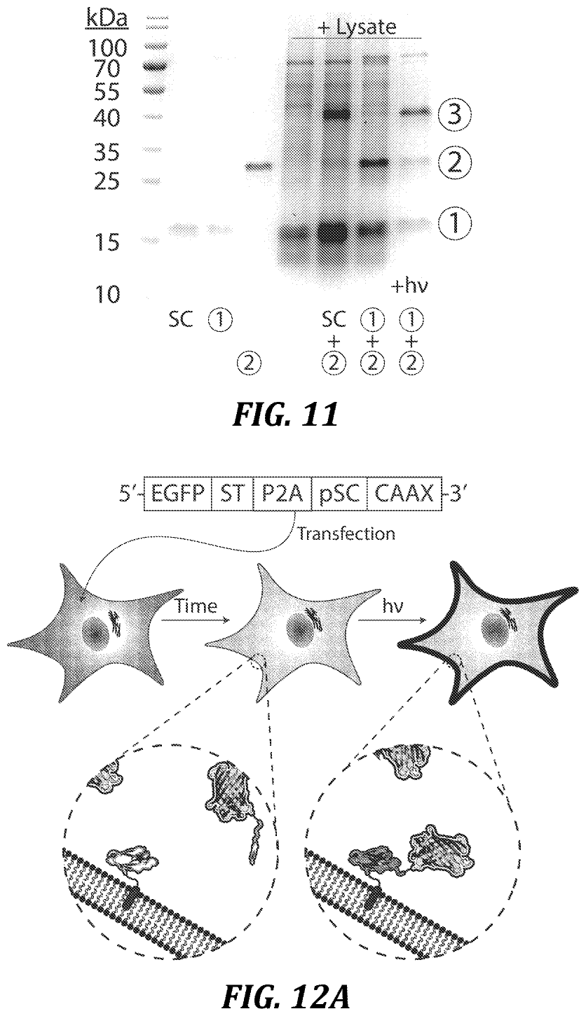

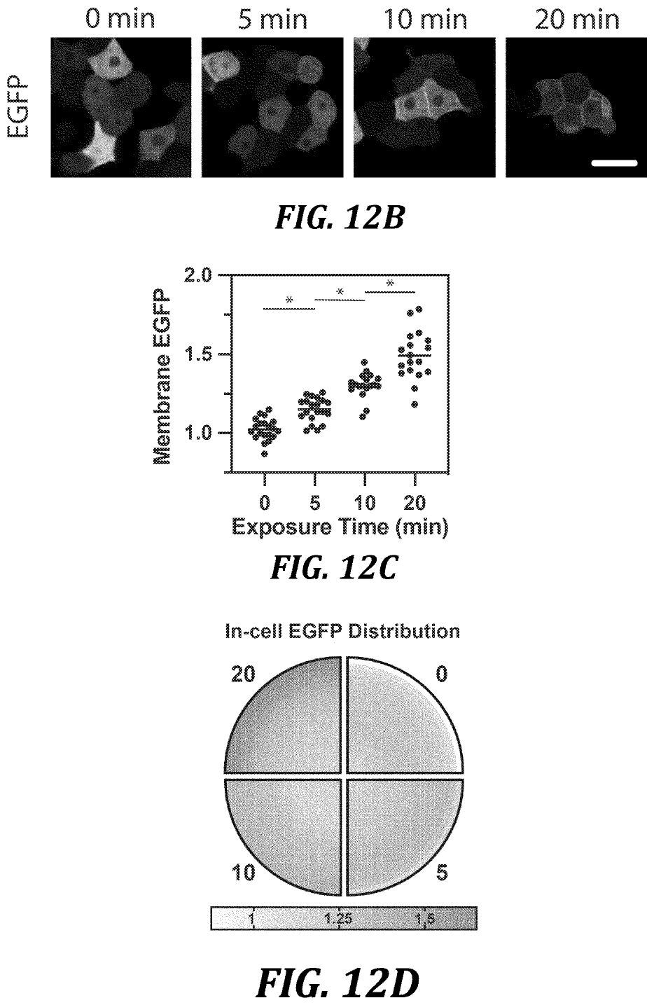

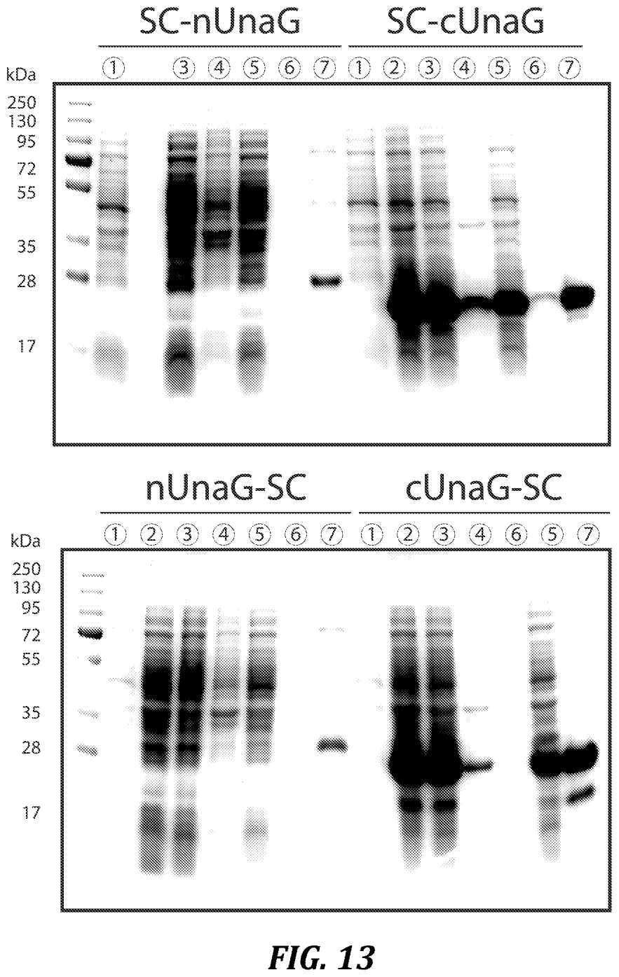

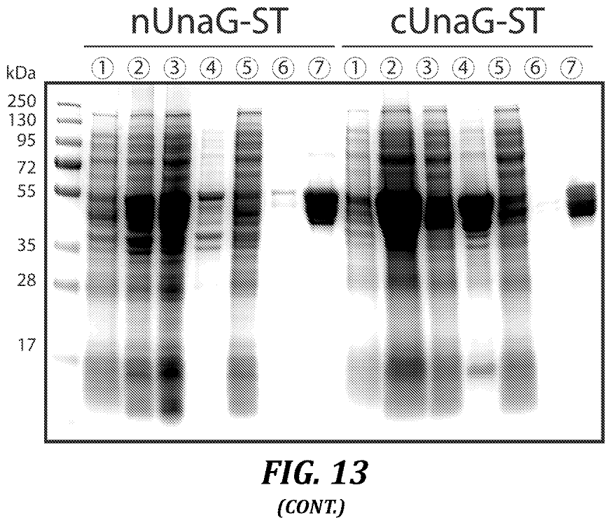

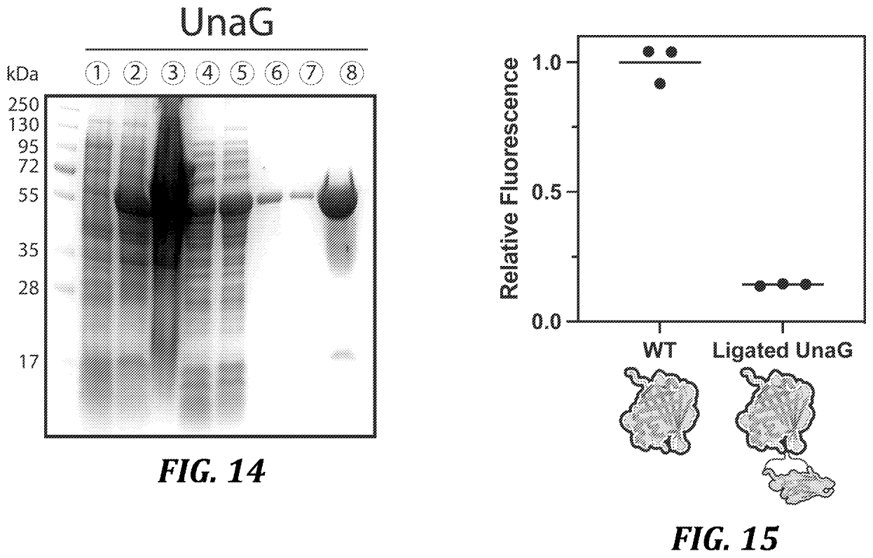

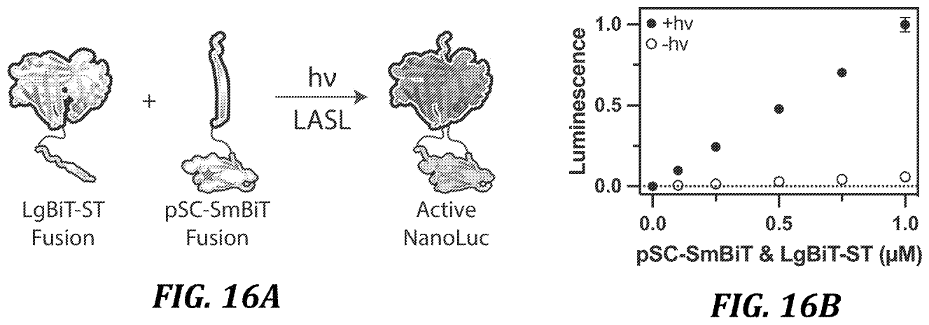

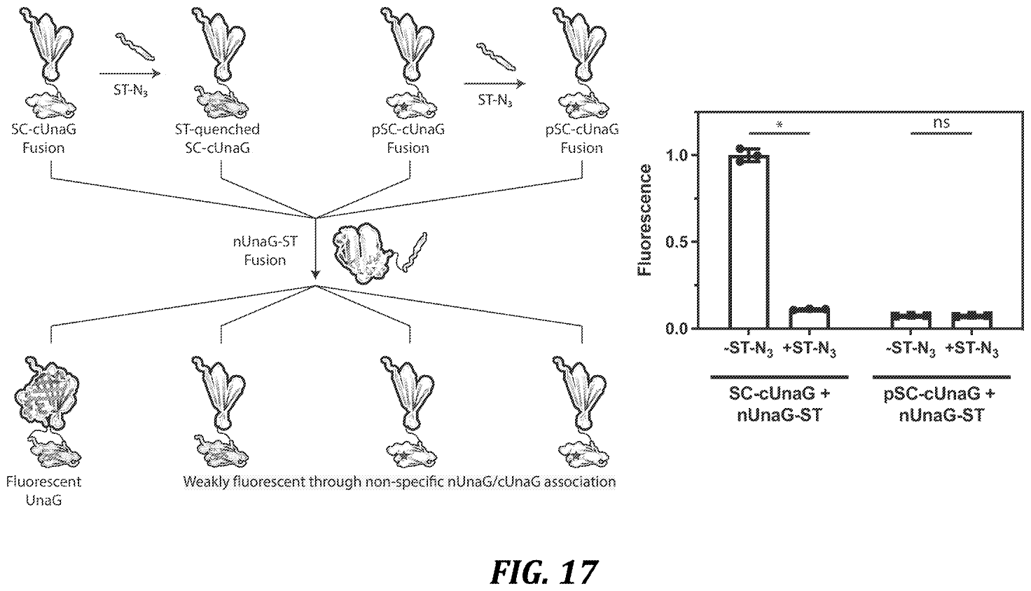

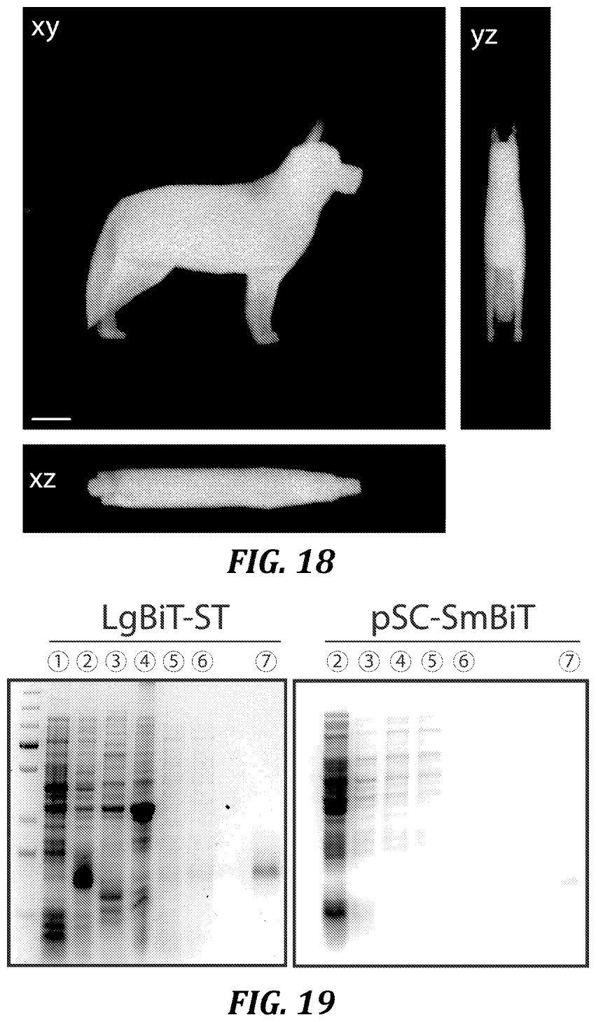

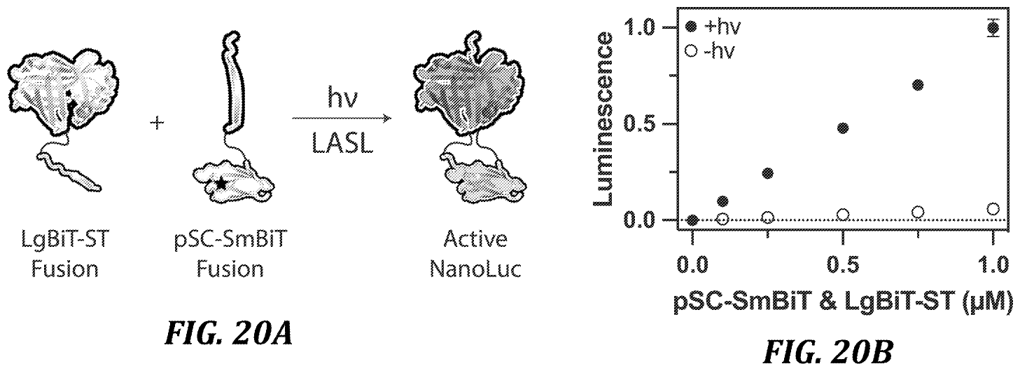

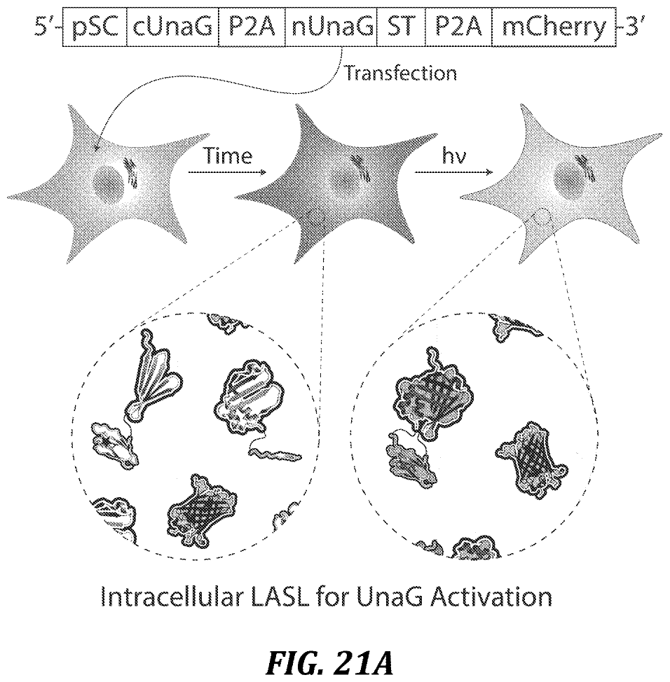

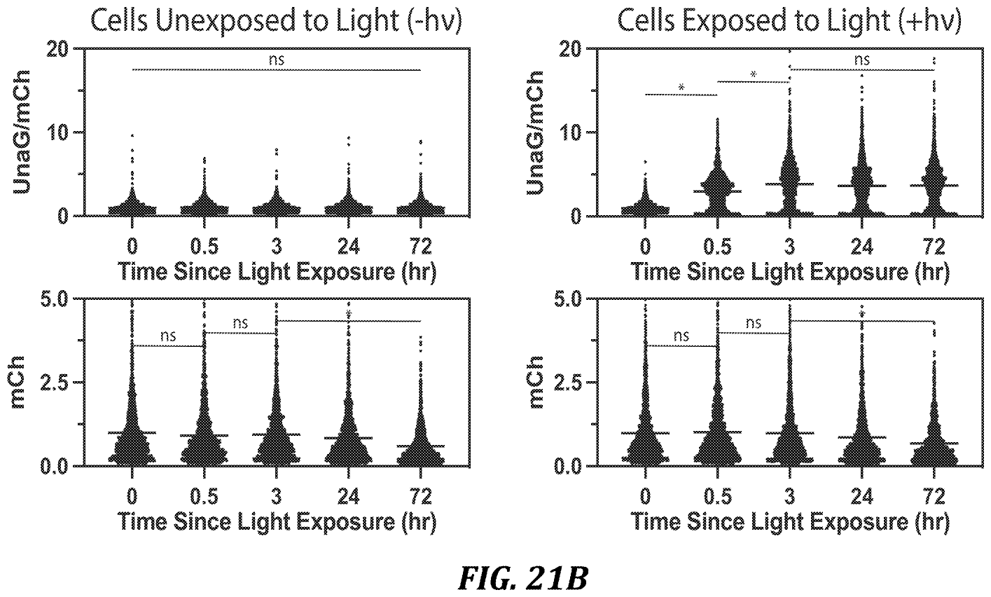

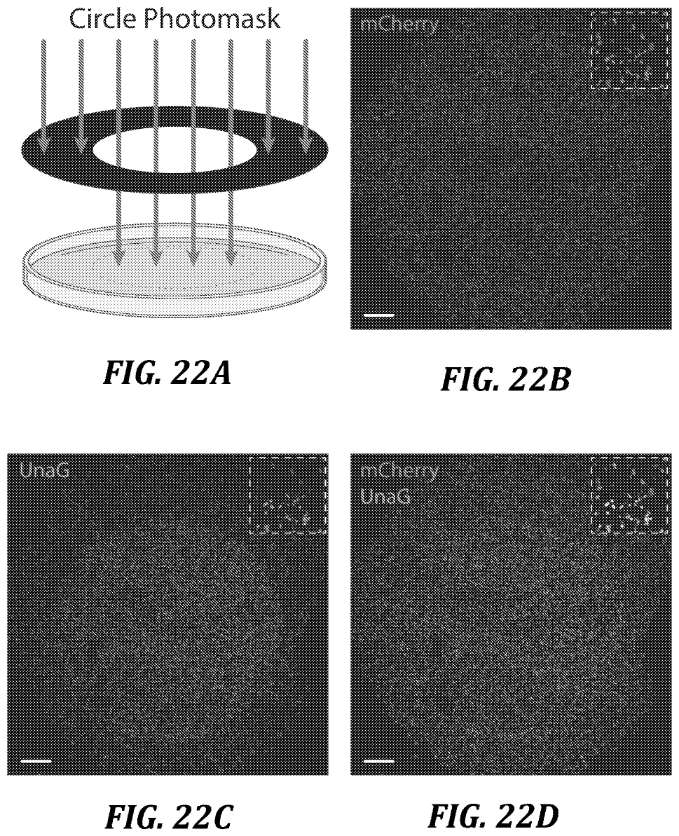

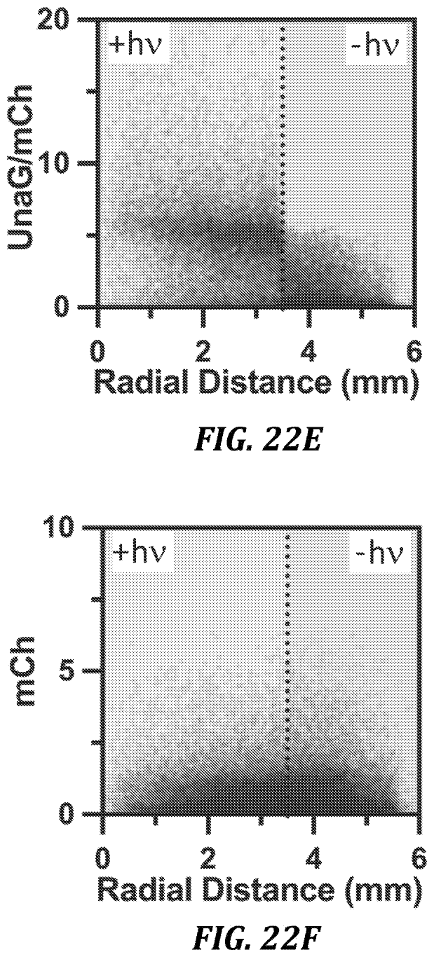

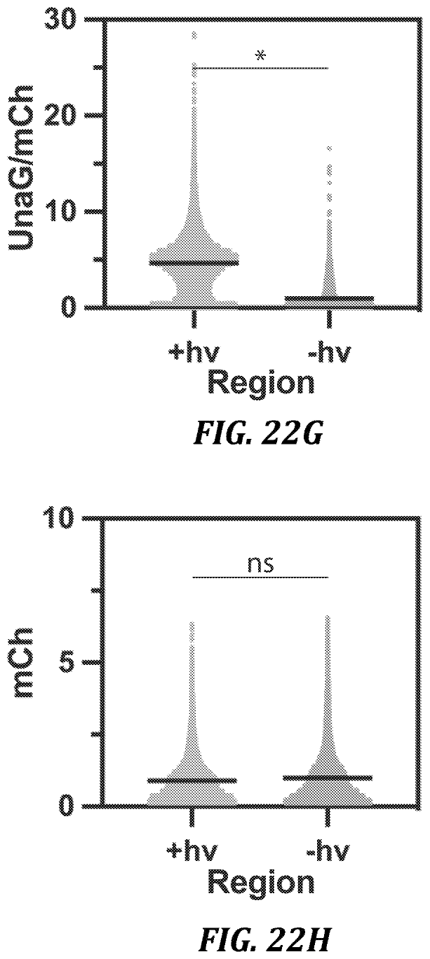

Success of the LASL strategy hinges upon efficient incorporation of Lys(oNB) within pSC (SEQ ID No. 26), necessitating a high-yielding synthesis of the photocaged lysine and effectively engineered orthogonal aminoacyl-tRNA synthetase (aaRS)/tRNA pairs for site-specific non-canonical amino acid installation in both bacterial and mammalian systems. Lys(oNB) was produced through an improved synthetic route, yielding sufficient gram quantities of the caged amino acid for several liters of protein expression from a single two-step synthesis (EXAMPLE 2 Method S1). Upon in-solution light exposure and oNB photocage removal, native lysine was recovered following expected first-order photocleavage kinetics (FIG. 7). For bacterial expression, we constructed a plasmid encoding for two copies of a mutant Methanosarcina maize pyrrolysyl-tRNA synthetase (MmPylRS; Y306M, L309A, C348A, Y384F) previously evolved for Lys(oNB) incorporation and a single copy of its cognate Pyl-tRNAcnIA (EXAMPLE 2 Method 2). To install Lys(oNB) site-specifically in mammalian cells, we utilized a plasmid encoding for a mutant Methanosarcina bakeri synthetase MbPylRS; Y271A. Y349F) compatible with derivatized lysine species—but not previously with Lys(oNB)—and four copies of an engineered M15-tRNA_{CUA} recently reported to boost incorporation of non-canonical amino acids in pyrrolysine-based systems (EXAMPLE 2 Method 2). Employing the relevant aaRS/tRNA plasmid for genetic code expansion in bacteria or mammalian cells, pSC (SEQ ID No. 26) was created through co-translational incorporation of Lys(oNB) at SC's (SEQ ID No. 25) catalytic Lys31 residue via amber suppression (EXAMPLE 2 Method S3).

### Validation and Characterization of LASL Using Purified Proteins

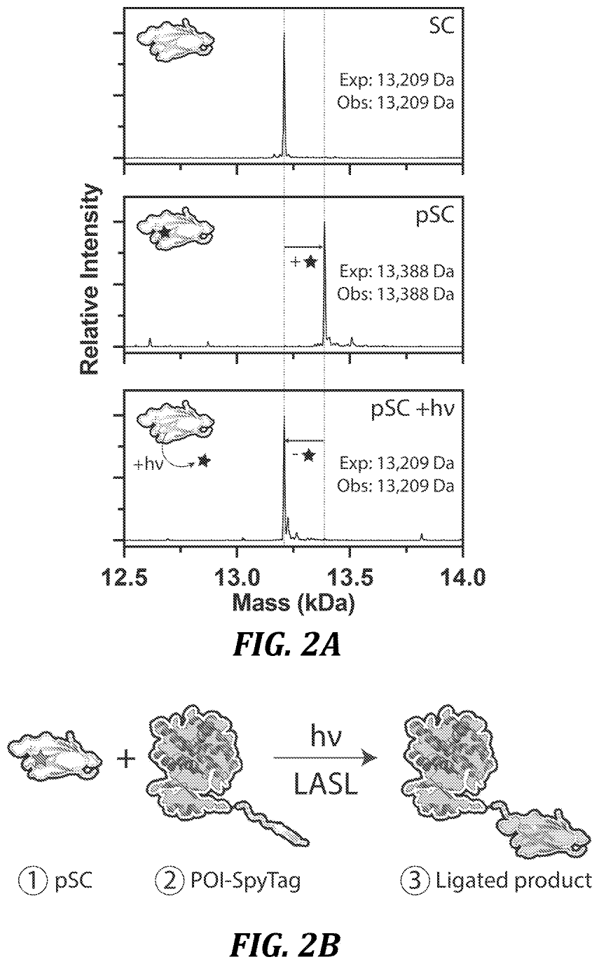

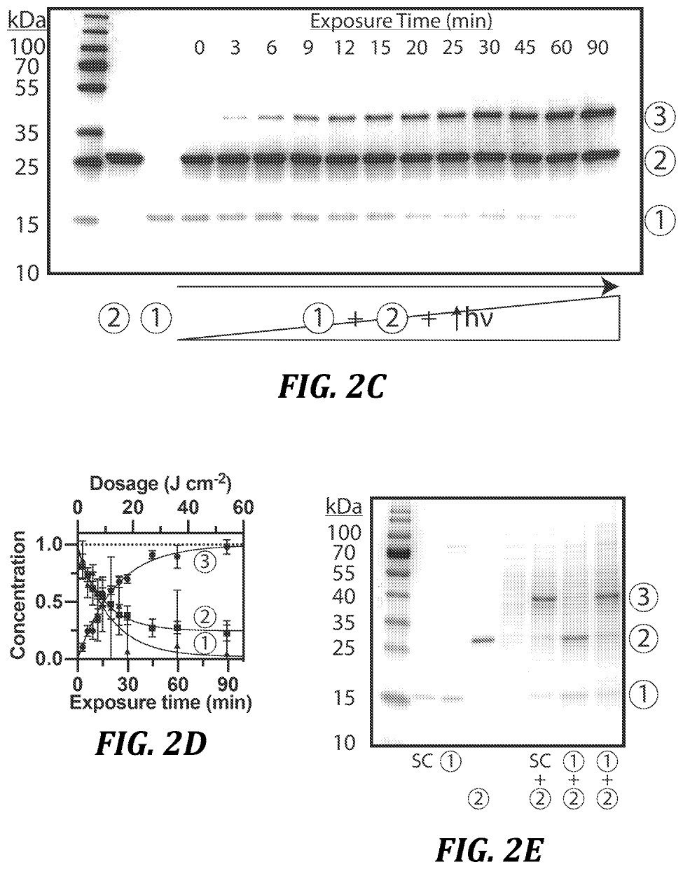

To assess and quantify LASL's efficiency in vitro, we recombinantly expressed pSC (SEQ ID No. 26), SC (SEQ ID No. 25), and a glutathione S-transferase-SpyTag fusion (GST-ST (SEQ ID No. 28)) in E. coli and purified each species via affinity chromatography (EXAMPLE 2 Methods S4-S5). Highly pure samples with the expected molecular masses were obtained for all proteins, as indicated by sodium dodecyl sulfate-polyacrylamide gel electrophoresis (SDS-PAGE) analysis and through liquid chromatography-mass spectrometry (LC-MS)(FIG. 8, EXAMPLE 2 Method 6, EXAMPLE 2 Table 1). When exposed to near-UV light (λ=365 nm, 10 mW cm, 30 min), pSC's (SEQ ID No. 26) oNB cage (delta mass=179 Da) was photochemically removed to yield protein that precisely matched the mass of the native SC construct (13.2 kDa)(FIG. 2A). To assess whether this photoproduct was functional and could participate in SpyLigation, we reacted differentially light-exposed pSC (SEQ ID No. 26) (λ=365 nm, 10 mW cm². 0-90 min) with excess GST-ST (SEQ ID No. 28) at physiological temperature (37° C.) for non-kinetically limited times (18 h) prior to SDS-PAGE analysis (FIG. 2B-2C, EXAMPLE 2 Method S7). The extent of photoligation was determined by quantifying the intensities of the disappearing bands from pSC (SEQ ID No. 26) and GST-ST (SEQ ID No. 28) along with the appearing band corresponding to the SpyLigated product (FIG. 2D). These analyses revealed a first-order photouncaging constant of 0.05±0.01 min⁻¹ and a half-life of 15±2 min, respectively corresponding to 0.07±0.02 cm⁻² J⁻¹ and 9±1 J cm⁻² when accounting for the utilized light intensity. Uncaging photokinetics remained unchanged when pSC (SEQ ID No. 26) was exposed to light in the presence of GST-ST (SEQ ID No. 28), and GST-ST (SEQ ID No. 28) bioactivity was unperturbed upon incubation and subsequent photoligation with pSC (SEQ ID No. 26, FIG. 9-10). Highly satisfied with the observed near-complete ligation in optically stimulated samples and an absence of “dark reaction” for unexposed pSC (SEQ ID No. 26), we were encouraged to test LASL's efficiency in more complex environments. When performed in E. coli or human embryonic kidney 293T (HEK-293T) mammalian cell lysate, LASL yielded the expected ligated product band with seemingly no unintended reactions with other protein components (FIG. 2E, EXAMPLE 2 Methods S8-S9, FIG. 11). Collectively, these experiments establish that LASL is photocontrollable with high specificity and in a dosage-dependent manner.

Spatial Control Over LASL within Hydrogel Biomaterials

Photochemical reactions are unique in that they can be spatiotemporally initiated based on when and where light is directed onto reactants. This feature is now regularly exploited in many subfields, including by the biomaterials community to engineer cell-culture platforms with user-defined and heterogeneous biochemistry. To guide complex anisotropic cellular functions in vitro, our lab and others have utilized photochemistry to pattern full-length protein immobilization within polymeric hydrogels whose stiffness, water content, and other essential features mimic those of native tissue. Though recent efforts have demonstrated the importance of site-specific protein modification in maintaining their bioactivity upon tethering, genetically encoded photochemistries to control such immobilization have not yet been established. Towards filling this gap, we opted to employ LASL in the photopatterning of poly(ethylene glycol) (PEG)-based hydrogels formed througal strain-promoted azide-alkyne cycloaddition (SPAAC).

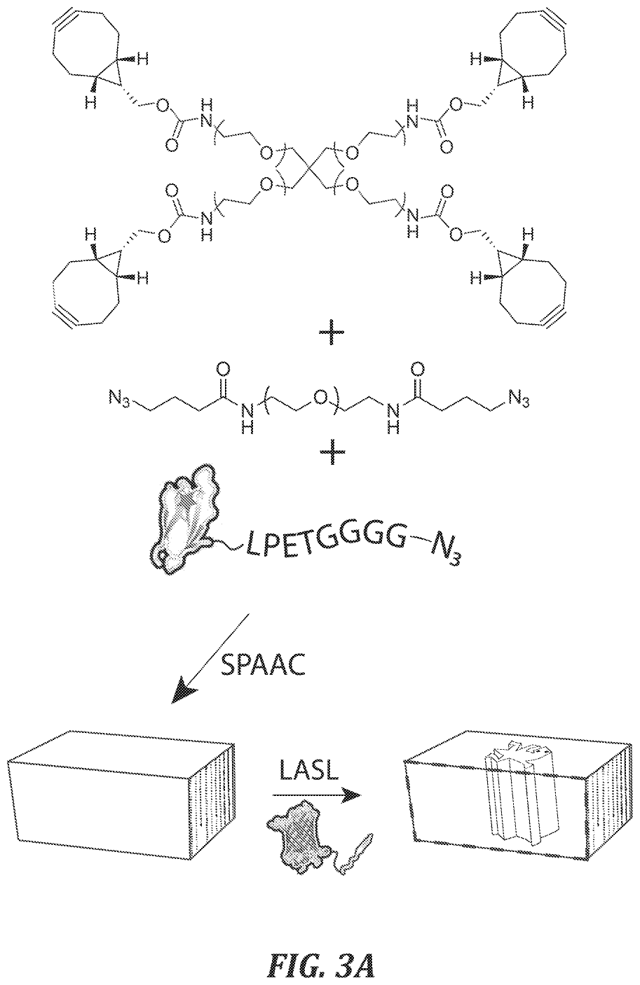

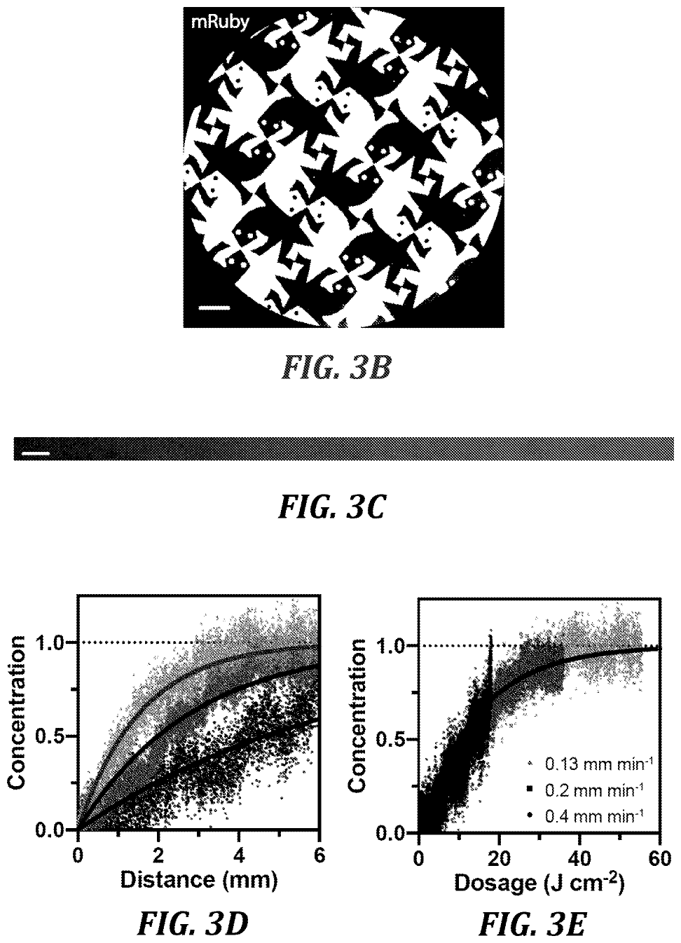

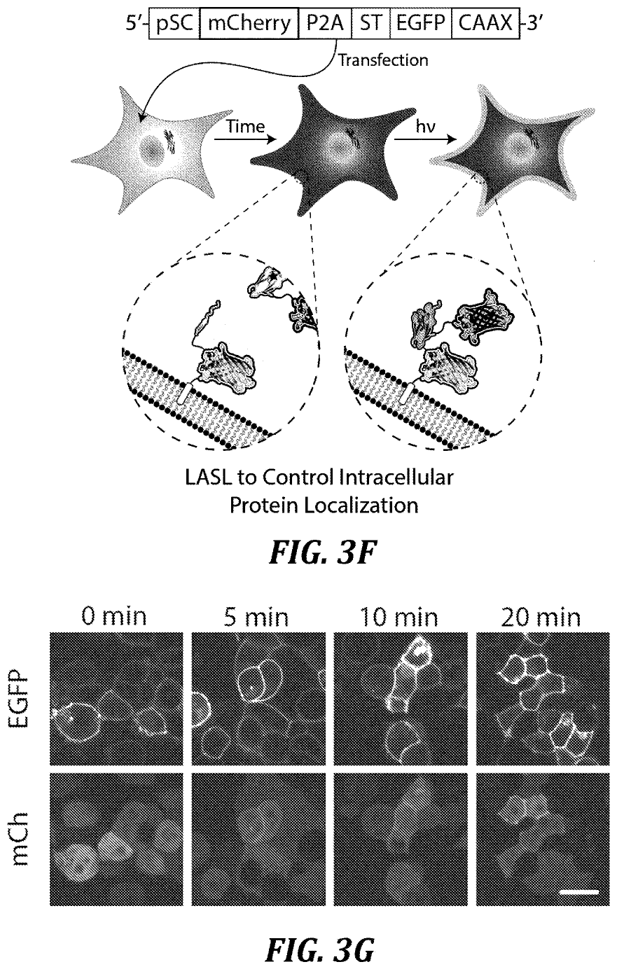

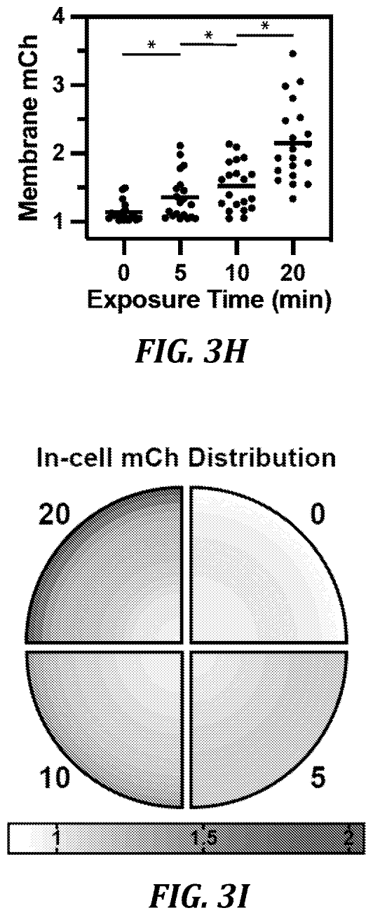

To immobilize the photoactivatable SpyCatcher uniformly within SPAAC gels, a pSC (SEQ ID No. 26) variant containing a C-terminal sortase recognition motif (i.e., LPETG) was expressed, purified, and chemoenzymatically modified with an azido-polyglycine peptide probe [H-SEQ ID No. 50(N₃)—NH₂] to yield the azide-monotagged pSC (SEQ ID No. 26, pSC-N₃) (EXAMPLE 2 Method S10). Photocaged SpyCatcher-decorated gels were formed through step-growth polymerization of PEG tetrabicyclononyne (PEG-tetraBCN, Mₙ˜20 kDa), linear PEG-diazide (Mₙ˜3.5 kDa), and pSC-N₃ (SEQ ID No. 26. FIG. 3A, EXAMPLE 2 Method S11). Upon mild near-UV irradiation, the oNB cage is cleaved, converting pSC (SEQ ID No. 26) into its active form and permitting localized conjugation with gel-swollen SpyTagged (SEQ ID No. 7, SEQ ID No. 8) proteins via LASL. Diffusive removal of unbound proteins yields patterned gel substrates defined by user-selected light-exposure locations and parameters. Gel modification was performed with a SpyTagged mRuby (mRuby-ST (SEQ ID No. 29). EXAMPLE 2 Method S4), whereby red fluorescence of the immobilized protein permitted visualization and quantification. Traditional photolithographic techniques were utilized to control patterning of mask-defined shapes throughout gels with single micron-scale resolution; no background fouling or undesired conjugation was observed, highlighting minimal association of ST with pSC (SEQ ID No. 26, FIG. 3B). Linear exposure gradients imposed across the gel surface using an opaque photomask moving at variable rates (0.13, 0.2, 0.4 mm min-) yielded continuous exponential protein gradients of predicted shape (FIG. 3C, FIG. 3D). When accounting for gel position and mask translational speed, gradients collapsed onto a single dosage-response curve that revealed a pSC (SEQ ID No. 26) uncaging kinetic constant of 0.06±0.02 cm² J⁻¹ and a half-life of 11±2 J cm⁻², both statistically indistinguishable from values determined in solution (FIG. 3E). These experiments represent the first demonstration of a genetically encoded photochemistry to immobilize proteins site-specifically within materials, as well as highlight LASL's compatibility with other state-of-the-art protein modification chemistries and its ability to be performed with high spatiotemporal control.

Spatial Control Over LASL within Living Mammalian Cells

Building upon our success in spatially controlling biomacromolecular tethering within hydrogel biomaterials, we turned our efforts towards optically specifying protein subcellular location within living mammalian cells using LASL. Here, we cloned a polycistronic construct consisting of a pSC-tagged mCherry (mCh), a “self-cleaving” P2A peptide sequence, and an Enhanced Green Fluorescent Protein (EGFP) fused with ST and a plasma membrane-localizing CAAX motif from K-Ras (EXAMPLE 2 Method S12, FIG. 3F). HEK-293T cells were co-transfected with plasmids encoding for the membrane labeling components and the mutant MbPylRS/tRNA pair (EXAMPLE 2 Method 13). When cultured in media supplemented with Lys(oNB), cells fluoresced red (mCh) throughout their cytosol and green (EGFP) at their plasma membranes, indicating successful read-through of pSC's (SEQ ID No. 26) in-frame amber stop codon and EGFP-ST membrane targeting with the CAAX motif (FIG. 3H). Following flood illumination (λ=365 nm, 20 mW cm², 0-20 min), the uncaged pSC-mCh colocalized with EGFP-ST at the plasma membrane via LASL. As predicted, membrane labeling with and subcellular distribution of pSC-mCh scaled in a statistically significant manner with light dosage (FIG. 3H-3I). Complementary experiments in which a membrane-bound pSC (SEQ ID No. 26) was phototagged with EGFP-ST yielded similar results (EXAMPLE 2 Methods S12-S13, FIG. 12). Together, these studies showcase LASL's ability to efficiently specify lasting changes to intracellular protein localization in a dose-dependent manner.

### Split Protein Assembly and Functional Activation Using LASL

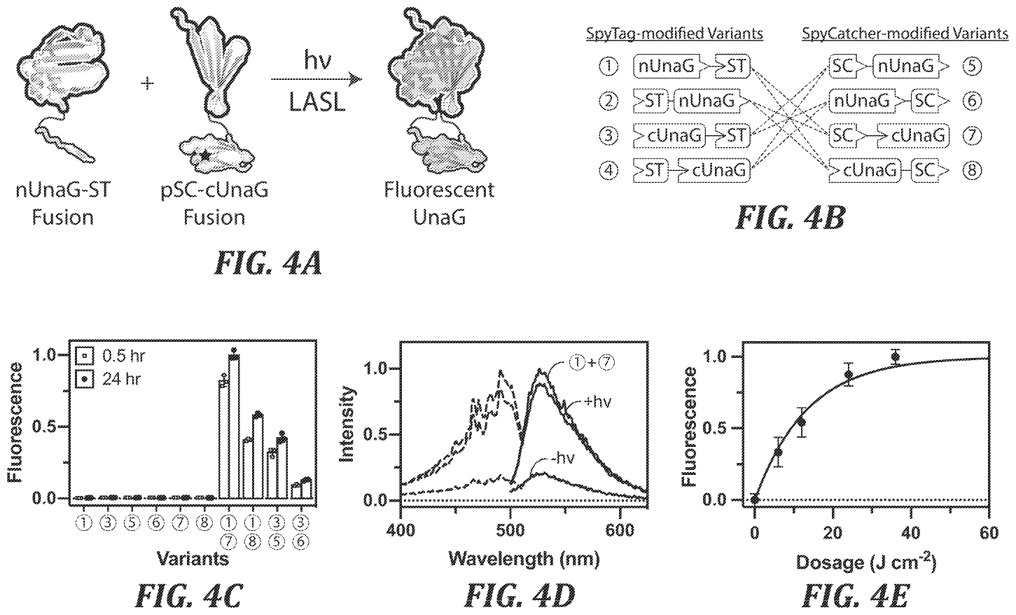

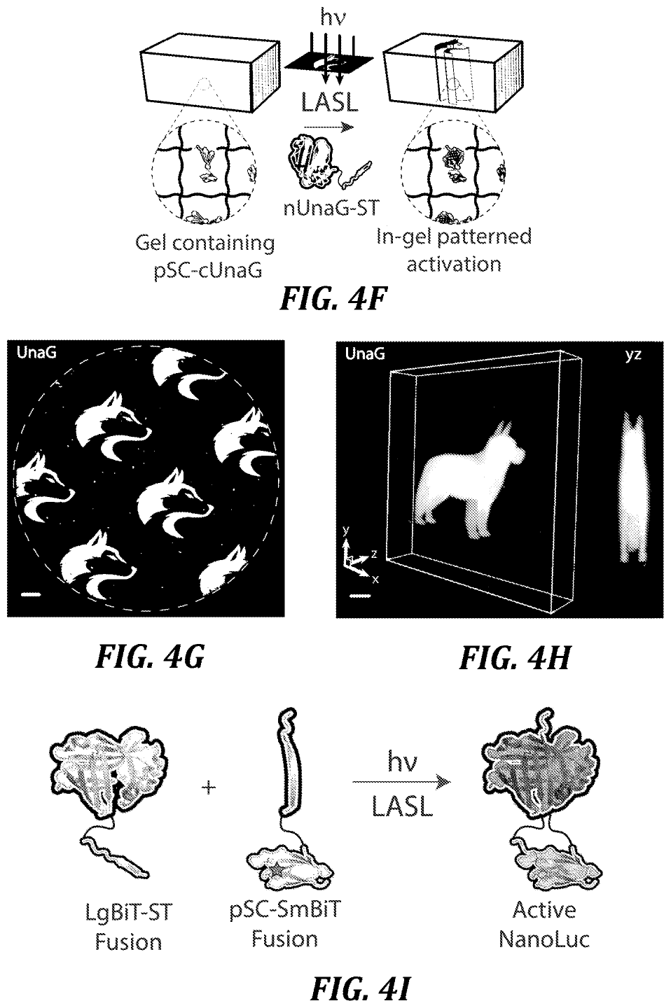

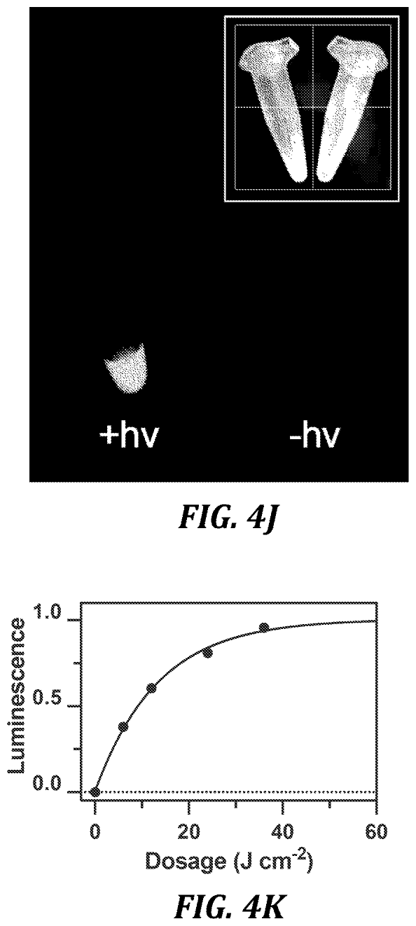

Motivated by our unique ability to photoligate genetically encoded protein pairs with spatiotemporal control using LASL, we next sought to utilize the reaction to irreversibly restore protein function through covalent ligation of otherwise inactive split proteins. As an initial proof of concept, we selected UnaG (SEQ ID No. 41), a green fluorescent protein derived from Japanese eel muscle whose activity can be optically assessed rapidly at single- and sub-cellular resolutions (FIG. 4A). Since its fluorogenic chromophore, bilirubin, is non-covalently bound and readily available in sera and in vivo, UnaG (SEQ ID No. 41) exhibits virtually no long-term photobleaching in living systems; having an optical readout for protein function that would not be affected by LASL's requisite light exposure led us to explore UnaG (SEQ ID No. 41) over alternative fluorescent proteins. Recognizing that split protein functional activation is dependent on factors often difficult to predict a priori (e.g. geometry, sterics, intermediate folding), we exploited a previously validated split site for rapamycin-induced UnaG (SEQ ID No. 41) assembly (nUnaG=residues 1-84; cUnaG=residues 85-139) and cloned bacterial expression vectors for all possible permutations wherein fragments were N- or C-terminally monofunctionalized with either ST or wild-type SC (FIG. 4B, EXAMPLE 2 Method S14). 6×His-tagged proteins were expressed in E. coli and purified by immobilized metal-ion affinity chromatography. Variants containing N-terminal ST moieties did not readily express in a soluble form and were thus abandoned; all other species were successfully obtained in good yield, validated as pure (SDS-PAGE, FIG. 13), and determined to exhibit the expected molecular weight (LC-MS, EXAMPLE 2 Table 1). Though individual components lacked fluorescence. SpyLigated UnaG (SEQ ID No. 41) fragments brightly fluoresced after 30 min of reaction at levels approaching those from samples following extended incubation (24 h) (FIG. 4C. EXAMPLE 2 Method S15), consistent with the rapid kinetics previously reported for the native SpyLigation. Conjugation of nUnaG-ST (SEQ ID No. 31) and SC-cUnaG (SEQ ID No. 37) gave a fluorescent species that was between 2 and 5-fold brighter than all other fragment combinations, a result consistent with structural protein evaluation as ST's N-terminus is colocalized with the C-terminus of SC. SpyLigated nUnaG-ST (SEQ ID No. 31) and SC-cUnaG (SEQ ID No. 37) exhibited ˜15% maximal fluorescence compared with the wild-type UnaG (SEQ ID No. 41) species (EXAMPLE 2 Method S16, FIG. 14-FIG. 15); though reconstituted bioactivity depends just as much on the protein identity and split site as it does the dimerizing chemistry, this finding is consistent with the best reported inducible-dimerizing split protein systems.

After identifying nUnaG-ST (SEQ ID No. 31) and SC-cUnaG (SEQ ID No. 37) as the brightest split protein combination, we cloned, expressed, and purified the photocaged SC variant (pSC-cUnaG. SEQ ID No. 39) for LASL assembly (EXAMPLE 2 Method S14). To assess whether LASL could be used to photochemically restore UnaG (SEQ ID No. 41) activation, stoichiometrically matched nUnaG-ST (SEQ ID No. 31, 0-10 μM) and light-exposed pSC-cUnaG (SEQ ID No. 39, λ=365 nm, 20 mW cm⁻², 0-30 min) were combined (EXAMPLE 2 Method S17). Sample fluorescence was measured immediately following light exposure and again after 24 I. Fluorescence excitation/emission spectra for the LASL product following the longest exposures (30 min) matched that of the spontaneous SpyLigation (i.e., nUnaG-ST (SEQ ID No. 31) with SC-cUnaG, SEQ ID No. 37) and of wild-type UnaG (SEQ ID No. 41, FIG. 4D, EXAMPLE 2 Method S18). LASL-restored UnaG (SEQ ID No. 41) fluorescence scaled linearly with protein concentration and persisted well after initial light exposure (FIG. 16). Only small amounts of background fluorescence (˜15% maximum) were observed in unexposed samples, which we attributed to non-covalent association of the UnaG (SEQ ID No. 41) fragments partially stabilized with bilirubin (EXAMPLE 2 Methods S19-S20, FIG. 17). UnaG (SEQ ID No. 41) assembly/activation followed LASL's predicted dosage dependency (FIG. 4E): here, we observed a pSC (SEQ ID No. 26) uncaging kinetic constant of 0.07±0.02 cm⁻² J⁻¹ and a half-life of 10±2 J cm⁻² that was statistically indistinguishable from values determined in solution and in biomaterials.

Building on our ability to photoassemble UnaG (SEQ ID No. 41) in solution, we extended efforts towards spatiotemporally controlling its activation in 4D. Though studies reported earlier (FIG. 3) and elsewhere have demonstrated photochemical immobilization of full-length proteins within hydrogels, patterned functional assembly of split proteins in biomaterials has not been reported. Generation of active protein only at desired locations within gels offers distinct advantages over strategies involving diffusive transport of active proteins prior to patterned tethering, particularly if these proteins are to be used to guide embedded cell fate: the latter technique floods cells with active protein throughout the patterning process whereas the former would not. Towards this goal, we appended pSC-cUnaG (SEQ ID No. 39) with a C-terminal sortase recognition motif; this protein (pSC-cUnaG-LPETG, SEQ ID No. 40) was expressed, purified, and sortagged with the peptide H-SEQ ID No. 50(N₃)—NH₂ to yield monotagged pSC-cUnaG-N₃ (SEQ ID No. 39, EXAMPLE 2 Method S21). SPAAC-based gels modified uniformly with pSC-cUnaG-N₃ (SEQ ID No. 39) were formed through reaction of PEG-tetraBCN and a linear PEG-diazide (FIG. 4F, EXAMPLE 2 Method S22). When incubated with nUnaG-ST (SEQ ID No. 31) and exposed to photomasked light, UnaG (SEQ ID No. 41) activation was confined to 2D mask-defined shapes extending throughout the gel thickness (FIG. 4G). Multiphoton laser-scanning lithography, whereby programmed laser raster scanning within the gel specified photoactivation with full 3D control, afforded excellent 3D patterning at user-specified regions within the gel (FIG. 4H, FIG. 18).

Having shown that LASL can be used to irreversibly assemble and activate UnaG (SEQ ID No. 41), we sought to highlight the versatility of these methods through extension to another functional protein. We identified NanoLuc as a bioluminescent enzyme that has found great utility in its split form for quantifying protein-protein interactions. Adopting the optimal protein arrangement for LASL, identified structurally and with UnaG (SEQ ID No. 41, i.e., nPOI-ST+pSC-cPOI, where nPOI and cPOI refer to the N- and C-fragments of each protein of interest), we fused NanoLuc's N-terminal fragment (LgBiT, 159 amino acids, 17.6 kDa) with ST and its C—fragment (SmBiT, 11 amino acids, 1.6 kDa) to pSC (SEQ ID No. 26). LgBiT-ST (SEQ ID No. 43) and pSC-SmBiT (SEQ ID No. 42) were expressed in E. coli and purified by immobilized metal-ion affinity chromatography (FIG. 4I, EXAMPLE 2 Method S23, FIG. 19). When NanoLuc fragments were combined with equal stoichiometry (0-1 μM) in the presence of their substrate (furimazine) almost no luminescence (˜5% maximum) was observed; this absence of dark activity is consistent with minimal reported association of LgBiT and SmBiT as well as our findings that pSC (SEQ ID No. 26) and ST do not associate appreciably. Via LASL, protein bioactivity was restored in a light dose- and concentration-dependent manner following UV treatment (λ=365 nm, 20 mW cm⁻², 0-30 min) (FIG. 4J-4K, EXAMPLE 2 Method 24, FIG. 20) with uncaging kinetics (kinetic constant of 0.07±0.02 cm² J⁻¹; half-life of 9±2 J cm⁻²) matching those for mRuby immobilization, UnaG (SEQ ID No. 41) activation, and in-solution Lys(oNB) photolysis. Taken together, these results establish our ability to precisely and irreversibly photoactivate functional proteins through LASL-mediated split fragment reconstitution with micron-scale 3D control in a “plug-and-play” manner.

Spatiotemporally Controlled Split Protein Activation within Mammalian Cells

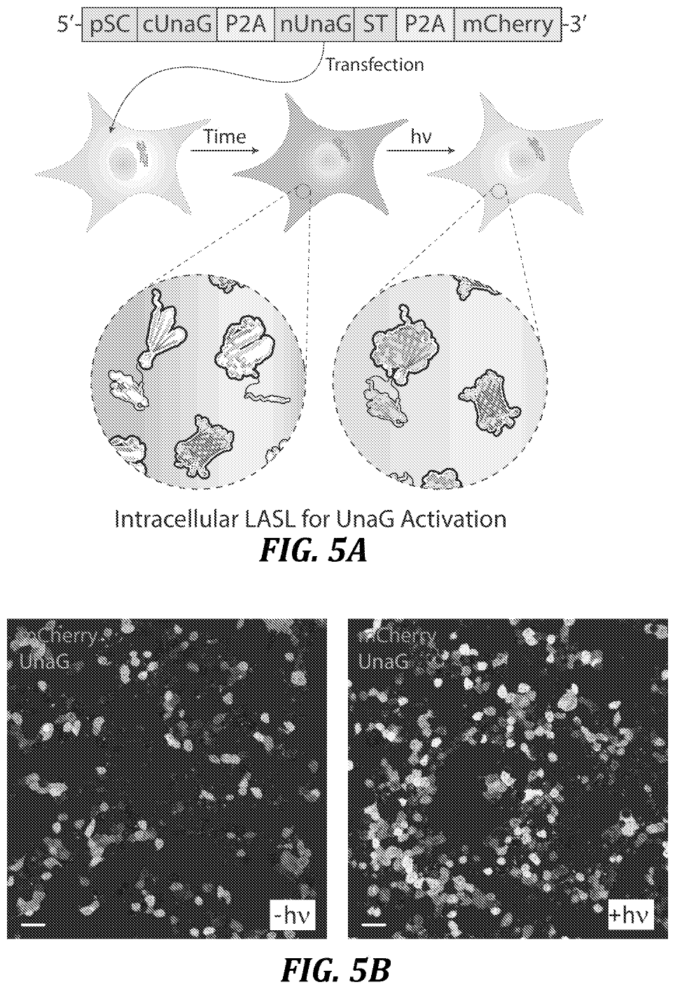

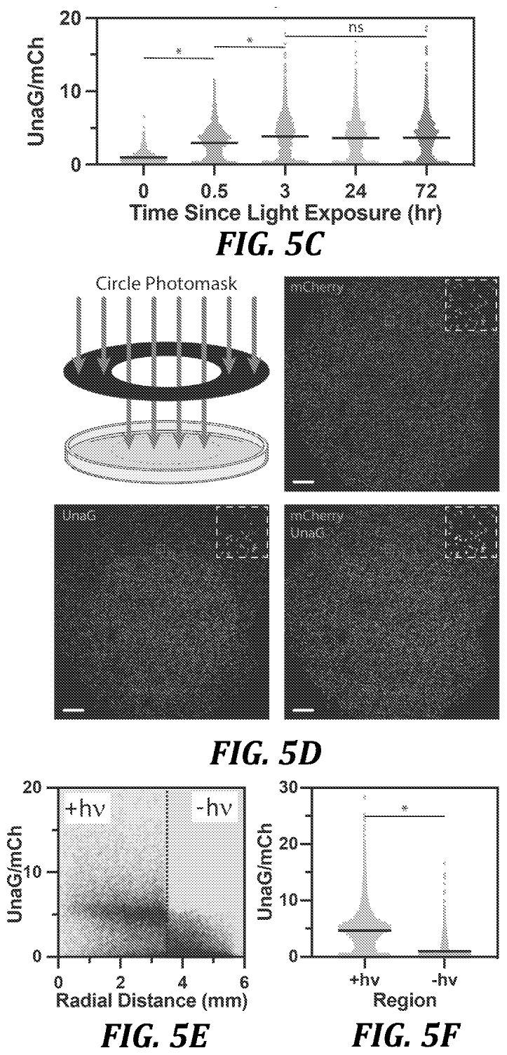

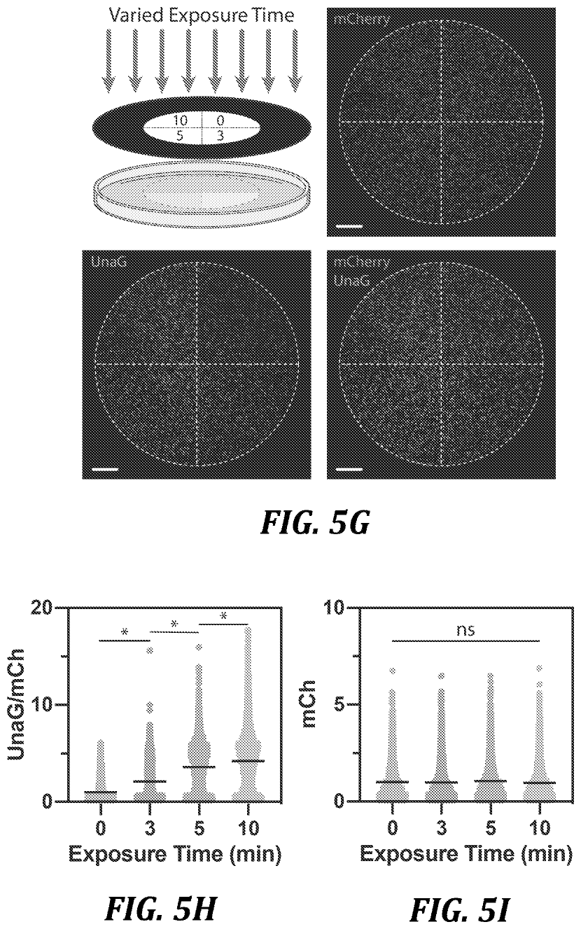

Having demonstrated that LASL could be used to functionally assemble protein fragments with predictable dose-dependence and well-defined kinetics in vitro using purified bacterial proteins, we pursued extension of the approach to irreversibly activate protein function spatiotemporally within living mammalian cells. Towards this goal, we cloned a polycistronic split-UnaG (SEQ ID No. 41) LASL construct consisting of three components—pSC-cUnaG (SEQ ID No. 39), nUnaG-ST (SEQ ID No. 31), and mCh—each separated by P2A (EXAMPLE 2 Method S25); this design was selected to ensure similar expression levels of each UnaG (SEQ ID No. 41) fragment and provide an internal standard (i.e., red fluorescence of mCh) to account for transfectional variations on a cell-by-cell basis (FIG. 5A). HEK-293T cells were co-transfected with plasmids encoding for the mutant MbPylRS/tRNA pair and split-UnaG (SEQ ID No. 41) components (EXAMPLE 2 Method S26). When grown in media supplemented with Lys(oNB), virtually all cells fluoresced red 24 h post-transfection, indicating high transfection efficiencies and amber codon read-through (FIG. 5B). As predicted, cells subjected to flood illumination (λ=365 nm, 20 mW cm z, 20 min) gained green fluorescence, reflecting successful LASL-mediated functional assembly of UnaG (SEQ ID No. 41) intracellularly, while unexposed cells did not (FIG. 5b). UnaG (SEQ ID No. 41)/mCh fluorescent ratios from individual cells were determined through automated image analysis and quantified over time; statistically significant increases (p<0.0001, unpaired t-test) for UnaG (SEQ ID No. 41)/mCh were observed for cells within 30 min of light exposure (=365 nm, 20 mW cm⁻², 7 min) with peak levels detected by 3 h while UnaG (SEQ ID No. 41)/mCh did not increase over 72 h for unexposed cells (FIG. 5C, FIG. 21). Though individual fluorophore signal decreased slightly over time (attributed to expected protein degradative clearance and/or dilution accompanying cell division), maximal UnaG (SEQ ID No. 41)/mCh fluorescence ratios (˜5-fold higher than unexposed samples) persisted in photostimulated cells for at least 72 h in a manner consistent with covalent UnaG (SEQ ID No. 41) assembly.

To further demonstrate LASL's ability to spatiotemporally and optically regulate specific biomacromolecular function in living systems, we focused our efforts towards patterning covalent protein assembly and concomitant activation within mammalian cell culture. Co-transfected HEK-293T cells were lithographically subjected to collimated light (λ=365 nm, 20 mW cm⁻², 20 min) through an open-circle photomask (diameter=7 mm); cells in the light-exposed region exhibited both green and red fluorescence, whereas those unexposed appeared primarily red (FIG. 5D). Quantification of individual cell UnaG (SEQ ID No. 41)/mCh fluorescence ratios as a function of radial distance from the mask's center revealed a clear activation pattern with micron-scale resolution (FIG. 5C). Aggregated analysis for all cells (˜35,000) demonstrated a ˜5-fold and statistically significant increase for UnaG (SEQ ID No. 41)/mCh in light-exposed regions relative to those in the unexposed outside ring (p<0.0001, unpaired t-tests) with no significant mCh photobleaching (FIG. 5F, FIG. 22). Exposing different regions of culture to light for variable amounts of time (0, 3, 5, 10 min), we observed the expected dose-dependent activation as indicated by significantly increasing UnaG (SEQ ID No. 41)/mCh fluorescent ratios (p<0.0001, unpaired t-tests) accompanying lengthened exposures (FIGS. 5G-5I).

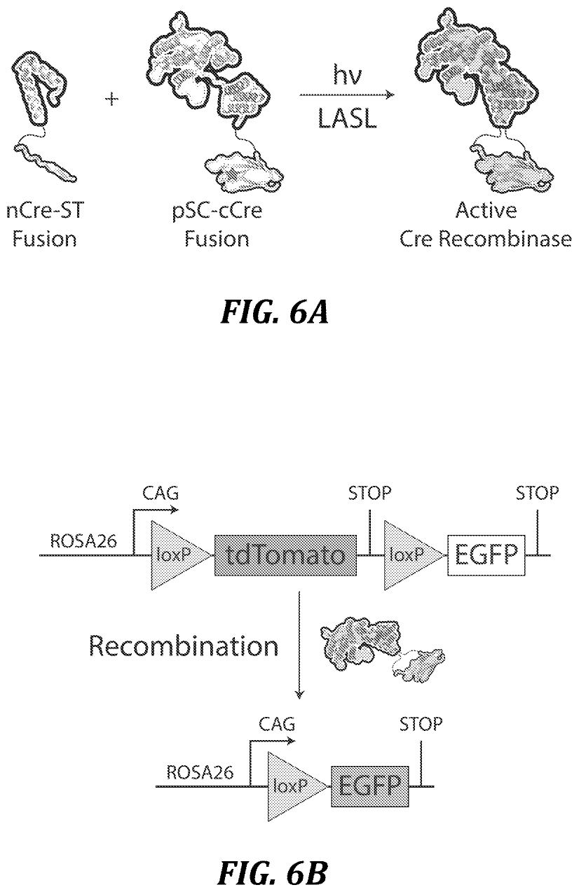

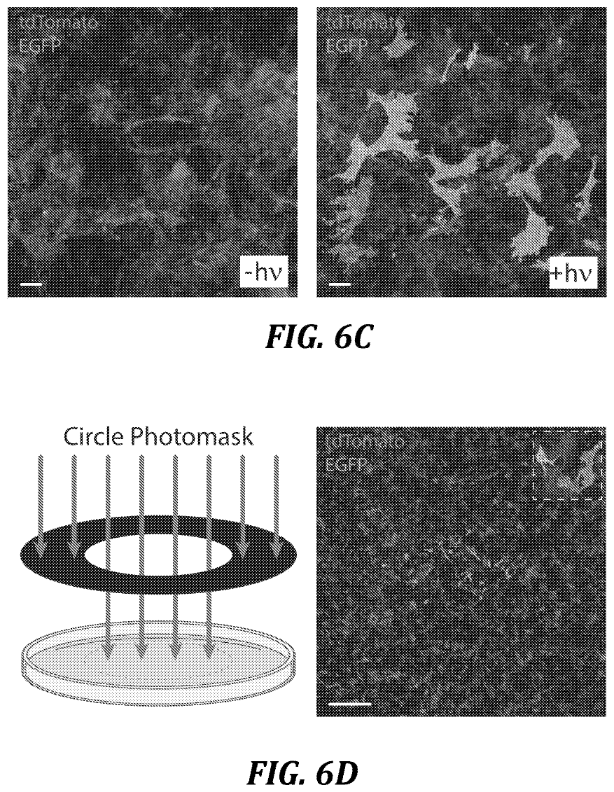

Following the successful generation of UnaG (SEQ ID No. 41) fluorescence intracellularly via LASL, we expanded our efforts to a protein target whereby its activation would yield lasting cellular functional changes, specifically in the form of irreversible genome editing. Towards this aim, we selected Cre recombinase, a topoisomerase that can recognize and site-specifically excise DNA between two loxP sites, which is commonly used for gene knock in/out studies in vivo. Exploiting a previously validated split site for rapamycin-induced Cre assembly (nCre=residues 19-59; cCre=residues 60-343), we created a polycistronic split-Cre LASL construct consisting of two components—nCre-ST and pSC-cCre—each separated by P2A and individually fused to nuclear localization sequences (FIG. 6A, EXAMPLE 2 Method 27). This cassette was transfected into transgenic mouse dermal fibroblasts bearing a Cre-dependent dual-color reporter in the “safe harbor” Rosa26 locus (EXAMPLE 2, Method S28); cells constitutiwely express red-fluorescent protein tdTomato but switch expression to EGFP upon Cre-mediated recombination (FIG. 6B). As designed, isolated primary cells fluoresced red but not green under normal culture conditions; upon light exposure (λ=365 nm, 20 mW cm⁻², 3 min), a significant percentage of cells stopped fluorescing red and turned green, reflecting success in optically restoring Cre activity via LASL and downstream genome editing (FIG. 6C). Directed illumination through a circle photomask (2 mm diameter opening) permitted spatial patterning of recombination and photoconditional gene regulation (FIG. 6D). All together, these experiments establish LASL's unique ability to irreversibly activate intracellular protein function with spatiotemporal control and in a dose-dependent manner.

### Example 2

### Method S1 Synthesis of Nε-(o-nitrobenzyloxycarbonyl)-L-lysine [Lys(oNB)]

### Synthesis of 2-nitrobenzyl-N-succinimidyl carbonate

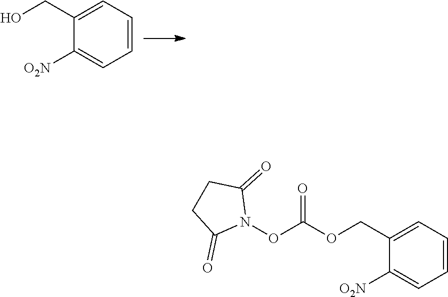 [SMILES: O=[N+]([O-])c1ccccc1CO ; O=C(OCc1ccccc1[N+](=O)[O-])ON1C(=O)CCC1=O]

2-nitrobenzyl-N-succinimidyl carbonate was synthesized as previously reported with minor modification. Disuccinimidyl carbonate (4.64 g, 18.1 mmol) was added to a mixture of 2-nitrobenzyl alcohol (2.5 g. 16.3 mmol) and triethylamine (3.4 mL, 24.5 mmol) in acetonitrile (80 mL) under nitrogen and stirred at room temperature for 30 min. The resulting reaction mixture was extracted with ethyl acetate (EtOAc, 3×) and washed with brine and water. The combined organic extracts were dried over Na₂SO₄ and concentrated under reduced pressure. Purification by flash column chromatography (Hexanes:EtOAc, 1:1) gave 2-nitrobenzyl-N-succinimidyl carbonate as a straw-colored oil (3.11 g, 65%).

### Synthesis of Nε-(o-nitrobenzyloxycarbonyl)-L-lysine [Lys(oNB)]

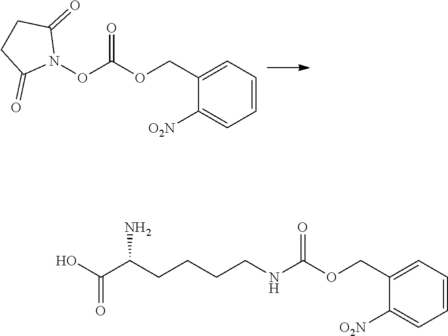 [SMILES: O=C(OCc1ccccc1[N+](=O)[O-])ON1C(=O)CCC1=O ; N[C@H](CCCCNC(=O)OCc1ccccc1[N+](=O)[O-])C(=O)O]

To a solution of 2-nitrobenzyl-N-succinimidyl carbonate (3.11 g, 10.6 mmol) in dimethylfuran (50 mL) was added N_{α}-Boc-L-lysine (2.87 g, 11.7 mmol, Chem-Impex) and N,N-diisopropylethylamine (7.35 mL, 42.4 mmol) and stirred overnight at room temperature. The resulting reaction mixture was extracted into EtOAc (3×) and washed with brine and water. The combined organic extracts were concentrated under reduced pressure prior to purification by flash column chromatography (Hexanes:[EtOAc with 1% acetic acid], 1:1) to give Boc-protected Lys(oNB) as a light brown oil (3.08 g, 69%). The Boc-protected Lys(oNB) was dissolved in 3 mL 1,4-dioxane and 30 mL of 4 N HCl in 1,4-dioxane was added dropwise. The reaction was stirred at room temperature for 2.5 h. concentrated under reduced pressure, then subsequently triturated with 3×15 mL of diethyl ether to give the pure product (1.78 g, 51.4%, [Nε-(o-nitrobenzyloxycarbonyl)-L-lysine, denoted Lys(oNB)]) as a white solid. ¹H NMR (300 MHz, DMSO d6): S=1.28-1.33 (m, 1H), 1.42 (s, 3H), 1.67-1.81 (m, 2H), 2.99 (d, J=6.0 Hz, 2H), 5.35 (s, 2H), 7.42 (t, J=7.7 Hz. 1H), 7.58-7.65 (m, 2H), 7.80 (td, J=7.7 Hz, 1H), 8.11 (dd, J=7.9 Hz, 1H). MS (LTQ-MS): calculated for C₁₄H₂₀N₃O₆ ([M+H]⁺): 326.1; found, 326.1. These spectral data matched those previously reported.

Method S2 Construction of aaRS/tRNA Plasmids for Lys(oNB) Incorporation

### Bacterial Incorporation of Lys(oNB)

The exogenous translational machinery used here was based on previously published work. The plasmids pEVOL-MmPylRS and pBK-oNBK-1 were generously gifted by Peter Schultz. The pEVOL expression vector contains a single copy of Pyl-tRNA_{CUA} under the proK promoter, two copies of Methanosarcina maize pyrrolysyl-tRNA synthetase (MmPylRS) under araBAD and ginS' promoters, and CmR for chloramphenicol resistance. The evolved synthetase NBK-1 contains mutations Y306M, L309A, C348A, Y384F. These plasmids were used to generate the appropriate fragments for Gibson Assembly to create pEVOL-NBK-1.

NBK-1 was amplified through Polymerase Chain Reaction (PCR) from the cloning plasmid pBK-oNBK-1 with primers NBK-1-a Forward and -Reverse (SEQ ID No. 15, SEQ ID No. 16, EXAMPLE 2, Table 2). These primers provide N-terminal homology with the ribosomal binding site of pEVOL and C-terminal homology with the N-terminus of a gene fragment, rrnB-glnS(SEQ ID No. 48, Integrated DNA Technologies, see “DNA sequences for all cloned constructs”), which contained the appropriate machinery needed between the two copies of NBK-1. A second PCR reaction was performed on pBK-oNBK-1 with primers NBK-1-b Forward and -Reverse (SEQ ID No. 17 SEQ ID No. 18, EXAMPLE 2 Table 2). These primers provide N-terminal homology with the C-terminus of rrnBl-glnS (SEQ ID No. 48) and C-terminal homology with pEVOL. The vector backbone was amplified by PCR with primers pEVOL-Forward and -Reverse (SEQ ID No. 19, SEQ ID No. 20, EXAMPLE 2 Table 2Error! Reference source not found.). PCR products were purified by agarose gel electrophoresis, excised, and extracted by QlAprep column (Qiagen). The final pEVOL-NBK-1 plasmid was constructed by Gibson Assembly. The vector backbone was mixed with rrnB-glnS (SEQ ID No. 48) and two copies of NBK-1 each at a 3-fold molar excess relative to the backbone. This mixture was then diluted 1:1 (v/v) with 2× Gibson Assembly Master Mix (New England BioLabs Inc.) and reacted at 50° C. for 1 h before transformation into chemically competent Top10 K coil. Proper assembly was confirmed by Sanger Sequencing with primers glnS-Seq (SEQ ID No. 21) and pEVOL-REV-Seq (SEQ ID No. 22).

### Mammalian Incorporation of Lys(oNB)

For efficient incorporation of Lys(oNB) in mammalian cells, a plasmid containing a mutant Methanosarcina bakeri synthetase (MbPylRS; Y271A, Y349F) and four copies of an engineered M15-tRNA_{CUA} recently reported to boost incorporation of non-canonical amino acids in pyrrolysine-based systems was used.

### Method S3 Molecular Cloning of Plasmids for Expression of pSC (SEQ ID No. 26) Variants

### SC

The SC (SEQ ID No. 25) gene (Addgene, Plasmid #35044, pDEST14-SpyCatcher) was amplified through PCR using primers that included 5′ NdeI and 3′ XhoI restriction enzyme digestion sites (SpyCatcher-Forward (SEQ ID No. 1) and -Reverse (SEQ ID No. 2), EXAMPLE 2 Table 2). The amplified gene and pET21 expression plasmid (Novagen) were double digested with FastDigest™ NdeI and XhoI (Thermo Scientific) for 1 h at 37° C., separated using agarose gel electrophoresis (1% UltraPure Agarose, Thermo Scientific), and extracted with the QIAquick Gel Extraction Kit (Qiagen). SC was ligated into the pET21 expression plasmid (overnight at 16° C., T4 DNA ligase—New England BioLabs Inc.). Chemically competent Top10 E. coli (Thermo Scientific) were transformed with the ligation product through heat shock (42° C., 30 s), incubated in SOC media (1 h at 37° C.; 20 g L⁻¹ tryptone, 5 g L⁻¹ yeast extract, 0.5 g L⁻¹ NaCl, 2.5 mM KCl, 10 mM MgCl₂, 10 mM MgSO₄, 20 mM glucose) and spread on agar plates (10 g L⁻¹ tryptone, 5 g L⁻¹ yeast extract, 10 g L⁻¹ NaCl, 15 g L⁻¹ agar) containing carbenicillin (100 ug mL⁻¹) overnight. A single colony was grown overnight in 5 mL of Miller's LB (10 g L⁻¹ tinptone, 5 g L⁻¹ yeast extract, 10 g L⁻¹ NaCl) and plasmid DNA was collected using the QIAprep Spin Minprep Kit (Qiagen). DNA sequences were confirmed through Sanger Sequencing (GeneWiz).

### pSC (SEQ ID No. 26)

The photocaged SpyCatcher (SEQ ID No. 26, pSC) plasmid was constructed through site-directed mutagenesis of the wild-type SC plasmid at Lys31. The SC plasmid generated above was mutated to the photocaged variant by PCR amplification using overlapping forward and reverse primers each containing the amber stop codon TAG in-place of Lys31 (SpyCatcher-SDM Forward (SEQ ID No. 3)- and -Reverse (SEQ ID No. 4), EXAMPLE 2 Table 2). Remaining SC plasmid was digested with DpnI (37° C., 2 h; New England BioLabs Inc.) and heat inactivated (80° C., 20 min) before transformation into chemically competent Top10 E. coli. The mutation was confirmed through Sanger Sequencing (GeneWiz).

pSC-LPETG (SEQ ID No. 27)

pSC-LPETG (SEQ ID No. 27) variant was generated by PCR of the SC plasmid using primers that included 5′ NdeI digestion site and the 3′ addition of -LPETG- and XhoI digestion site (pSC-LPETG (SEQ ID No. 27) Forward and -Reverse (SEQ ID No. 5, SEQ ID No. 6), EXAMPLE 2 Table 2). The PCR product and pET21 expression vector were digested with NdeI and XhoI (New England BioLabs Inc.) overnight at 37′C. Digests were ligated (overnight at 16° C., T4 DNA ligase—New England BioLabs Inc.) to form the desired expression vector as confirmed by Sanger Sequencing. The photocaged variant (Lys31TAG) was generated through site-directed mutagenesis as described above and confirmed by Sanger Sequencing.

### Method S4 Molecular Cloning of SpyTag (SEQ ID No. 7, SEQ ID No. 8) Fusion Constructs

### GST-ST (SEQ ID No. 28)

A glutathione-S-transferase-ST fusion (GST-ST (SEQ ID No. 28)) was received as used by ordering 5×(GGS)-SpyTag inserted at the BamHI and XhoI sites of the plasmid pGEX-4T-1 (GenScript).

mRuby-ST

An mRuby-ST (SEQ ID No. 29) fusion was generated by double digesting a pET21 expression vector containing a 5′ mRuby with a 3′ HindIII and XhoI multiple cloning site with HindIII and XhoI restriction enzymes (overnight at 37° C., New England BioLabs Inc.). The digest was purified as described in Method S3. Single-stranded forward and reverse DNA oligos encoding SpyTag (SEQ ID No. 7, SEQ ID No. 8) with a 5′ HindIII and 3′ XhoI restriction site (Integrated DNA Technologies) were designed such that oligos would anneal to generate sticky ends. Equimolar amounts of each oligo were heated in annealing buffer (10 mM tris, pH 7.5, 50 mM NaCl, 1 mM EDTA) in a heat block (95° C.) for 5 min, after which the block was turned off, allowing the oligos to anneal as they cooled to room temperature (1 h). The annealed oligos were used as is for ligation into the digested pET21-mRuby vector which was subsequently transformed into chemically competent Top10 E. coli. The fusion was confirmed with Sanger Sequencing (GeneWiz).

### Method S5 Protein Expression and Purification

### General Protein Expression and Purification

BL21(DE3) E. coli (Promega) were transformed with the appropriate plasmid for the desired expression. Small cultures were grown overnight in Miller's lysogeny broth (LB) (10 g L⁻¹ tryptone. 5 g L⁻¹ yeast extract. 10 g L⁻¹ NaCl) with the appropriate antibiotic (carbenicillin at 100 μg mL⁻¹ or kanamycin at 50 μg mL⁻¹). Large cultures (0.25-1 L, Miller's LB supplemented with antibiotic) were inoculated at a 1:50 ratio and incubated with agitation (250 rpm) at 37° C. until an optical density at λ=600 nm of 0.6. Protein production was induced with the addition of isopropyl O-D-1-thiogalactopyranoside (IPTG, final concentration of 0.5 mM). Cultures were agitated overnight at 16° C., then collected through centrifugation (4,000×g for 10 min, 4° C.). Cell pellets were resuspended in lysis buffer (40 mL, 20 mM Tris, 50 mM NaCl, 10 mM imidazole) and sonicated (0° C., 6×3 min cycles at 30% amplitude. 33% duty cycle with 3 min rest). Cell lysate was clarified by centrifugation (5,000×g, 20 min) before purification via immobilized metal affinity chromatography (IMAC) using Ni-NTA resin (2 mL slurry, Gold Biotechnology). After discarding the flow-through, the resin was washed (6 mL, 20 mM Tris, 50 mM NaCl, 20 mM imidazole, pH 7.5) until minimal protein was observed in the flowthrough (λ_{abs}=280 nm) and protein was eluted (20 mM Tris, 50 mM NaCl, 250 mM imidazole, pH 7.5). Imidazole was removed through dialysis (ThermoFisher SnakeSkin MWCO 10 or 30 kDa Dialysis Tubing was selected based on protein size) against Tris buffer (20 mM Tris, 50 mM NaCl, pH 7.5) at 4° C. and spin concentrated (Amicon® Ultra-15; MWCO 3.5, 10, or 30 kDa was selected based on protein size). Proteins were stored at −20° C. in Tris buffer with 8% glycerol.

### pSC Expression and Purification

Expression and purification of pSC (SEQ ID No. 26) followed the above protocol except with the following modifications. BL21(DE3) E. coli (Promega) were doubly transformed with equimolar amounts of the expression vector and the corresponding amber suppression machinery (pEVOL-NBK-1, Method S2). Cells were grown overnight (37° C. agitated at 250 rev min⁻¹) in Terrific Broth (TB; 24 g L⁻¹ yeast extract. 20 g L⁻¹ tryptone, 4 mL L⁻¹ glycerol, 17 mM KH₂PO₄, 72 mM K₂HPO₄) supplemented with carbenicillin (100 μg mL⁻¹) and chloramphenicol (25 μg mL⁻¹). Large cultures (125 mL, TB) supplemented with carbenicillin (100 μg mL⁻¹) and chloramphenicol (25 μg mL⁻¹) were inoculated at a 1:25 and grown until reaching an optical density of 0.6 (λ=600 nm). Lys(oNB) dissolved in 2 equivalents 0.5 M NaOH was added to a final concentration of 1 mM and cultures were protected from ambient light. Simultaneously, cultures were induced by the addition of arabinose (final amount of 0.1%) and IPTG (final concentration of 0.5 mM). Cultures were agitated overnight at 16° C. then pelleted by centrifugation at 4,000×g for 10 min at 4° C. Cell pellets were resuspended in lysis buffer (40 mL: 20 mM Tris, 50 mM NaCl, 10 mM imidazole, pH 7.5) supplemented with EDTA-free protease inhibitor cocktail (1 large tablet, Pierce). Protein purification, dialysis, and spin concentration were performed as described above. pSC (SEQ ID No. 26) expression yielded approximately 5-10 mg pure protein per L of culture. pSC (SEQ ID No. 26) was stored frozen (−20° C.) in Tris buffer with 8% glycerol until needed.

### GST-ST (SEQ ID No. 28) Purification

GST-ST (SEQ ID No. 28) was expressed in a 500 mL culture (LB supplemented with carbenicillin at 100 μg mL⁻¹) as described above with the following modifications. Pelleted cells were resuspended in GST Equilibration Buffer (40 mL; 50 mM Tris, 150 mM NaCl, pH 8.0). Cells were lysed via sonication and debris was cleared through centrifugation as described above. Pierce Glutathione Agarose resin (1 mL slurry, Thermo Scientific) was prewashed with GST Equilibration Buffer (5 mL, 2×) before the soluble fraction was added. The protein-resin solution was rocked at 4° C. for 1 h to allow adequate binding. The solution was centrifuged (500×g, 2 min) and the supernatant decanted. The resin was then washed in batch with 5 mL (GST Equilibration Buffer) followed by centrifugation (500×g, 2 min) and decanting until absorbance at 280 nm reached baseline. GST-ST (SEQ ID No. 28) was collected by washing the resin with elution buffer (2 mL, 50 mM Tris, 150 mM NaCl, 10 mM reduced glutathione, pH 8.0) and then centrifuging (500×g, 2 min) to pellet the resin. The purified protein was decanted and applied to a Zeba Spin Desalting Column (7k MWCO, 10 mL, Thermo Scientific) equilibrated with GST Equilibration Buffer to remove reduced glutathione. GST-ST (SEQ ID No. 28) expression yielded approximately 100 mg L⁻¹ of culture and was stored at −20° C. in GST Equilibration Buffer with 8% glycerol.

### Method S6 Mass Spectrometric Analysis of Purified Proteins

Protein identity and purity was confirmed by liquid chromatography-mass spectrometry (LC-MS). Each protein species (1 μg in buffer containing 20 mM Tris, 50 mM NaCl, pH 7.4) was injected into an LC-MS (AB Sciex 5600 QTOF) using an inline polymeric reversed-phase column (PL 1912-1503, Agilent). Protein solutions were separated over an 8-min linear gradient from 5-95% acetonitrile in water with 0.1% formic acid at 0.5 mL min⁻¹. Mass spectrum scans were taken every 1 sec in positive mode. The chromatogram was integrated, and the full molecular weight was calculated using Analyst (AB Sciex). Observed and expected masses of all modified proteins are given in Table 1.

TABLE 1
Protein validation through mass spectrometry

| | Expected | Observed |
|---|---|---|
| | Mass | Mass |
| Protein Identity | (Da) | (Da) |
| SC (SEQ ID No. 25) | 13,209 | 13,209 |
| pSC (SEQ ID No. 26) | 13,388 | 13,388 |
| pSC-(SEQ ID No. 26)N₃ | 13,462 | 13,460 |
| GST-ST (SEQ ID No. 28) | 28,771 | 29,687 |
| SrtA_{7M}(SEQ ID No. 30) | 17,851 | 17,850 |
| mRuby-ST (SEQ ID No. 29) | 29,321 | 29,321 |
| nUnaG-ST (SEQ ID No. 31) | 53,314 | 53,314 |
| cUnaG-ST (SEQ ID No. 33) | 50,304 | 50,305 |
| SC-nUnaG (SEQ ID No. 35) | 23,322 | 23,323 |
| nUnaG-SC (SEQ ID No. 36) | 23,665 | 23,665 |
| SC-cUnaG (SEQ ID No. 37) | 20,293 | 20,293 |
| cUnaG-SC (SEQ ID No. 38) | 20,655 | 20,655 |
| pSC-cUnaG (SEQ ID No. 39) | 20,472 | 20,472 |
| pSC-cUnaG-LPETG (SEQ ID No. 40) | 20,700 | 20,700 |
| pSC-cUnaG (SEQ ID No. 39)-N₃ | 20,144 | 20,144 |
| pSC-SmBiT (SEQ ID No. 42) | 15,818 | 15,818 |
| LgBiT-ST (SEQ ID No. 43) | 20,210 | 20,210 |

Method S7 Assessing LASL Light Dependency with Purified Proteins

Purified pSC (SEQ ID No. 26, 20 μM) was treated with varied dosages of light (λ=365 nm, 10 mW cm⁻², 0-90 min). GST-ST (SEQ ID No. 28, 25 μM) was added and reacted (18 h, 37° C.). Samples were diluted 1:1 with 2× Laemmli sample buffer and briefly boiled before analysis by SDS-PAGE (8-16% TGX precast mini-gel, BioRad). Experimental replicates (n=4) were quantified by determining the band intensity of the two reactants (pSC (SEQ ID No. 26) and GST-ST (SEQ ID No. 28)) and the LASL product at each time point in FIJI. Photokinetics were determined by normalizing each replicate by their asymptotic value found by a first-order fit. Normalized data were averaged and fit to determine the rate constant and half-life (GraphPad Prism).

Method S8 Demonstrating LASL Compatibility with Bacterial Cell Lysate

To generate bacterial cell lysate, unmodified Top10 E. coli(Promega) were cultured overnight with shaking (37° C., 200 RPM) in Miller's LB (125 mL, 10 g L⁻¹ tryptone, 5 g L⁻¹ yeast extract, 10 g L⁻¹ NaCl). Cells were pelleted by centrifugation (4000×g, 10 min, 4° C.), resuspended in Tris buffer (40 mL, 20 mM Tris, 50 mM NaCl), and lysed by sonication (Method S5). Cell debris was cleared by centrifugation (5000×g, 20 min, 4° C.) and the soluble fraction was supplemented with LASL components to test reaction in complex biological solutions. Purified pSC (SEQ ID No. 26) was exposed to light (λ=365, 45 min, 20 mW cm⁻²). SC (SEQ ID No. 25), pSC (SEQ ID No. 26), and light-treated pSC (SEQ ID No. 26, 10 μM) were added individually with GST-ST (SEQ ID No. 28, 10 μM) to cell lysate (10 μL, 20 μL reaction volume) and allowed to react (30 min, 37° C.). Samples were diluted 1:1 with 2× Laemmli sample buffer (BioRad) and boiled briefly before analysis by SDS-PAGE (8-16% TGX precast mini-gel. BioRad).

### Method S9 Performing LASL in Mammalian Cell Lysate

To generate mammalian cell lysate, HEK-293T cells were cultured in a 25 cm² flask until confluent. Cells were scraped into ice-cold PBS (5 mL), pelleted by centrifugation (200×g, 5 min, 4° C.), washed with ice-cold PBS (5 mL), and pelleted by centrifugation (200×g, 5 min, 4° C.). The cell pellet was resuspended in M-PER Mammalian Protein Extraction Reagent (Thermo Scientific, 3 mL), and vortexed (3× for 10 min over 45 min). Cell debris was cleared by centrifugation (14000×g, 20 min. 4° C.) and the soluble fraction was supplemented with LASL components to test reaction in complex biological solutions. Equal molar amounts of purified SC (SEQ ID No. 25) and pSC (SEQ ID No. 26) were combined with GST-ST (SEQ ID No. 28, 10 μM) and added to cell lysate (10 μL, 20 μL reaction volume). Solutions were exposed to light (λ=365 nm, 20 mW cm⁻², 45 min) and allowed to react (30 min, 37° C.). Samples were diluted 1:1 with 2× Laemmli sample buffer (BioRad) and boiled briefly before analysis by SDS-PAGE (8-16% TGX precast mini-gel, BioRad).

Method S10 Chemoenzymatic Synthesis of the Azide-Tagged pSC (SEQ ID No. 26, pSC-N₃) Via Sortagging

Staphylococcus aureus sortase A heptamutant (SrtA_{7M} (SEQ ID No. 30): P94R, D160N, D165A, Y187L, E189R, K190E, K196T) was added to pSC-LPETG (SEQ ID No. 27) in Tris Buffer (20 mM Tris, 50 mM NaCl, pH 7.5) at a 1:10 molar ratio and supplemented with a 10-fold molar excess of H-SEQ ID No. 50(N₃)—NH₂ peptide. After reaction (2 h, 37° C.), SrtA_{7M} (SEQ ID No. 30) and unreacted pSC-LPETG (SEQ ID No. 27) were removed through reverse IMAC purification with the addition of Ni-NTA agarose resin (Gold Biotechnology). The column flow-through containing the azide-functionalized product was buffer exchanged (MWCO˜7000 Da, Zeba Spin Desalting Column, Thermo Scientific) against PBS (pH=7.4) to remove excess peptide and generate pSC-N₃ (SEQ ID No. 26). Protein identity and azide-functionalization were confirmed by mass spectrometry (Table S1).

Method S11 Spatiotemporally Controlled Protein Immobilization within Gels Via LASL

A solution of PEG-tetraBCN (Mₙ˜20,000 Da, 4 mM) and pSC-N₃ (SEQ ID No. 26, 14 μM) were pre-reacted for 2 h at room temperature in PBS. PEG-diazide (Mₙ˜3,500 Da, 8 mM) crosslinker was added and the gel-precursor solution was aliquoted between Rain-X®-treated glass slides with silicone rubber spacers (0.5 mm thick, McMaster-Carr). Network formation was allowed to proceed for 1 h at room temperature before incubating gels in excess PBS overnight.

For gels patterned photolithographically, a chrome photomask of tessellated gecko silhouettes (Photo Sciences) was applied between the gel and a Lumen Dynamics OmniCure S1500 Spot UV curing system equipped with an internal band-pass filter (λ=365 nm) and a second in-line cut-on long-pass filter during exposure (λ=365 nm, 20 mW cm⁻², 15 min). To pattern gradients, a programmable linear motion stage was moved across rectangular, 6 mm long gels at various rates (0.133, 0.2, 0.4 mm min⁻¹) during exposure to light (λ=365 nm, 15 mW cm⁻²). Gels were incubated in a solution of mRuby-ST (SEQ ID No. 29, 6.8 mM, PBS) overnight at room temperature. Excess mRuby-ST (SEQ ID No. 29) was removed by incubating in PBS with 1% penicillin/streptomycin (PYS) (overnight, 37° C.) before fluorescence imaging on a Stellaris 5 confocal microscope equipped with a 10× dry objective (Leica).

### Method S12 Design of Polycistronic LASL Constructs to Optically Specify Subcellular Protein Localization

SC-mCh-P2A-ST-EGFP-CAAX and EGFP-ST-P2A-SC-CAAX cassettes were purchased and received cloned in the pcDNA3.1 vector (GenScript). The plasmids were individually transformed into electrocompetent Top10 E. coli (ThermoFisher). Overnight cultures (10 mL Miller's LB, 100 μg mL¹ carbenicillin) were pelleted at 4000×g for 10 min and plasmid DNA was collected using the QIAprep Spin Miniprep kit (Qiagen). Plasmid DNA was eluted in cell culture grade dH₂O (Corning) and used for mammalian cell transfections.

Site-directed mutagenesis was performed to generate pSC-mCh-P2A-ST-EGFP-CAAX (SEQ ID No. 47) and EGFP-ST-P2A-pSC-CAAX (SEQ ID No. 46) from the wild-type plasmid (Method S3).

### Method S13 Mammalian Cell Culture, Transfection, and Optical Membrane Tagging by LASL

### General Methods

HEK-293T cells were maintained at 37° C. and 5% CO₂ in DMEM supplemented with 10% fetal bovine serum (FBS) and 1% P/S. Cells were seeded at 50,000 cells cm-2 on 35 mm glass bottom dishes (14 mm glass microwell size. Cellvis) coated with 0.1% gelatin 24 h prior to transfection. Upon transfection, cells were swapped to complete DMEM containing Lys(ONB) (2.5 mM). Equal amounts (0.5 μg) of pNeu-hMbPylRS-4×U6M15 and either the pSC-mCherry-GFP-ST-CAAX or GFP-ST-pSC-CAAX expression vector were co-transfected using Lipofectamine 3000 following the supplied protocol.

### Light Treatment

24 h post-transfection, media was swapped with Hank's Balanced Salt Solution containing calcium and magnesium (HBSS, Gibco) but not Lys(oNB) prior to treatment with light (λ=365 nm, 20 mW cm⁻², 0-20 min). Cells were incubated (6 h for GFP-ST-pSC-CAAX, 12 h for pSC-mCherry-GFP-ST-CAAX) in complete DMEM without Lys(oNB) before imaging. Cells were swapped to HBSS and imaged on a Stellaris 5 confocal microscope (Leica) equipped with a 40× objective immersed in oil (λ_{EGFP excition}=489 nm, λ_{mCherry excitation}=587 nm).

### CellProfiler Analysis

Individual cell fluorescence and position was quantified using CellProfiler¹⁰. Cells were first identified through mCh or EGFP fluorescence via the IdentifyPrimaryObjects module. In-cell fluorescence was determined via the MeasureObjectIntensityDistribution module with 21 internal bins/cell. Square heatmap histograms were generated in GraphPad Prism prior to conversion into a circular form using Adobe Illustrator.

### Method S14 Molecular Cloning of Split-UnaG (SEQ ID No. 41) Fragments for SpyLigation and LASL

UnaG (SEQ ID No. 41) was genetically split between residues Lys84 and Gly85 to form an N-terminal domain, nUnaG (residues 1-84), and a C-terminal domain, cUnaG (residues 84-139). The UnaG-ST (i.e., nUnaG-ST (SEQ ID No. 31), ST-nUnaG (SEQ ID No. 32), cUnaG-ST, ST-cUnaG (SEQ ID No. 34)) and —SC (i.e., SC-nUnaG (SEQ ID No. 35), nUnaG-SC (SEQ ID No. 36), SC-cUnaG (SEQ ID No. 37), cUnaG-SC (SEQ ID No. 38)) variants were ordered cloned into pET29a(+) and pET21a(+) vectors, respectively, at the NdeI and XhoI cloning sites (GenScript).

After difficulty expressing the ST-nUnaG (SEQ ID No. 32) and ST-cUnaG (SEQ ID No. 34) variants, the solubilizing maltose binding protein (MBP) was fused to the N-termini of each by Gibson assembly. MBP was amplified by PCR using primers N-MBP-SpyTag Forward and -Reverse (SEQ ID No. 9, SEQ ID No. 10, EXAMPLE 2 Table 2), purified via agarose gel electrophoresis, excised, and further purified with a QIAprep miniprep column (Qiagen). The ST-nUnaG (SEQ ID No. 32) and ST-cUnaG (SEQ ID No. 34) vectors were amplified by PCR using primers SpyTag-UnaG Forward and -Reverse (SEQ ID No. 13, SEQ ID No. 14, EXAMPLE 2 Table 2) and purified. The amplified vector and insert were mixed at a 1:3 molar ratio, diluted 1:1 with 2× Gibson Assembly Master Mix (New England BioLabs Inc.), and incubated at 50° C. for 1 h. Chemically competent Top10 E. coli were transformed, and insertion was confirmed with Sanger Sequencing (GeneWiz). The addition of MBP to the N-termini did not resolve expression issues and these variants were not investigated further.

Upon initial difficulty expressing the nUnaG-ST (SEQ ID No. 31) and cUnaG-ST (SEQ ID No. 33) variants, MBP was cloned C-terminal to ST using restriction cloning.

Here. MBP was amplified through PCR using primers C-SpyTag-MBP Forward and -Reverse (SEQ ID No. 11, SEQ ID No. 12), which include N-terminal HindIII and C-terminal XhoI digestion sites. The purified product and nUnaG-ST (SEQ ID No. 31) and cUnaG-ST (SEQ ID No. 33) vectors were digested with HindIII-HF and XhoI (New England BioLabs Inc.) for 4 h at 37° C. prior to heat inactivation at 80° C. for 20 min. The digests were ligated at an equimolar ratio and transformed into chemically competent Top10 E. coli. Fusions were confirmed with Sanger Sequencing (GeneWiz). Following fusion with MBP, soluble expression of pure protein was obtained.

Site-directed mutagenesis was performed to generate pSC-cUnaG (SEQ ID No. 39) from the SC-cUnaG (SEQ ID No. 37) plasmid (Method S3).

Protein identities were confirmed by mass spectrometry (Table 1).

### Method S15 Fluorescence Reconstitution of Purified UnaG (SEQ ID No. 41) Variants Through SpyLigation

Protein concentrations were estimated for the UnaG-SpyCatcher variants (nUnaG-SC (SEQ ID No. 36), cUnaG-SC (SEQ ID No. 38). SC-nUnaG (SEQ ID No. 35), SC-cUnaG (SEQ ID No. 37)) and UnaG-SpyTag variants (SEQ ID No. 32) that were readily soluble during expression and purification (nUnaG-ST (SEQ ID No. 31), cUnaG-ST) by absorbance (λ=280 nm). Individual proteins and appropriate binding partners (i.e., nUnaG-cUnaG and SC (SEQ ID No. 25)-ST (SEQ ID No. 7, SEQ ID No. 8) pairing) were mixed (10 μM, Tris Buffer) with the substrate bilirubin (20 μM final concentration). Fluorescence was measured 0.5 and 24 h after mixing (37° C., in the dark) with a fluorescent plate reader in 96-well, black opaque plates (λ_{excitation}=480 nm, λₑₘᵢₛₛᵢₒₙ=575 nm).

### Method S16 Expression and Purification of Wild-Type UnaG (SEQ ID No. 41)

The wild-type UnaG (SEQ ID No. 41) protein (Addgene, Plasmid #163125, pMAL-c5x_UnaG (SEQ ID No. 41)) was expressed in NEB-Express E. coli (New England BioLabs Inc.) and purified using IMAC as described for other proteins (Method S5). Protein concentration was estimated by absorbance (λ=280 nm) prior to fluorescence measurements.

### Method S17 Light-Dependent Fluorescence Reconstitution of Split-UnaG (SEQ ID No. 41) Variants by LASL

Fluorescence reconstitution relative to concentration was determined by mixing nUnaG-ST (SEQ ID No. 31) with light-treated (30 min, λ=365 nm at 20 mW cm⁻²) or untreated pSC-cUnaG (SEQ ID No. 39). Species were mixed at equimolar concentrations (0-10 μM) in Tris Buffer supplemented with bilirubin (7 μM). Fluorescence was measured 0.5 and 24 h after the reaction began using a fluorescent plate reader (λ_{excitation}=495 nm, λₑₘᵢₛₛᵢₒₙ=528 nm). Similarly, solutions of pSC-cUnaG (SEQ ID No. 39, 14 μM in Tris Buffer) were exposed to UV light (λ=365 nm, 20 mW cm⁻², 0-30 min). After light exposure, equal molar amounts of each light exposed pSC-nUnaG were combined with nUnaG-ST (SEQ ID No. 31, 7 μM) and bilirubin (7 μM) and incubated (24 h at 37° C.). Fluorescence measurements were obtained on a fluorescent plate reader (λ_{excitation}=485 nm, λₑₘᵢₛₛᵢₒₙ=528 nm).

### Method S18 Determination of Excitation/Emission Spectra of Reconstituted UnaG (SEQ ID No. 41)

The fluorescence excitation and emission spectra of reconstituted UnaG (SEQ ID No. 41) was measured by mixing nUnaG-ST (SEQ ID No. 31) and SC-cUnaG (SEQ ID No. 37), pSC-cUnaG (SEQ ID No. 39), or pSC-cUnaG (SEQ ID No. 39) treated with light (λ=365 nm, 20 mW cm⁻², 20 min; individual species at 1 μM). Ligation was allowed to proceed overnight prior to fluorescence spectral determination by scanning emission. λ_{max,excitation}=491 nm, λ_{max,emission}=528 nm.

### Method S19 Synthesis of SpyTag-Azide Peptide (SEQ ID No. 7, SEQ ID No. 8, ST-N3)

The resin-bound peptide Boc—SEQ ID No. 51 (Dde)-NH₂ (where the SpyTag (SEQ ID No. 7, SEQ ID No. 8) sequence is underlined) was synthesized by microwave-assisted Fmoc solid-phase peptide synthesis (CEM Liberty 1, 0.5 mmol scale) on Rink amide resin (0.5 mmol scale). Fmoc deprotections were performed in 20% piperidine (v/v) in dimethylformamide (DMF) with 0.1 M 1-hydroxybenzotriazole (HOBt) at 90° C. for 90 sec. Amino acids containing standardly protected side chains were coupled to resin-bound peptides upon treatment (75° C. for 5 min) with Fmoc-protected amino acid (2 mmol. 4×), 2-(1H-benzotriazol-1-yl)-1,1,3,3-tetramethyluronium hexafluorophosphate (HBTU, 2 mmol, 4×), and N,N-diisopropylethylamine (DIEA, 2 mmol, 4×) in a mixture of DMF (9 mL) and N-methyl-2-pyrrolidone (NMP, 2 mL). To remove the N-(1-(4,4-dimethyl-2,6-dioxocyclohexylidene)ethyl) (Dde) protecting group, the resin was treated with hydrazine monohydrate (2%) in DMF (3×10 min). 4-azidobutanoic acid (0.517 g, 4.0 mmol, 4×) was pre-activated upon reaction with 1-[Bis(dimethylamino)methylene]-1H-1,2,3-triazolo[4,5-b]pyridinium 3-oxid hexafluorophosphate (HATU, 1.502 g, 3.95 mmol, 3.95×) and DIEA (1.034 g, 8.0 mmol, 8×) in minimal DMF for 5 min and then reacted with the resin for 1.5 h to functionalize the ε-amino group of the C-terminal lysine with an azide. The peptide was simultaneously deprotected and cleaved from resin upon treatment with TFA/dH₂O/1,2-ethanedithiol (EDT)/triisopropylsilane (TIS) (94:2.5:2.5:1, 30 mL) for 2 h, and the crude peptide was precipitated in and washed with ice-cold diethyl ether (2×). The crude peptide was purified using RP-HPLC operating with a 55-min linear gradient (5-100%) of acetonitrile in water containing TFA (0.1%): lyophilization yielded the final product (H—SEQ ID No. 51 (N₃)—NH₂ peptide, denoted ST-N₃. SEQ ID No. 7, SEQ ID No. 8) as solid. Peptide purity was confirmed using MALDI-TOF: calculated for C₈₄H₁₃₇N₂₆O₂₂S⁺ [M+¹H−H₂O]⁺, 1894.0; observed 1893.8.

Method S20 UnaG (SEQ ID No. 41) Fluorescent Reconstitution with ST (SEQ ID No. 7, SEQ ID No. 8)-Quenched SC (SEQ ID No. 25)/pSC (SEQ ID No. 26) Fragments

pSC-cUnaG (SEQ ID No. 39) and SC-cUnaG (SEQ ID No. 37, 2 μM in Tris Buffer) were incubated with or without ST-N₃ (SEQ ID No. 7, SEQ ID No. 8, 20 μM) for 48 h at 25° C. Fluorescence reconstitution was determined by combining equimolar amounts of ST-N₃—(SEQ ID No. 7. SEQ ID No. 8) treated pSC-cUnaG (SEQ ID No. 39)/SC-cUnaG (SEQ ID No. 37) with nUnaG-ST (SEQ ID No. 31, all proteins at 1 μM) and excess bilirubin (10 μM). Reconstituted UnaG (SEQ ID No. 41) fluorescence was measured 24 h after nUnaG-ST (SEQ ID No. 31) addition using a fluorescent plate reader in 96-well, black opaque plates (λ_{excitation}=491 nm, λₑₘᵢₛₛᵢₒₙ=528 nm).

Method S21 Chemoenzymatic Synthesis of the pSC-cUnaG-N₃ (SEQ ID No. 39) Via Sortagging

pSC-cUnaG-LPETG (SEQ ID No. 40) was ordered cloned into a pET21a(+) vector at the NdeI and XhoI cloning sites (GenScript). The protein was expressed and purified as described for pSC (SEQ ID No. 26, Method S5).

SrtA_{7M} (SEQ ID No. 30) was added to pSC-cUnaG-LPETG (SEQ ID No. 40) in Tris Buffer (20 mM Tris, 50 mM NaCl, pH 7.5) at a 1:10 molar ratio and supplemented with a 100-fold molar excess of H-SEQ ID No. 50(N₃)—NH₂ peptide. After reaction (1.5 h, 37° C.). SrtA_{7M} (SEQ ID No. 30) and unreacted pSC-cUnaG-LPETG (SEQ ID No. 40) were removed through reverse IMAC purification with the addition of Ni-NTA agarose resin (Gold Biotechnology). The column flow-through containing the azide-functionalized product was buffer exchanged (MWCO˜7000 Da, Zeba Spin Desalting Column, Thermo Scientific) against PBS (pH=7.4) to remove excess peptide and generate pSC-cUnaG-N₃ (SEQ ID No. 39). Protein identity and azide-functionalization were confirmed by mass spectrometry (Table 1).

### Method S22 Photolithographically Patterned Activation of Split-UnaG (SEQ ID No. 41) Via In-Gel LASL

### Hydrogel Formation

A solution of PEG-tetraBCN (Mₙ˜20,000 Da, 4 mM) and pSC-cUnaG-N₃ (SEQ ID No. 39, 12 μM) were pre-reacted for 1 h at room temperature in PBS. PEG-diazide (Mₙ˜3,500 Da, 8 mM) crosslinker was added and the gel-precursor solution was aliquoted between Rain-X®-treated glass slides with silicone rubber spacers (0.5 mm thick, McMaster-Carr). Network formation was allowed to proceed for 1 h at room temperature before incubating gels in excess PBS overnight.

### Mask-Based Photolighography

For gels patterned via mask-based photolithography, a chrome photomask of repeating husky silhouettes (Photo Sciences) was applied between the gel and light source during exposure (λ=365 nm, 20 mW cm⁻², 15 mm). Prior to patterning, gels were incubated in a solution of nUnaG-ST (SEQ ID No. 31, 18.7 μM. PBS) overnight at room temperature. Following light exposure, excess nUnaG-ST (SEQ ID No. 31) was removed by incubating the hydrogel in PBS containing the substrate bilirubin (2 μM final concentration) before fluorescence imaging on a Stellaris 5 confocal microscope equipped with a 10× dry objective (Leica).

### Multi Photon Laser-Scanning Lithography

For gels patterned via multiphoton laser-scanning lithography, a ThorLabs Bergamo II multiphoton microscope with a 25× objective was used. Photopatterning was conducted in PBS buffer supplemented with rhodamine B (50 μM) as a two-photon sensitizer. Regions of interest were scanned through the z-dimension (30 scan repeats, pixel size=0.832 μm×0.832 μm, pixel dwell time=3200 ns, z-spacing=2 μm) with pulsed laser light (λ=740 nm, 100% laser power) to generate 3D patterns. Gels were incubated in a solution of nUnaG-ST (SEQ ID No. 31, 18.7 μM, PBS) overnight at room temperature. Excess nUnaG-ST (SEQ ID No. 31) was diffusively removed through incubation in PBS before bilirubin addition (2 μM final concentration). Fluorescence imaging was completed on a Stellaris 5 confocal microscope equipped with a 10× dry objective (Leica).

### Method S23 Molecular Cloning of Split-NanoLuc Fragments for SpyLigation and LASL

The LgBiT-ST (SEQ ID No. 43) and pSC-SmBiT (SEQ ID No. 42) fragments were ordered cloned into pET29b(+) and pET21a(+) vectors, respectively, at the NdeI and XhoI cloning sites (GenScript).

Proteins were expressed and purified as previously described (Method S5). Protein identities were confirmed by mass spectrometry (Table S1).

### Method S24 Light-Dependent Reconstitution of Split-NanoLuc Via LASL

Purified pSC-SmBiT (SEQ ID No. 42) and LgBiT-ST (SEQ ID No. 43) proteins were applied to a Zeba Spin Desalting Column (7k MWCO, 10 mL, Thermo Scientific) equilibrated with PBS Buffer (pH=7.4) to remove Tris Buffer. LASL dependent NanoLuc reconstitution of the fragments relative to concentration was determined by mixing equal molar amounts of LgBiT-ST (SEQ ID No. 43) with pSC-SmBiT (SEQ ID No. 42, 10 μM) in PBS and treating the mixture with light (λ=365 nm, 20 mW cm², 30 min) and without light. After 24 h, the NanoLuc mixture was sequentially diluted (0-1 μM) in PBS containing 0.1% BSA.

For dose-dependent studies, equal molar amounts of pSC-SmBiT (SEQ ID No. 42) and LgBiT-ST (SEQ ID No. 43, 10 μM in PBS) were combined and together treated with light (λ=365 nm, 20 mW cm⁻², 0-30 min). After 24 h. each light-exposed pSC-SmBiT (SEQ ID No. 42)/LgBiT-ST (SEQ ID No. 43) solution was diluted to 1 μM in PBS containing 0.1% BSA. NanoLuc reconstitution was determined using the Nano-Glo® Luciferase Assay System according to the manufacturer's instructions (Promega). Briefly, 50 μL of the NanoLuc fragments were added to a white-walled 96-well plate (ThermoFisher) and mixed with 24 μl of Nano-Glo® Luciferase Assay reagent. End-point luminescence readings were taken immediately in a microplate reader.

### Method S25 Design of Polycistronic Split-UnaG (SEQ ID No. 41) LASL Construct for Intracellular Activation

The SC-cUnaG-P2A-nUnaG-ST-P2A-mCh cassette was received cloned into the pTwist CMV vector with the puromycin resistance gene designed for high levels of expression in mammalian cells (Twist Bioscience). The plasmid was transformed into chemically competent Top10 E coli. Overnight cultures (10 mL Miller's LB. 100 μg mL⁻¹ carbenicillin) were pelleted at 4000×g for 10 min and plasmid DNA was collected using the QIAprep Spin Miniprep kit (Qiagen). Plasmid DNA was eluted in cell culture grade dH₂O (Corning) and used for mammalian cell transfections.

### Method S26 Mammalian Cell Culture, Transfection, and Intracellular UnaG (SEQ ID No. 41) Assembly by LASL

### General Methods

HEK-293T cells were maintained at 37° C. and 5% CO₂ in Dulbecco's minimal essential media (DMEM) supplemented with 10% FBS and 1% P/S. Cells were seeded at 50,000 cells cm⁻² on 35 mm glass bottom dishes (14 mm glass microwell size, Cellvis) coated with 0.1% gelatin 24 h prior to transfection. Upon transfection, cells were swapped to complete DMEM containing Lys(oNB)(2 mM). Equal amounts (1 μg) of the pSC-UnaG expression vector and pNeu-hMbPylRS-4×U6M15 were co-transfected using Lipofectamine 2000 following the supplied protocol.

### Light Treatment

24 h post-transfection, media was swapped with HBSS but not Lys(oNB) prior to treatment with light (λ=365 nm. 20 mW cm⁻², 0-20 min). Cells were incubated in complete DMEM without Lys(oNB) for 0-72 h before imaging. Cells were swapped to HBSS and imaged on a Stellaris 5 confocal microscope (Leica) equipped with a dry 20× objective (λ_{UnaG excitation}=489 nm, λ_{mCherry excitation}=587 nm).

### Patterned LASL UnaG (SEQ ID No. 41) Activation

To pattern intracellular UnaG (SEQ ID No. 41) assembly in a circle shape, cells were photoactivated (λ=365 nm, 20 mW cm⁻², 20 min) through an opaque rubber photomask (0.5 mm silicone rubber, McMaster-Carr) biopsy-punched to the desired hole size (7 mm). For dosage-controlled activation within a single cell population, individual culture dishes exposed regionally with varied exposures (%=365 nm, 20 mW cm⁻², 0, 3, 5, 10 min) were generated by selectively removing portions of a secondary photomask (wedge-shaped) at different time points. After light treatment, cells exposed in a circle pattern or with varied exposures were incubated in complete media without Lys(oNB) for 6 or 3 h before imaging, respectively.

### CellProfiler Analysis

Individual cell fluorescence and position was quantified using CellProfiler. Cells were first identified through mCh fluorescence via the IdentifyPrimaryObjects module. Total UnaG (SEQ ID No. 41) and mCh fluorescent signals within each cell Object was extracted for each image. UnaG (SEQ ID No. 41)/mCh and mCh ratios for each cell measurement were normalized to unexposed controls.

### Method S27 Design of Polycistronic Split-Cre LASL Construct for Intracellular Activation

The NCre-ST-NLS-P2A-NLS—SC-CCre cassette was purchased and received cloned in the pcDNA3.1 vector (GenScript). The plasmid was individually transformed into electrocompetent Top10 E. coli (ThermoFisher). Overnight cultures (10 mL Miller's LB, 100 μg mL⁻¹ carbenicillin) were pelleted at 4000×g for 10 min and plasmid DNA was collected using the QIAprep Spin Miniprep kit (Qiagen). Plasmid DNA was eluted in cell culture grade dH₂O (Corning) and used for mammalian cell transfections.

Site-directed mutagenesis was performed to generate NCre-ST-NLS-P2A-NLS-pSC-CCre (SEQ ID No. 45) from the wild-type plasmid (Method S3).

### Method S28 Primary Mammalian Cell Culture, Transfection, and Split-Cre LASL Activation

### General Methods

Transgenic mouse dermal fibroblasts bearing a Cre-dependent dual-color reporter in the “safe harbor” Rosa26 locus were maintained at 37° C. and 5% CO₂ in DMEM supplemented with 10% FBS and 1% P/S. Cells were seeded at 2500 cells cm⁻² on 35 mm glass bottom dishes (14 mm glass microwell size, Cellvis) prior to transfection. Upon transfection, cells were swapped to complete DMEM containing Lys(oNB) (2.5 mM). Equal amounts (300 ng) of the pSC-cUnaG-P2A-nUnaG-ST-P2A-mCh (SEQ ID No. 44) expression vector and pNeu-hMbPylRS-4×U6M15 were co-transfected using Lipofectamine LTX following the supplied protocol.

### Light Treatment

24 h post-transfection, media was swapped with HBSS but not Lys(oNB) prior to treatment with light (λ=365 nm, 20 mW cm⁻², 3 min). Cells were incubated in complete DMEM without Lys(oNB) for 48 h before imaging. Cells were swapped to HBSS and imaged on a Stellaris 5 confocal microscope (Leica) equipped with a dry 20× objective (λ_{EGFP excitation}=489 nm, λ_{tdTomato excitation}=554 nm).

### Patterned LASL Cre Activation

To pattern intracellular Cre assembly and targeted gene editing in a circle shape, cells were photoactivated (λ=365 nm, 20 mW cm⁻², 3 min) through an opaque rubber photomask (0.5 mm silicone rubber, McMaster-Carr) biopsy-punched to the desired hole size (2 mm). After light treatment, cells were incubated in complete media without Lys(oNB) for 48 h before imaging.

TABLE 2
Oligonucleotides used for molecular cloning

| | Primer Name | Sequence 5′ → 3′ |
|---|---|---|
| SEQ ID No. 1 | SpyCatcher-Forward | agatacgcatatggttgataccttatcaggtttatcaag |
| SEQ ID No. 2 | SpyCatcher-Reverse | cgtgcctcgagaatatgagcgtcacctttagttgc |
| SEQ ID No. 3 | SpyCatcher-SDM- | attctcatagcgtgatgaggacggcaaagagttag |
| | Forward | |
| SEQ ID No. 4 | SpyCatcher-SDM- | catcacgctatgagaatttaatatgggtagcactatcttc |
| | Reverse | |
| SEQ ID No. 5 | pSC-LPETG Forward | agatacgcatatggttgataccttatcaggtttatcaag |
| SEQ ID No. 6 | pSC-LPETG Reverse | tacgtcagctcgaggccggtttctggaagaatatgagcgtca |
| | | cctttagttgc |
| SEQ ID No. 7 | SpyTag Forward Oligo | agcttgcacacatagtaatggtagacgcctacaagccgacg |
| | | aagc |
| SEQ ID No. 8 | SpyTag Reverse Oligo | tcgagcttcgtcggcttgtaggegtctaccattactatgtgtgc |
| | | a |
| SEQ ID No. 9 | N-MBP-SpyTag Forward | gatctcgatcccgcgaaattaatac |
| SEQ ID No. 10 | N-MBP-SpyTag Reverse | gtctaccattactatgtgtgcgccaccgctcgaattagtctgc |
| | | gcgtc |
| SEQ ID No. 11 | C-SpyTag-MBP Forward | gcacgaagcttatgaaaatogaagaaggtaaactggtaatc |
| | | tgg |
| SEQ ID No. 12 | C-SpyTag-MBP Reverse | acagtctcgaggctcgaattagtctgcgcgtctttc |
| SEQ ID No. 13 | SpyTag-UnaG Forward | cagactaattcgagcggtggcgcacacatagtaatggtagac |
| SEQ ID No. 14 | SpyTag-UnaG Reverse | ccctatagtgagtcgtattaatttegcg |
| SEQ ID No. 15 | NBK-1-a Forward | tagggttttgtttaactttaagaaggagatatacttacccatat |
| | | ggataaaaagcctc |
| SEQ ID No. 16 | NBK-1-a Reverse | aaaacagccaagctggagacttacaggttagtagaaatacc |
| | | attg |
| SEQ ID No. 17 | NBK-1-b Forward | ttacgctttgaggaatcccaatggataaaaagcctctgaaca |
| | | c |
| SEQ ID No. 18 | NBK-1-b Reverse | ataagcgtagcgcatcagg |
| SEQ ID No. 19 | pEVOL Forward | gcagtttcaaacgctaaattgcctgatgc |
| SEQ ID No. 20 | pEVOL Reverse | atgggtaagtatatctccttcttaaagttaaacaaaaccctat |
| | | agtgagtc |
| SEQ ID No. 21 | ginS-Seq | actaacagttgtcagcctgtcc |
| SEQ ID No. 22 | pEVOL-REV-Seq | ataagcgtagcgcatcagg |
| SEQ ID No. 23 | hMbPylRS-NBK Forward | caccagagagaatctggaagctctgatc |
| SEQ ID No. 24 | hMbPylRS-NBK Reverse | gcctcaggcacaggtttttgtcc |

Table 3 DNA sequences for all cloned constructs

TABLE 3
DNA sequences for all cloned constructs

| Sequence ID | Primer Name | Sequence |
|---|---|---|
| SEQ ID No. 25 | SC (SC-6xHis) | atggttgataccttatcaggtttatcaagtgagcaaggt |
| | | cagtccggtgatatgacaattgaagaagatagtgctac |
| | | ccatattaaattctcaaaacgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattctcga |
| | | gcaccaccaccaccaccactga |
| SEQ ID No. 26 | pSC (pSC-6xHis) | atggttgataccttatcaggtttatcaagtgagcaaggt |
| | | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattctcga |
| | | gcaccaccaccaccaccactga |
| SEQ ID No. 27 | pSC-LPETG (pSC-LPETG- | atggttgataccttatcaggtttatcaagtgagcaaggt |
| | 6xHis) | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattcttcca |
| | | gaaaccggcctcgagcaccaccaccaccaccactga |
| SEQ ID No. 28 | GST-ST [Glutathione-S- | atgtcccctatactaggttattggaaaattaagggccttg |
| | transferase-(GGS)x5-ST] | tgcaacccactcgacttcttttggaatatcttgaagaaa |
| | | aatatgaagagcatttgtatgagcgcgatgaaggtgat |
| | | aaatggcgaaacaaaaagtttgaattgggtttggagttt |
| | | cccaatcttccttattatattgatggtgatgttaaattaac |
| | | acagtctatggccatcatacgttatatagctgacaagca |
| | | caacatgttgggtggttgtccaaaagagcgtgcagaga |
| | | tttcaatgcttgaaggagcggttttggatattagatacgg |
| | | tgtttcgagaattgcatatagtaaagactttgaaactctc |
| | | aaagttgattttcttagcaagctacctgaaatgctgaaa |
| | | atgttcgaagatcgtttatgtcataaaacatatttaaatg |
| | | gtgatcatgtaacccatcctgacttcatgttgtatgacgc |
| | | tcttgatgttgttttatacatggacccaatgtgcctggat |
| | | gcgttcccaaaattagtttgttttaaaaaacgtattgaag |
| | | ctatcccacaaattgataagtacttgaaatccagcaagt |
| | | atatagcatggcctttgcagggctggcaagccacgtttg |
| | | gtggtggcgaccatcctccaaaatcggatctggttccgc |
| | | gtggatccggtggttctggtggttctggtggttctggtgg |
| | | ttctggtggttctgctcacatcgttatggttgacgcttaca |
| | | aaccgaccaaatga |
| SEQ ID No. 29 | mRuby-ST (mRuby-ST-6xHis) | atggtttctaaaggtgaagaactgatcaaagaaaacat |
| | | gcgtatgaaagttgttatggaaggttctgttaacggtca |
| | | ccagttcaaatgcaccggtgaaggtgaaggtcgtccgt |
| | | acgaaggtgttcagaccatgcgtatcaaagttatcgaa |
| | | ggtggtccgctgccgttcgctttcgacatcctggctacct |
| | | ctttcatgtacggttctcgtaccttcatcaaatacccggc |
| | | tgacatcccggacttcttcaaacagtctttcccggaaggt |
| | | ttcacctgggaacgtgttacccgttacgaagacggtggt |
| | | gttgttaccgttacccaggacacctctctggaagacggt |
| | | gaactggtttacaacgttaaagttcgtggtgttaacttcc |
| | | cgtctaacggtccggttatgcagaaaaaaaccaaaggt |
| | | tgggaaccgaacaccgaaatgatgtacccggctgacgg |
| | | tggtctgcgtggttacaccgacatcgctctgaaagttga |
| | | cggtggtggtcacctgcactgcaacttcgttaccaccta |
| | | ccgttctaaaaaaaccgttggtaacatcaaaatgccgg |
| | | gtgttcacgctgttgaccaccgtctggaacgtatcgaag |
| | | aatctgacaacgaaacctacgttgttcagcgtgaagttg |
| | | ctgttgctaaatactctaacctgggtggtggtatggacg |
| | | aactgtacaaaaagcttgcacacatagtaatggtagac |
| | | gcctacaagccgacgaagctcgagcaccaccaccacc |
| | | accactga |
| SEQ ID No. 30 | SrtA_{7M} (Sortase 7M-6xHis) | atgcaagctaaacctcaaattccgaaagataaatcaaa |
| | | agtggcaggctatattgaaattccagatgctgatattaa |
| | | agaaccagtatatccaggaccagcaacacgcgaacaa |
| | | ttaaatagaggtgtaagctttgcaaaagaaaatcaatc |
| | | actagatgatcaaaatatttcaattgcaggacacacttt |
| | | cattgaccgtccgaactatcaatttacaaatcttaaagc |
| | | agccaaaaaaggtagtatggtgtactttaaagttggta |
| | | atgaaacacgtaagtataaaatgacaagtataagaaa |
| | | cgttaagccaacagctgtagaagttctagatgaacaaa |
| | | aaggtaaagataaacaattaacattaattacttgtgatg |
| | | attacaatgaagagacaggcgtttgggaaacacgtaaa |
| | | atctttgtagctacagaagtcaaactcgagcaccaccac |
| | | caccaccactga |
| SEQ ID No. 31 | nUnaG-ST (nUnaG-SEQ ID. | atggttgaaaaatttgtgggtacgtggaaaattgcggat |
| | No. 52-ST-MBP-6xHis) | tcgcataattttggcgaatacctgaaagctatcggggca |
| | | cctaaagaactgagtgacggaggcgatgccacgaccc |
| | | caactctttatatttcccagaaggacggtgataaaatga |
| | | ccgtaaagatcgagaacggcccgcccaccttcttagat |
| | | acacaagtcaaatttaaattgggtgaagaattcgatga |
| | | gttcccgagcgaccgtcgcaaaggtggctctggaggtg |
| | | gatctggcggttctgcacacatagtaatggtagacgcct |
| | | acaagccgacgaagaagcttatgaaaatcgaagaagg |
| | | taaactggtaatctggattaacggcgataaaggctata |
| | | acggtctcgctgaagtcggtaagaaattcgagaaagat |
| | | accggaattaaagtcaccgttgagcatccggataaact |
| | | ggaagagaaattcccacaggttgcggcaactggcgatg |
| | | gccctgacattatcttctgggcacacgaccgctttggtgg |
| | | ctacgctcaatctggcctgttggctgaaatcaccccgga |
| | | caaagcgttccaggacaagctgtatccgtttacctggga |
| | | tgccgtacgttacaacggcaagctgattgcttacccgat |
| | | cgctgttgaagcgttatcgctgatttataacaaagatctg |
| | | ctgccgaacccgccaaaaacctgggaagagatcccgg |
| | | cgctggataaagaactgaaagcgaaaggtaagagcgc |
| | | gctgatgttcaacctgcaagaaccgtacttcacctggcc |
| | | gctgattgctgctgacgggggttatgcgttcaagtatga |
| | | aaacggcaagtacgacattaaagacgtgggcgtggat |
| | | aacgctggcgcgaaagcgggtctgaccttcctggttgac |
| | | ctgattaaaaacaaacacatgaatgcagacaccgatta |
| | | ctccatcgcagaagctgcctttaataaaggcgaaacag |
| | | cgatgaccatcaacggcccgtgggcatggtccaacatc |
| | | gacaccagcaaagtgaattatggtgtaacggtactgcc |
| | | gaccttcaagggtcaaccatccaaaccgttcgttggcgt |
| | | gctgagcgcaggtattaacgccgccagtccgaacaaag |
| | | agctggcaaaagagttcctcgaaaactatctgctgactg |
| | | atgaaggtctggaagcggttaataaagacaaaccgctg |
| | | ggtgccgtagcgctgaagtcttacgaggaagagttggc |
| | | gaaagatccacgtattgccgccaccatggaaaacgccc |
| | | agaaaggtgaaatcatgccgaacatcccgcagatgtcc |
| | | gctttctggtatgccgtgcgtactgcggtgatcaacgcc |
| | | gccagcggtcgtcagactgtcgatgaagccctgaaaga |
| | | cgcgcagactaattcgagcctcgagcaccaccaccacc |
| | | accactga |
| SEQ ID No. 32 | ST-nUnaG (MBP-ST-SEQ ID | atgaaaatcgaagaaggtaaactggtaatctggattaa |
| | No. 53-nUnaG-6xHis) | cggcgataaaggctataacggtctcgctgaagtcggta |
| | | agaaattcgagaaagataccggaattaaagtcaccgtt |
| | | gagcatccggataaactggaagagaaattcccacaggt |
| | | tgcggcaactggcgatggccctgacattatcttctgggc |
| | | acacgaccgctttggtggctacgctcaatctggcctgttg |
| | | gctgaaatcaccccggacaaagcgttccaggacaagct |
| | | gtatccgtttacctgggatgccgtacgttacaacggcaa |
| | | gctgattgcttacccgatcgctgttgaagcgttatcgctg |
| | | atttataacaaagatctgctgccgaacccgccaaaaac |
| | | ctgggaagagatcccggcgctggataaagaactgaaa |
| | | gcgaaaggtaagagcgcgctgatgttcaacctgcaaga |
| | | accgtacttcacctggccgctgattgctgctgacggggg |
| | | ttatgcgttcaagtatgaaaacggcaagtacgacattaa |
| | | agacgtgggcgtggataacgctggcgcgaaagcgggt |
| | | ctgaccttcctggttgacctgattaaaaacaaacacatg |
| | | aatgcagacaccgattactccatcgcagaagctgccttt |
| | | aataaaggcgaaacagcgatgaccatcaacggcccgt |
| | | gggcatggtccaacatcgacaccagcaaagtgaattat |
| | | ggtgtaacggtactgccgaccttcaagggtcaaccatcc |
| | | aaaccgttcgttggcgtgctgagcgcaggtattaacgcc |
| | | gccagtccgaacaaagagctggcaaaagagttcctcga |
| | | aaactatctgctgactgatgaaggtctggaagcggttaa |
| | | taaagacaaaccgctgggtgccgtagcgctgaagtctt |
| | | acgaggaagagttggcgaaagatccacgtattgccgcc |
| | | accatggaaaacgcccagaaaggtgaaatcatgccga |
| | | acatcccgcagatgtccgctttctggtatgccgtgcgtac |
| | | tgcggtgatcaacgccgccagcggtcgtcagactgtcg |
| | | atgaagccctgaaagacgcgcagactaattcgagcggt |
| | | ggcgcacacatagtaatggtagacgcctacaagccgac |
| | | gaagggtggctctggaggtggatctggcatggttgaaa |
| | | aatttgtgggtacgtggaaaattgcggattcgcataattt |
| | | tggcgaatacctgaaagctatcggggcacctaaagaac |
| | | tgagtgacggaggcgatgccacgaccccaactctttata |
| | | tttcccagaaggacggtgataaaatgaccgtaaagatc |
| | | gagaacggcccgcccaccttcttagatacacaagtcaa |
| | | atttaaattgggtgaagaattcgatgagttcccgagcga |
| | | ccgtcgcaaaaagcttggtaccctcgagcaccaccacc |
| | | accaccactga |
| SEQ ID No. 33 | cUnaG-ST (cUnaG-SEQ ID No. | atgtccggtgttaaatccgtagtcaacttggtcggagaa |
| | 54-ST-MBP-6xHis) | aaactcgtatatgtccaaaaatgggatggcaaagagac |
| | | aacctacgtgcgtgaaattaaagacgggaaactggttg |
| | | tgacgctgacaatgggcgacgtggtggcagtgcgctcg |
| | | taccggcgtgcaaccgaaggtggctctggaggtggatc |
| | | tggcggttctgcacacatagtaatggtagacgcctacaa |
| | | gccgacgaagaagcttatgaaaatcgaagaaggtaaa |
| | | ctggtaatctggattaacggcgataaaggctataacggt |
| | | ctcgctgaagtcggtaagaaattcgagaaagataccgg |
| | | aattaaagtcaccgttgagcatccggataaactggaag |
| | | agaaattcccacaggttgcggcaactggcgatggccct |
| | | gacattatcttctgggcacacgaccgctttggtggctac |
| | | gctcaatctggcctgttggctgaaatcaccccggacaaa |
| | | gcgttccaggacaagctgtatccgtttacctgggatgcc |
| | | gtacgttacaacggcaagctgattgcttacccgatcgct |
| | | gttgaagcgttatcgctgatttataacaaagatctgctgc |
| | | cgaacccgccaaaaacctgggaagagatcccggcgct |
| | | ggataaagaactgaaagcgaaaggtaagagcgcgctg |
| | | atgttcaacctgcaagaaccgtacttcacctggccgctg |
| | | attgctgctgacgggggttatgcgttcaagtatgaaaac |
| | | ggcaagtacgacattaaagacgtgggcgtggataacgc |
| | | tggcgcgaaagcgggtctgaccttcctggttgacctgat |
| | | taaaaacaaacacatgaatgcagacaccgattactcca |
| | | tcgcagaagctgcctttaataaaggcgaaacagcgatg |
| | | accatcaacggcccgtgggcatggtccaacatcgacac |
| | | cagcaaagtgaattatggtgtaacggtactgccgacctt |
| | | caagggtcaaccatccaaaccgttcgttggcgtgctgag |
| | | cgcaggtattaacgccgccagtccgaacaaagagctgg |
| | | caaaagagttcctcgaaaactatctgctgactgatgaa |
| | | ggtctggaagcggttaataaagacaaaccgctgggtgc |
| | | cgtagcgctgaagtcttacgaggaagagttggcgaaag |
| | | atccacgtattgccgccaccatggaaaacgcccagaaa |
| | | ggtgaaatcatgccgaacatcccgcagatgtccgctttc |
| | | tggtatgccgtgcgtactgcggtgatcaacgccgccagc |
| | | ggtcgtcagactgtcgatgaagccctgaaagacgcgca |
| | | gactaattcgagcctcgagcaccaccaccaccaccact |
| | | ga |
| SEQ ID No. 34 | ST-cUnaG (MBP-ST-SEQ ID | atgaaaatcgaagaaggtaaactggtaatctggattaa |
| | No. 53-cUnaG-6xHis) | cggcgataaaggctataacggtctcgctgaagtcggta |
| | | agaaattcgagaaagataccggaattaaagtcaccgtt |
| | | gagcatccggataaactggaagagaaattcccacaggt |
| | | tgcggcaactggcgatggccctgacattatcttctgggc |
| | | acacgaccgctttggtggctacgctcaatctggcctgttg |
| | | gctgaaatcaccccggacaaagcgttccaggacaagct |
| | | gtatccgtttacctgggatgccgtacgttacaacggcaa |
| | | gctgattgcttacccgatcgctgttgaagcgttatcgctg |
| | | atttataacaaagatctgctgccgaacccgccaaaaac |
| | | ctgggaagagatcccggcgctggataaagaactgaaa |
| | | gcgaaaggtaagagcgcgctgatgttcaacctgcaaga |
| | | accgtacttcacctggccgctgattgctgctgacggggg |
| | | ttatgcgttcaagtatgaaaacggcaagtacgacattaa |
| | | agacgtgggcgtggataacgctggcgcgaaagcgggt |
| | | ctgaccttcctggttgacctgattaaaaacaaacacatg |
| | | aatgcagacaccgattactccatcgcagaagctgccttt |
| | | aataaaggcgaaacagcgatgaccatcaacggcccgt |
| | | gggcatggtccaacatcgacaccagcaaagtgaattat |
| | | ggtgtaacggtactgccgaccttcaagggtcaaccatcc |
| | | aaaccgttcgttggcgtgctgagcgcaggtattaacgcc |
| | | gccagtccgaacaaagagctggcaaaagagttcctcga |
| | | aaactatctgctgactgatgaaggtctggaagcggttaa |
| | | taaagacaaaccgctgggtgccgtagcgctgaagtctt |
| | | acgaggaagagttggcgaaagatccacgtattgccgcc |
| | | accatggaaaacgcccagaaaggtgaaatcatgccga |
| | | acatcccgcagatgtccgctttctggtatgccgtgcgtac |
| | | tgcggtgatcaacgccgccagcggtcgtcagactgtcg |
| | | atgaagccctgaaagacgcgcagactaattcgagcggt |
| | | ggcgcacacatagtaatggtagacgcctacaagccgac |
| | | gaagggtggcagtggtggtgggagcggtcggtgttaaa |
| | | tccgtagtcaacttggtcggagaaaaactcgtatatgtc |
| | | caaaaatgggatggcaaagagacaacctacgtgcgtg |
| | | aaattaaagacgggaaactggttgtgacgctgacaatg |
| | | ggcgacgtggtggcagtgcgctcgtaccggcgtgcaac |
| | | cgaaaagcttggtaccctcgagcaccaccaccaccacc |
| | | actga |
| SEQ ID No. 35 | SC-nUnaG (SC-SEQ ID No. 53- | atggttgataccttatcaggtttatcaagtgagcaaggt |
| | nUnaG-6xHis) | cagtccggtgatatgacaattgaagaagatagtgctac |
| | | ccatattaaattctcaaaacgtgatgaggacggcaaag |
| | | agttagctggtgcaactatggagttgcgtgattcatctg |
| | | gtaaaactattagtacatggatttcagatggacaagtga |
| | | aagatttctacctgtatccaggasaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | ctctggaggtggatctggcatggttgaaaaatttgtggg |
| | | tacgtggaaaattgcggattcgcataattttggcgaata |
| | | cctgaaagctatcggggcacctaaagaactgagtgacg |
| | | gaggcgatgccacgaccccaactctttatatttcccaga |
| | | aggacggtgataaaatgaccgtaaagatcgagaacgg |
| | | cccgcccaccttcttagatacacaagtcaaatttaaatt |
| | | gggtgaagaattcgatgagttcccgagcgaccgtcgca |
| | | aaggtggcagtctcgagcaccaccaccaccaccactga |
| SEQ ID No. 36 | nUnaG-SC (nUnaG-SEQ ID | atggttgaaaaatttgtgggtacgtggaaaattgcggat |
| | No. 54-SC-6xHis) | tcgcataattttggcgaatacctgaaagctatcggggca |
| | | cctaaagaactgagtgacggaggcgatgccacgaccc |
| | | caactctttatatttcccagaaggacggtgataaaatga |
| | | ccgtaaagatcgagaacggcccgcccaccttcttagat |
| | | acacaagtcaaatttaaattgggtgaagaattcgatga |
| | | gttcccgagcgaccgtcgcaaaggtggctctggaggtg |
| | | gatctggcggttctatggttgataccttatcaggtttatc |
| | | aagtgagcaaggtcagtccggtgatatgacaattgaag |
| | | aagatagtgctacccatattaaattctcaaaacgtgatg |
| | | aggacggcaaagagttagctggtgcaactatggagttg |
| | | cgtgattcatctggtaaaactattagtacatggatttcag |
| | | atggacaagtgaaagatttctacctgtatccaggaaaat |
| | | atacatttgtcgaaaccgcagcaccagacggttatgag |
| | | gtagcaactgctattacctttacagttaatgagcaaggt |
| | | caggttactgtaaatggcaaagcaactaaaggtgacgc |
| | | tcatattaagcttggtaccctcgagcaccaccaccacca |
| | | ccactga |
| SEQ ID No. 37 | SC-cUnaG (SC-SEQ ID No. 55- | atggttgataccttatcaggtttatcaagtgagcaaggt |
| | cUnaG-6xHis) | cagtccggtgatatgacaattgaagaagatagtgctac |
| | | ccatattaaattctcaaaacgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | cagtggtggtgggagcggcggtgttaaatccgtagtca |
| | | acttggtcggagaaaaactcgtatatgtccaaaaatgg |
| | | gatggcaaagagacaacctacgtgcgtgaaattaaag |
| | | acgggaaactggttgtgacgctgacaatgggcgacgtg |
| | | gtggcagtgcgctcgtaccggcgtgcaaccgaaaagct |
| | | tggtaccctcgagcaccaccaccaccaccactga |
| SEQ ID No. 38 | cUnaG-SC (cUnaG-SEQ ID No. | atgtccggtgttaaatccgtagtcaacttggtcggagaa |
| | 52-SC-6xHis) | aaactcgtatatgtccaaaaatgggatggcaaagagac |
| | | aacctacgtgcgtgaaattaaagacgggaaactggttg |
| | | tgacgctgacaatgggcgacgtggtggcagtgcgctcg |
| | | taccggcgtgcaaccgaaggtggctctggaggtggatc |
| | | tggcggttctatggttgataccttatcaggtttatcaagt |
| | | gagcaaggtcagtccggtgatatgacaattgaagaaga |
| | | tagtgctacccatattaaattctcaaaacgtgatgagga |
| | | cggcaaagagttagctggtgcaactatggagttgcgtg |
| | | attcatctggtaaaactattagtacatggatttcagatgg |
| | | acaagtgaaagatttctacctgtatccaggaaaatatac |
| | | atttgtcgaaaccgcagcaccagacggttatgaggtag |
| | | caactgctattacctttacagttaatgagcaaggtcagg |
| | | ttactgtaaatggcaaagcaactaaaggtgacgctcat |
| | | attaagcttggtaccctcgagcaccaccaccaccaccac |
| | | tga |
| SEQ ID No. 39 | pSC-cUnaG (pSC-SEQ ID No. | atggttgataccttatcaggtttatcaagtgagcaaggt |
| | 55-cUnaG-6xHis) | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | cagtggtgggggagcggcggtgttaaatccgtagtca |
| | | acttggtcggagaaaaactcgtatatgtccaaaaatgg |
| | | gatggcaaagagacaacctacgtgcgtgaaattaaag |
| | | acgggaaactggttgtgacgctgacaatgggcgacgtg |
| | | gtggcagtgcgctcgtaccggcgtgcaaccgaaaagct |
| | | tggtaccctcgagcaccaccaccaccaccactga |
| SEQ ID No. 40 | 0 pSC-cUnaG-LPETG (pSC-SEQ | atggttgataccttatcaggtttatcaagtgagcaaggt |
| | ID No. 53-cUnaG-LPETG- | cagtccggtgatatgacaattgaagaagatagtgctac |
| | 6xHis) | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | cagtggtggtgggagcggcggtgttaaatccgtagtca |
| | | acttggtcggagaaaaactcgtatatgtccaaaaatgg |
| | | gatggcaaagagacaacctacgtgcgtgaaattaaag |
| | | acgggaaactggttgtgacgctgacaatgggcgacgtg |
| | | gtggcagtgcgctcgtaccggcgtgcaaccgaacttcc |
| | | agaaaccggcctcgagcaccaccaccaccaccactga |
| SEQ ID No. 41 | UnaG (UnaG-6xHis) | atggtggaaaagttcgttggtacctggaaaatogctgat |
| | | agccataattttggtgaatatctgaaggcgattggcgct |
| | | ccgaaagaactgagcgatggtggcgacgctaccacccc |
| | | gaccctgtacatcagccagaaagatggtgacaagatga |
| | | ccgtgaaaattgaaaatggtccgccgaccttcctggata |
| | | cccaagttaaatttaagctgggtgaagaattcgatgaat |
| | | ttccgagcgaccgtcgtaaaggtgtgaagagcgtggtt |
| | | aacctggttggcgaaaagctggtgtatgttcagaagtg |
| | | ggacggcaaagaaaccacctacgtgcgtgaaatcaaa |
| | | gatggtaaactggtggtgaccctgaccatgggcgacgt |
| | | ggttgcagttcgtagctatcgtcgtgcgaccgaacatca |
| | | ccatcaccatcac |
| SEQ ID No. 42 | pSC-SmBiT (pSC-SEQ ID No. | atggttgataccttatcaggtttatcaagtgagcaaggt |
| | 53-SmBiT-6xHis) | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | cagtggtggtgggagcggcgtgactggatacagactttt |
| | | tgaagagatcctggaattcgggggtggaggttcactcg |
| | | agcaccaccaccaccaccactga |
| SEQ ID No. 43 | LgBiT-ST (LgBiT-SEQ ID No. | atggtcttcacactcgaagatttcgttggggactgggaa |
| | 54-ST-6xHis) | cagacagccgcgtacaacctggaccaagtccttgaaca |
| | | gggaggtgtgtccagtttgctgcagaatctcgcggtgtc |
| | | cgtaactccgatccaaaggattgtccgcagcggtgaaa |
| | | atgcgctgaagatcgacatccatgtcatcatcccgtatg |
| | | aaggtctgagcgcggaccaaatggcacagatcgaaga |
| | | agtgtttaaggtggtgtaccctgtggatgatcatcacttt |
| | | aaggtgatcctgccgtatggcacactggtaatcgacgg |
| | | ggttacgccgaacatgctgaactatttcggacggccgta |
| | | tgaaggcatcgccgtgttcgacggcaaaaagatcactg |
| | | taacagggaccctgtggaacggcaacaaaattatcgac |
| | | gagcgcctgatcacccccgacggctccatgctgttccga |
| | | gtaaccatcaacagcggtggctctggaggtggatctgg |
| | | cggttctgcacacatagtaatggtagacgcctacaagcc |
| | | gacgaagctcgagcaccaccaccaccaccactga |
| SEQ ID No. 44 | pSC-cUnaG-P2A-nUnaG-ST- | atggttgataccttatcaggtttatcaagtgagcaaggt |
| | P2A-mCh | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggtgg |
| | | ctctggaggtggatctggcggtgttaaatccgtagtcaa |
| | | cttggtcggagaaaaactcgtatatgtccaaaaatggg |
| | | atggcaaagagacaacctacgtgcgtgaaattaaaga |
| | | cgggaaactggttgtgacgctgacaatgggcgacgtgg |
| | | tggcagtgcgctcgtaccggcgtgcaaccgaaggaagc |
| | | ggtgccaccaatttctccctcctgaagcaggctggggac |
| | | gtggaggaaaaccctggccccatggttgaaaaatttgtt |
| | | ggtacgtggaaaatagcggattctcataattttggcgaa |
| | | tatctgaaggctatcggggcgcctaaagagctgagtga |
| | | tggaggcgatgccacgactccaactctgtatatttcaca |
| | | gaaggacggtgataaaatgaccgttaagatcgagaat |
| | | ggcccgcccaccttcctggatacacaggtaaagtttaaa |
| | | cttggtgaagaatttgatgagttcccgagcgacagacgc |
| | | aaaggaggctctggtggaggctctggaggtggctctgg |
| | | agcacacatagtaatggtagacgcctacaagccgacg |
| | | aagggaagcggagctactaacttcagcctgctgaagca |
| | | ggctggagacgtggaggagaaccctggacctgtgagc |
| | | aagggcgaggaggataacatggccatcatcaaggagt |
| | | tcatgcgcttcaaggtgcacatggagggctccgtgaac |
| | | ggccacgagttcgagatcgagggcgagggcgagggcc |
| | | gcccctacgagggcacccagaccgccaagctgaaggt |
| | | gaccaagggggccccctgcccttcgcctgggacatcct |
| | | gtcccctcagttcatgtacggctccaaggcctacgtgaa |
| | | gcaccccgccgacatccccgactacttgaagctgtcctt |
| | | ccccgagggcttcaagtgggagcgcgtgatgaacttcg |
| | | aggacggcggcgtggtgaccgtgacccaggactcctcc |
| | | ctccaggacggcgagttcatctacaaggtgaagctgcg |
| | | cggcaccaacttcccctccgacggccccgtaatgcaga |
| | | agaagaccatgggctgggaggcctcctccgagcggatg |
| | | taccccgaggacggcgccctgaagggcgagatcaagc |
| | | agaggctgaagctgaaggacggcggccactacgacgc |
| | | tgaggtcaagaccacctacaaggccaagaagcccgtg |
| | | cagctgcccggcgcctacaacgtcaacatcaagttgga |
| | | catcacctcccacaacgaggactacaccatcgtggaac |
| | | agtacgaacgcgccgagggccgccactccaccggcgg |
| | | catggacgagctgtacaagtaa |
| SEQ ID No. 45 | nCre-ST-NLS-P2A-NLS-pSC- | atggccacctctgatgaagtcaggaagaacctgatgga |
| | cCre | catgttcagggacaggcaggccttctctgaacacacctg |
| | | gaagatgctcctgtctgtgtgcagatcctgggctgcctg |
| | | gtgcaagctgaacagcggcggcagcgggagtacagca |
| | | cacatagtaatggtagacgcctacaagccgacgaaggt |
| | | gcccaagaagaagaggaaagtcggaagcggtgccacc |
| | | aatttctccctcctgaagcaggctggggacgtggagga |
| | | aaaccctggccccgtgcccaagaagaagaggaaagtc |
| | | ggttccatggttgataccttatcaggtttatcaagtgagc |
| | | aaggtcagtccggtgatatgacaattgaagaagatagt |
| | | gctacccatattaaattctcatagcgtgatgaggacggc |
| | | aaagagttagctggtgcaactatggagttgcgtgattca |
| | | tctggtaaaactattagtacatggatttcagatggacaa |
| | | gtgaaagatttctacctgtatccaggaaaatatacattt |
| | | gtcgaaaccgcagcaccagacggttatgaggtagcaac |
| | | tgctattacctttacagttaatgagcaaggtcaggttact |
| | | gtaaatggcaaagcaactaaaggtgacgctcatatttct |
| | | ggtggcagtggaagcactaacaggaaatggttccctgc |
| | | tgaacctgaggatgtgagggactacctcctgtacctgca |
| | | agccagaggcctggctgtgaagaccatccaacagcacc |
| | | tgggccagctcaacatgctgcacaggagatctggcctg |
| | | cctcgcccttctgactccaatgctgtgtccctggtgatga |
| | | ggagaatcagaaaggagaatgtggatgctggggagag |
| | | agccaagcaggccctggcctttgaacgcactgactttga |
| | | ccaagtcagatccctgatggagaactctgacagatgcc |
| | | aggacatcaggaacctggccttcctgggcattgcctaca |
| | | acaccctgctgcgcattgccgaaattgccagaatcaga |
| | | gtgaaggacatctcccgcaccgatggtgggagaatgct |
| | | gatccacattggcaggaccaagaccctggtgtccacag |
| | | ctggtgtggagaaggccctgtccctgggggttaccaag |
| | | ctggtggagagatggatctctgtgtctggtgtggctgat |
| | | gaccccaacaactacctgttctgccgggtcagaaagaa |
| | | tggtgtggctgccccttctgccacctcccaactgtccacc |
| | | cgggccctggaagggatctttgaggccacccaccgcct |
| | | gatctatggtgccaaggatgactctgggcagagatacct |
| | | ggcctggtctggccactctgccagagtgggtgctgccag |
| | | ggacatggccagggctggtgtgtccatccctgaaatcat |
| | | gcaggctggtggctggaccaatgtgaacattgtgatga |
| | | actacatcagaaacctggactctgagactggggccatg |
| | | gtgaggctgctcgaggat |
| SEQ ID No. 46 | EGFP-ST-P2A-pSC-CAAX | gtgagcaagggcgaggagctgttcaccggggtggtgcc |
| | | catcctggtcgagctggacggcgacgtaaacggccaca |
| | | agttcagcgtgtccggcgagggcgagggcgatgccacc |
| | | tacggcaagctgaccctgaagttcatctgcaccaccggc |
| | | aagctgcccgtgccctggcccaccctcgtgaccaccctg |
| | | acctacggcgtgcagtgcttcagccgctaccccgaccac |
| | | atgaagcagcacgacttcttcaagtccgccatgcccga |
| | | aggctacgtccaggagcgcaccatcttcttcaaggacg |
| | | acggcaactacaagacccgcgccgaggtgaagttcga |
| | | gggcgacaccctggtgaaccgcatcgagctgaagggc |
| | | atcgacttcaaggaggacggcaacatcctggggcacaa |
| | | gctggagtacaactacaacagccacaacgtctatatca |
| | | tggccgacaagcagaagaacggcatcaaggtgaactt |
| | | caagatccgccacaacatcgaggacggcagcgtgcag |
| | | ctcgccgaccactaccagcagaacacccccatcggcga |
| | | cggccccgtgctgctgcccgacaaccactacctgagca |
| | | cccagtccgccctgagcaaagaccccaacgagaagcg |
| | | cgatcacatggtcctgctggagttcgtgaccgccgccgg |
| | | gatcactctcggcatggacgagctgtacaaggggggaa |
| | | gtggagggagtggggggagcggggcacacatagtaat |
| | | ggtagacgcctacaagccgacgaagggaagcggagct |
| | | actaacttcagcctgctgaagcaggctggagacgtgga |
| | | ggagaaccctggacctatggttgataccttatcaggttt |
| | | atcaagtgagcaaggtcagtccggtgatatgacaattg |
| | | aagaagatagtgctacccatattaaattctcatagcgtg |
| | | atgaggacggcaaagagttagctggtgcaactatggag |
| | | ttgcgtgattcatctggtaaaactattagtacatggattt |
| | | cagatggacaagtgaaagatttctacctgtatccagga |
| | | aaatatacatttgtcgaaaccgcagcaccagacggttat |
| | | gaggtagcaactgctattacctttacagttaatgagcaa |
| | | ggtcaggttactgtaaatggcaaagcaactaaaggtga |
| | | cgctcatattggtggatctggaggtggatctggaggtat |
| | | gagcaaagatggcaaaaagaagaaaaagaagtcaaa |
| | | gacaaagtgtgtaattatg |
| SEQ ID No. 47 | pSC-mCh-P2A-ST-EGFP-CAAX | atggttgataccttatcaggtttatcaagtgagcaaggt |
| | | cagtccggtgatatgacaattgaagaagatagtgctac |
| | | ccatattaaattctcatagcgtgatgaggacggcaaag |
| | | agttagctggtgcaactatggagttgcgtgattcatctg |
| | | gtaaaactattagtacatggatttcagatggacaagtga |
| | | aagatttctacctgtatccaggaaaatatacatttgtcg |
| | | aaaccgcagcaccagacggttatgaggtagcaactgct |
| | | attacctttacagttaatgagcaaggtcaggttactgta |
| | | aatggcaaagcaactaaaggtgacgctcatattggggg |
| | | aagtggagggagtggggggagcggggtgagcaaggg |
| | | cgaggaggataacatggccatcatcaaggagttcatgc |
| | | gcttcaaggtgcacatggagggctccgtgaacggccac |
| | | gagttcgagatcgagggcgagggcgagggccgcccct |
| | | acgagggcacccagaccgccaagctgaaggtgaccaa |
| | | gggtggccccctgcccttcgcctgggacatcctgtcccct |
| | | cagttcatgtacggctccaaggcctacgtgaagcacccc |
| | | gccgacatccccgactacttgaagctgtccttccccgag |
| | | ggcttcaagtgggagcgcgtgatgaacttcgaggacgg |
| | | cggcgtggtgaccgtgacccaggactcctccctccagg |
| | | acggcgagttcatctacaaggtgaagctgcgcggcacc |
| | | aacttcccctccgacggccccgtaatgcagaagaagac |
| | | catgggctgggaggcctcctccgagcggatgtaccccg |
| | | aggacggcgccctgaagggcgagatcaagcagaggct |
| | | gaagctgaaggacggcggccactacgacgctgaggtc |
| | | aagaccacctacaaggccaagaagcccgtgcagctgc |
| | | ccggcgcctacaacgtcaacatcaagttggacatcacct |
| | | cccacaacgaggactacaccatcgtggaacagtacga |
| | | acgcgccgagggccgccactccaccggcggcatggac |
| | | gagctgtacaagggaagcggagctactaacttcagcct |
| | | gctgaagcaggctggagacgtggaggagaaccctgga |
| | | cctgcacacatagtaatggtagacgcctacaagccgac |
| | | gaaggggggaagtggagggagtggggggagcggggt |
| | | gagcaagggcgaggagctgttcaccggggtggtgccca |
| | | tcctggtcgagctggacggcgacgtaaacggccacaag |
| | | ttcagcgtgtccggcgagggcgagggcgatgccaccta |
| | | cggcaagctgaccctgaagttcatctgcaccaccggca |
| | | agctgcccgtgccctggcccaccctcgtgaccaccctga |
| | | cctacggcgtgcagtgcttcagccgctaccccgaccaca |
| | | tgaagcagcacgacttcttcaagtccgccatgcccgaa |
| | | ggctacgtccaggagcgcaccatcttcttcaaggacga |
| | | cggcaactacaagacccgcgccgaggtgaagttcgag |
| | | ggcgacaccctggtgaaccgcatcgagctgaagggcat |
| | | cgacttcaaggaggacggcaacatcctggggcacaag |
| | | ctggagtacaactacaacagccacaacgtctatatcat |
| | | ggccgacaagcagaagaacggcatcaaggtgaacttc |
| | | aagatccgccacaacatcgaggacggcagcgtgcagc |
| | | tcgccgaccactaccagcagaacacccccatcggcgac |
| | | ggccccgtgctgctgcccgacaaccactacctgagcac |
| | | ccagtccgccctgagcaaagaccccaacgagaagcgc |
| | | gatcacatggtcctgctggagttcgtgaccgccgccggg |
| | | atcactctcggcatggacgagctgtacaagggtggatct |
| | | ggaggtggatctggaggtatgagcaaagatggcaaaa |
| | | agaagaaaaagaagtcaaagacaaagtgtgtaattat |
| | | g |
| SEQ ID No. 48 | rrnB-glnS | gtatttctactaacctgtaagtctccagcttggctgttttg |
| | | gcggatgagagaagattttcagcctgatacagattaaa |
| | | tcagaacgcagaagcggtctgataaaacagaatttgcc |
| | | tggcggcagtagcgcggtggtcccacctgaccccatgc |
| | | cgaactcagaagtgaaacgccgtagcgccgatggtagt |
| | | gtggggtctccccatgcgagagtagggaactgccaggc |
| | | atcaaataaaacgaaaggctcagtcgaaagactgggc |
| | | ctttcgttttatctgttgtttgtgagctcccggtcatcaatc |
| | | atccccataatccttgttagattatcaattttaaaaaact |
| | | aacagtigtcagcctgtcccgctttaatatcatacgccgt |
| | | tatacgttgtttacgctttgaggaatcccaatggataaa |
| | | aagcctctgaa |
| SEQ ID No. 49 | hMbPylRS-NBK-mutations | caacgacaccgagctgagcaagcagatcttccgggtgg |
| | | acaaaaacctgtgcctgaggcccatgctggcccccacc |
| | | ctgattaactacgcgcggaaactggaccggatcctgcct |
| | | ggccccatcaagatcttcgaagtgggcccctgctaccgg |
| | | aaagagagcgacggcaaagagcacctggaagagttta |
| | | caatggtcaacttcgcgcagatgggcagcggctgcacc |
| | | agagagaatctggaagctctgatcaaagagttcctgga |
| | | ctatctggaaatcgacttcgagatcgtgggcg |

Table 4 provide amino acid sequences as disclosed herein.

TABLE 4
Amino Acid Sequences

| Sequence ID | Amino Acids |
|---|---|
| SEQ ID No. 50 | GGGGDDK |
| SEQ ID No. 51 | AHIVMVDAYKPTKGSGK |
| SEQ ID No. 52 | GGSGGGGSGGS |
| SEQ ID No. 53 | GGSGGGSG |
| SEQ ID No. 54 | GGSGGGSGGS |
| SEQ ID No. 55 | GGSGGGSGG |

While illustrative embodiments have been illustrated and described, it will be appreciated that various changes can be made therein without departing from the spirit and scope of the disclosure.

1. An exogenously triggerable self-assembling protein construct, comprising:
a caged reactive first protein fragment comprising a first stimulus-responsive cleavable moiety capable of cleaving from the caged reactive first protein fragment, upon application of a predetermined first stimulus, to provide a reactive first protein fragment;
a first split protein linked with the caged reactive first protein fragment;
a complementary reactive second protein fragment capable of reacting with the first reactive protein fragment; and
a second split protein linked with the complementary reactive second protein fragment,
wherein the first reactive protein fragment is adapted to react covalently with the complementary reactive second protein fragment to provide a self-assembled ligated protein or a portion thereof; and
wherein the first split protein is adapted to associate with the second split protein and to form an active protein in accordance with the reaction of the first reactive protein fragment and the complementary reactive second protein fragment providing the self-assembled protein or the portion thereof.

2. The self-assembling protein construct of claim 1, wherein the first split protein and the second split protein respectively comprise fragments of a first fluorescent protein, and wherein the active protein comprises the first fluorescent protein.

3. The self-assembling protein construct of claim 1, wherein the first split protein comprises a second fluorescent protein and the second split protein comprises a third fluorescent protein, and wherein the active protein comprises the self-assembled protein, the second fluorescent protein, and the third fluorescent protein.

4. The self-assembling protein construct of claim 2, wherein the first fluorescent protein, the second fluorescent protein, or the third fluorescent protein respectively comprises EGFP, UnaG (SEQ ID No. 41), mCherry, or mRuby.

5. The self-assembling protein construct of claim 1, wherein the first split protein and the second split protein respectively comprise inactive fragments of a luminescent protein, and wherein the active protein comprises the luminescent protein.

6. The self-assembling protein construct of claim 5, wherein the luminescent protein comprises a luciferase.

7. The self-assembling protein construct of claim 1, wherein the first split protein and the second split protein respectively comprise inactive fragments of an enzyme, and wherein the active protein comprises the enzyme.

8. The self-assembling protein construct of claim 7, wherein the enzyme is a DNA recombinase.

9. The self-assembling protein construct of claim 1, wherein the caged reactive first protein fragment, the complementary reactive second protein fragment, the first split protein, or the second split protein is coupled with a biomaterial or a biocompatible material.

10. The self-assembling protein construct of claim 9, wherein the biomaterial or the biocompatible material comprises a lipid bilayer, a hydrogel, or a cell membrane.

11. The self-assembling protein construct of claim 1, wherein the first stimulus-responsive cleavable moiety is selected from a group consisting of a photo-cleavable moiety, an enzyme-cleavable moiety, a ribozyme-cleavable moiety, a redox-cleavable moiety, an acid-cleavable moiety, a base-cleavable moiety, a nucleophile-cleavable moiety, an electrophile-cleavable moiety, an organometallic moiety having one or more chelating agents, a double-stranded DNA, a temperature-cleavable moiety, a hydrolyzable moiety, a transition metal-triggered cleavage reaction-cleavable moiety, and a cycloaddition-mediated cleavage reaction-cleavable moiety.

12. The self-assembling protein construct of claim 1, wherein the first stimulus-responsive cleavable moiety comprises a matrix metalloproteinase (MMP)-cleavable sequence; a cathepsin-cleavable sequence; an elastase-cleavable sequence; a disulfide moiety; a thioketal moiety; a nitrobenzyl moiety; a coumarin moiety; a hydrazone moiety; an oxime moiety; an acetal moiety; a silyl ether moiety; a transcyclooctene moiety; or an ester moiety.

13. The self-assembling protein construct of claim 1, wherein the first stimulus-responsive cleavable moiety is selected from Nε-(o-nitrobenzyloxycarbonyl), 2-nitrobenzyl, 3-nitrobenzyl, 4-nitrobenzyl, 2,3-dinitrobenzyl, 2,4-dinitrobenzyl, 2,6-dinitrobenzyl, 2-nitro-4,5-dimethoxybenzyl, 6-nitrobenzo[d][1,3]dioxol-5-yl, benzyl, naphthyl, anthryl, phenanthryl, pyrene, perylene, coumarin, caffeic acid chlorambucil, 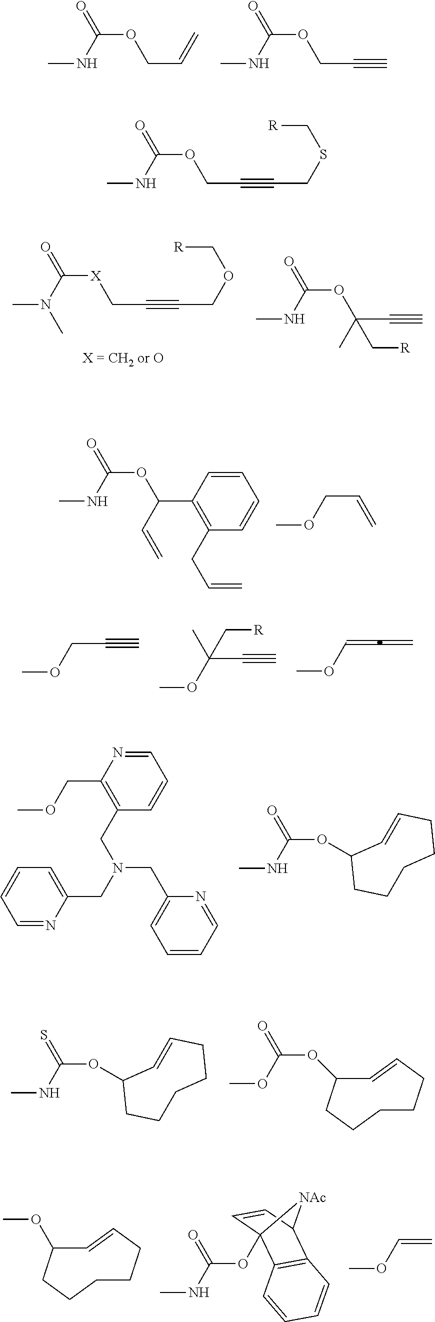 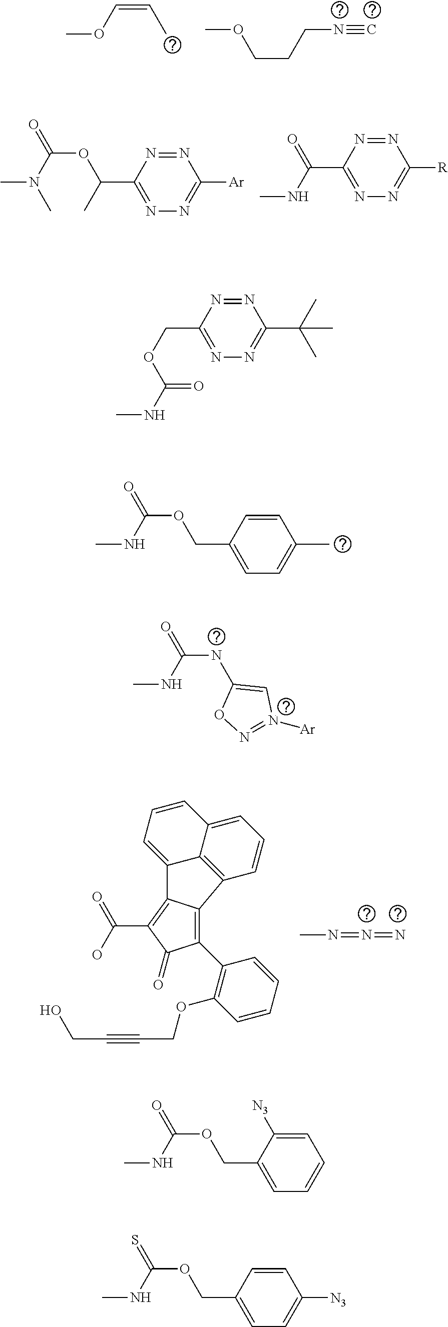 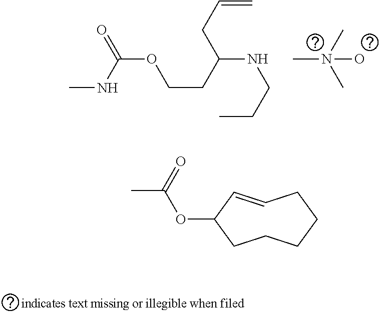
and combinations thereof.

14. The self-assembling protein construct of claim 1, wherein the reactive first protein fragment comprises a first reactive moiety; and the complementary reactive second protein fragment comprises a second reactive moiety; and the first and second reactive moieties are capable of reacting to form a covalent bond.

15. The self-assembling protein construct of claim 1, wherein the reactive first protein fragment and the complementary reactive second protein fragment respectively comprise a ligating sequence selected from a SpyCatcher sequence, a SpyCatcher002 sequence, SpyCatcher003 sequence, SpyCatcher^{DDDDK} sequence, a SpyCatcher ΔN1ΔC1 sequence, a DogCatcher sequence, a SpyStapler sequence, a SpyLigase sequence, a SnoopLigase sequence, a transglutaminase factor XIII, a sortase recognition sequence, a butelase recognition sequence, a OaAEP1b recognition sequence, a SpyTag (SEQ ID No. 7, SEQ ID No. 8) sequence, a SpyTag002 sequence, a SpyTag003 sequence, a DogTag sequence, a BDTag sequence, a KTag sequence, a SnoopTag sequence, a SdyTag sequence, a SdyCatcher sequence, or a NeissLock sequence; wherein the reactive first and second protein fragments are complementarily reactive to each other.

16. The self-assembling protein construct of claim 1, wherein:
the first split protein comprises a first portion of an UnaG (SEQ ID No. 41) fluorescent protein, the first portion comprising an N-terminus of the UnaG (SEQ ID No. 41) fluorescent protein;
the first split protein is bound to a C-terminus of the reactive first protein fragment;
the second split protein comprises a second portion of the UnaG (SEQ ID No. 41) fluorescent protein, the second portion comprising a C-terminus of the UnaG (SEQ ID No. 41) fluorescent protein; and
the second split protein is bound to an N-terminus of the complementary reactive second protein fragment.

17. The self-assembling protein construct of claim 1, wherein the caged reactive first protein fragment and the complementary reactive second protein fragment are non-functional.

18. The self-assembling protein construct of claim 1, wherein the self-assembled protein is a functional protein.

19. The self-assembling protein construct of claim 1, wherein the predetermined first stimulus is selected from: electromagnetic radiation, biocompatible electromagnetic radiation, an enzyme, a redox-active reagent (e.g., an electron donor, an electron acceptor), an acid, a base, a nucleophilic molecule, an electrophilic molecule, a chelating agent, a predetermined temperature, water, a transition metal, tetrazine, a cycloalkene, a cycloalkyne, a cyanoalkylsilane, a ketone, a phosphinyl compound, and (BPIN)2.

20. The self-assembling protein construct of claim 1, further comprising one or more additional caged reactive protein fragments and one or more complementary reactive second protein fragments.

21. The self-assembling protein construct of claim 1, wherein the complementary reactive second protein fragment is caged and comprises a second stimulus-responsive cleavable moiety capable of cleaving from the caged reactive second protein fragment upon application of a predetermined second stimulus to provide the complementary reactive second protein fragment.

22. The self-assembling protein construct of claim 21, wherein the first stimulus-responsive cleavable moiety and the second stimulus-responsive cleavable moiety are the same.

23. The self-assembling protein construct of claim 21, wherein the predetermined first stimulus and the second predetermined stimulus are the same.

24. A method of controlling protein function, comprising:
applying a predetermined stimulus to the self-assembling protein construct of claim 1 at a predetermined time and location, wherein the first reactive protein fragment and the complementary reactive second protein fragment self-assemble to provide the functional protein or a portion thereof and the active protein.

25-36. (canceled)

37. A hydrogel material, comprising:
a caged reactive first protein fragment covalently bonded to a hydrogel matrix comprising a first stimulus-responsive cleavable moiety capable of cleaving from the caged reactive first protein fragment, upon application of a predetermined first stimulus, to provide a reactive first protein fragment;
a first split protein linked with the caged reactive first protein fragment;
a complementary reactive second protein fragment that is optionally bonded to the hydrogel matrix capable of reacting with the first reactive protein fragment; and
a second split protein linked with the complementary reactive second protein fragment,
wherein the first reactive protein fragment is adapted to react with the complementary reactive second protein fragment to provide a self-assembled protein or a portion thereof; and
wherein the first split protein is adapted to associate with the second split protein and to form an active protein bonded to the hydrogel network in accordance with the reaction of the first reactive protein fragment and the complementary reactive second protein fragment providing the self-assembled protein or the portion thereof.

38-53. (canceled)


SEQUENCE LISTING (55 sequences)

SEQ ID NO: 1 - DNA, 39 bp, unidentified
  source 1..39: mol_type = other DNA
agatacgcatatggttgataccttatcaggtttatcaag

SEQ ID NO: 2 - DNA, 35 bp, unidentified
  source 1..35: mol_type = other DNA
cgtgcctcgagaatatgagcgtcacctttagttgc

SEQ ID NO: 3 - DNA, 35 bp, unidentified
  source 1..35: mol_type = other DNA
attctcatagcgtgatgaggacggcaaagagttag

SEQ ID NO: 4 - DNA, 40 bp, unidentified
  source 1..40: mol_type = other DNA
catcacgctatgagaatttaatatgggtagcactatcttc

SEQ ID NO: 5 - DNA, 39 bp, unidentified; identical to SEQ ID NO: 1
  source 1..39: mol_type = other DNA

SEQ ID NO: 6 - DNA, 53 bp, unidentified
  source 1..53: mol_type = other DNA
tacgtcagctcgaggccggtttctggaagaatatgagcgtcacctttagttgc

SEQ ID NO: 7 - DNA, 45 bp, unidentified
  source 1..45: mol_type = other DNA
agcttgcacacatagtaatggtagacgcctacaagccgacgaagc

SEQ ID NO: 8 - DNA, 45 bp, unidentified
  source 1..45: mol_type = other DNA
tcgagcttcgtcggcttgtaggcgtctaccattactatgtgtgca

SEQ ID NO: 9 - DNA, 25 bp, unidentified
  source 1..25: mol_type = other DNA
gatctcgatcccgcgaaattaatac

SEQ ID NO: 10 - DNA, 48 bp, unidentified
  source 1..48: mol_type = other DNA
gtctaccattactatgtgtgcgccaccgctcgaattagtctgcgcgtc

SEQ ID NO: 11 - DNA, 44 bp, unidentified
  source 1..44: mol_type = other DNA
gcacgaagcttatgaaaatcgaagaaggtaaactggtaatctgg

SEQ ID NO: 12 - DNA, 36 bp, unidentified
  source 1..36: mol_type = other DNA
acagtctcgaggctcgaattagtctgcgcgtctttc

SEQ ID NO: 13 - DNA, 42 bp, unidentified
  source 1..42: mol_type = other DNA
cagactaattcgagcggtggcgcacacatagtaatggtagac

SEQ ID NO: 14 - DNA, 28 bp, unidentified
  source 1..28: mol_type = other DNA
ccctatagtgagtcgtattaatttcgcg

SEQ ID NO: 15 - DNA, 58 bp, unidentified
  source 1..58: mol_type = other DNA
tagggttttgtttaactttaagaaggagatatacttacccatatggataaaaagcctc

SEQ ID NO: 16 - DNA, 45 bp, unidentified
  source 1..45: mol_type = other DNA
aaaacagccaagctggagacttacaggttagtagaaataccattg

SEQ ID NO: 17 - DNA, 43 bp, unidentified
  source 1..43: mol_type = other DNA
ttacgctttgaggaatcccaatggataaaaagcctctgaacac

SEQ ID NO: 18 - DNA, 19 bp, unidentified
  source 1..19: mol_type = other DNA
ataagcgtagcgcatcagg

SEQ ID NO: 19 - DNA, 29 bp, unidentified
  source 1..29: mol_type = other DNA
gcagtttcaaacgctaaattgcctgatgc

SEQ ID NO: 20 - DNA, 51 bp, unidentified
  source 1..51: mol_type = other DNA
atgggtaagtatatctccttcttaaagttaaacaaaaccctatagtgagtc

SEQ ID NO: 21 - DNA, 22 bp, unidentified
  source 1..22: mol_type = other DNA
actaacagttgtcagcctgtcc

SEQ ID NO: 22 - DNA, 19 bp, unidentified
  source 1..19: mol_type = other DNA
ataagcgtagcgcatcagg

SEQ ID NO: 23 - DNA, 28 bp, Methanosarcina bakeri
  source 1..28: mol_type = genomic DNA
caccagagagaatctggaagctctgatc

SEQ ID NO: 24 - DNA, 23 bp, Methanosarcina bakeri
  source 1..23: mol_type = genomic DNA
gcctcaggcacaggtttttgtcc

SEQ ID NO: 25 - DNA, 369 bp, unidentified
  source 1..369: mol_type = other DNA
atggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattctcgagcaccaccaccaccaccactga

SEQ ID NO: 26 - DNA, 369 bp, unidentified
  source 1..369: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattctcgagcaccaccaccaccaccactga

SEQ ID NO: 27 - DNA, 384 bp, unidentified
  source 1..384: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattcttccagaaaccggcctcgagcaccaccaccaccaccactga

SEQ ID NO: 28 - DNA, 765 bp, unidentified
  source 1..765: mol_type = other DNA
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccggtggttctggtggttctggtggttctggtggttctggtggttctgctcacatcgttatggttgacgcttacaaaccgaccaaatga

SEQ ID NO: 29 - DNA, 783 bp, unidentified
  source 1..783: mol_type = other DNA
atggtttctaaaggtgaagaactgatcaaagaaaacatgcgtatgaaagttgttatggaaggttctgttaacggtcaccagttcaaatgcaccggtgaaggtgaaggtcgtccgtacgaaggtgttcagaccatgcgtatcaaagttatcgaaggtggtccgctgccgttcgctttcgacatcctggctacctctttcatgtacggttctcgtaccttcatcaaatacccggctgacatcccggacttcttcaaacagtctttcccggaaggtttcacctgggaacgtgttacccgttacgaagacggtggtgttgttaccgttacccaggacacctctctggaagacggtgaactggtttacaacgttaaagttcgtggtgttaacttcccgtctaacggtccggttatgcagaaaaaaaccaaaggttgggaaccgaacaccgaaatgatgtacccggctgacggtggtctgcgtggttacaccgacatcgctctgaaagttgacggtggtggtcacctgcactgcaacttcgttaccacctaccgttctaaaaaaaccgttggtaacatcaaaatgccgggtgttcacgctgttgaccaccgtctggaacgtatcgaagaatctgacaacgaaacctacgttgttcagcgtgaagttgctgttgctaaatactctaacctgggtggtggtatggacgaactgtacaaaaagcttgcacacatagtaatggtagacgcctacaagccgacgaagctcgagcaccaccaccaccaccactga

SEQ ID NO: 30 - DNA, 471 bp, unidentified
  source 1..471: mol_type = other DNA
atgcaagctaaacctcaaattccgaaagataaatcaaaagtggcaggctatattgaaattccagatgctgatattaaagaaccagtatatccaggaccagcaacacgcgaacaattaaatagaggtgtaagctttgcaaaagaaaatcaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagaaacgttaagccaacagctgtagaagttctagatgaacaaaaaggtaaagataaacaattaacattaattacttgtgatgattacaatgaagagacaggcgtttgggaaacacgtaaaatctttgtagctacagaagtcaaactcgagcaccaccaccaccaccactga

SEQ ID NO: 31 - DNA, 1464 bp, unidentified
  source 1..1464: mol_type = other DNA
atggttgaaaaatttgtgggtacgtggaaaattgcggattcgcataattttggcgaatacctgaaagctatcggggcacctaaagaactgagtgacggaggcgatgccacgaccccaactctttatatttcccagaaggacggtgataaaatgaccgtaaagatcgagaacggcccgcccaccttcttagatacacaagtcaaatttaaattgggtgaagaattcgatgagttcccgagcgaccgtcgcaaaggtggctctggaggtggatctggcggttctgcacacatagtaatggtagacgcctacaagccgacgaagaagcttatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagcctcgagcaccaccaccaccaccactga

SEQ ID NO: 32 - DNA, 1470 bp, unidentified
  source 1..1470: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagcggtggcgcacacatagtaatggtagacgcctacaagccgacgaagggtggctctggaggtggatctggcatggttgaaaaatttgtgggtacgtggaaaattgcggattcgcataattttggcgaatacctgaaagctatcggggcacctaaagaactgagtgacggaggcgatgccacgaccccaactctttatatttcccagaaggacggtgataaaatgaccgtaaagatcgagaacggcccgcccaccttcttagatacacaagtcaaatttaaattgggtgaagaattcgatgagttcccgagcgaccgtcgcaaaaagcttggtaccctcgagcaccaccaccaccaccactga

SEQ ID NO: 33 - DNA, 1383 bp, unidentified
  source 1..1383: mol_type = other DNA
atgtccggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaggtggctctggaggtggatctggcggttctgcacacatagtaatggtagacgcctacaagccgacgaagaagcttatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagcctcgagcaccaccaccaccaccactga

SEQ ID NO: 34 - DNA, 1384 bp, unidentified
  source 1..1384: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagcggtggcgcacacatagtaatggtagacgcctacaagccgacgaagggtggcagtggtggtgggagcggtcggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaaagcttggtaccctcgagcaccaccaccaccaccactga

SEQ ID NO: 35 - DNA, 654 bp, unidentified
  source 1..654: mol_type = other DNA
atggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggctctggaggtggatctggcatggttgaaaaatttgtgggtacgtggaaaattgcggattcgcataattttggcgaatacctgaaagctatcggggcacctaaagaactgagtgacggaggcgatgccacgaccccaactctttatatttcccagaaggacggtgataaaatgaccgtaaagatcgagaacggcccgcccaccttcttagatacacaagtcaaatttaaattgggtgaagaattcgatgagttcccgagcgaccgtcgcaaaggtggcagtctcgagcaccaccaccaccaccactga

SEQ ID NO: 36 - DNA, 663 bp, unidentified
  source 1..663: mol_type = other DNA
atggttgaaaaatttgtgggtacgtggaaaattgcggattcgcataattttggcgaatacctgaaagctatcggggcacctaaagaactgagtgacggaggcgatgccacgaccccaactctttatatttcccagaaggacggtgataaaatgaccgtaaagatcgagaacggcccgcccaccttcttagatacacaagtcaaatttaaattgggtgaagaattcgatgagttcccgagcgaccgtcgcaaaggtggctctggaggtggatctggcggttctatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattaagcttggtaccctcgagcaccaccaccaccaccactga

SEQ ID NO: 37 - DNA, 570 bp, unidentified
  source 1..570: mol_type = other DNA
atggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggcagtggtggtgggagcggcggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaaagcttggtaccctcgagcaccaccaccaccaccactga

SEQ ID NO: 38 - DNA, 582 bp, unidentified
  source 1..582: mol_type = other DNA
atgtccggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaggtggctctggaggtggatctggcggttctatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattaagcttggtaccctcgagcaccaccaccaccaccactga

SEQ ID NO: 39 - DNA, 570 bp, unidentified
  source 1..570: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggcagtggtggtgggagcggcggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaaagcttggtaccctcgagcaccaccaccaccaccactga

SEQ ID NO: 40 - DNA, 573 bp, unidentified
  source 1..573: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggcagtggtggtgggagcggcggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaacttccagaaaccggcctcgagcaccaccaccaccaccactga

SEQ ID NO: 41 - DNA, 435 bp, unidentified
  source 1..435: mol_type = other DNA
atggtggaaaagttcgttggtacctggaaaatcgctgatagccataattttggtgaatatctgaaggcgattggcgctccgaaagaactgagcgatggtggcgacgctaccaccccgaccctgtacatcagccagaaagatggtgacaagatgaccgtgaaaattgaaaatggtccgccgaccttcctggatacccaagttaaatttaagctgggtgaagaattcgatgaatttccgagcgaccgtcgtaaaggtgtgaagagcgtggttaacctggttggcgaaaagctggtgtatgttcagaagtgggacggcaaagaaaccacctacgtgcgtgaaatcaaagatggtaaactggtggtgaccctgaccatgggcgacgtggttgcagttcgtagctatcgtcgtgcgaccgaacatcaccatcaccatcac

SEQ ID NO: 42 - DNA, 447 bp, unidentified
  source 1..447: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggcagtggtggtgggagcggcgtgactggatacagactttttgaagagatcctggaattcgggggtggaggttcactcgagcaccaccaccaccaccactga

SEQ ID NO: 43 - DNA, 573 bp, unidentified
  source 1..573: mol_type = other DNA
atggtcttcacactcgaagatttcgttggggactgggaacagacagccgcgtacaacctggaccaagtccttgaacagggaggtgtgtccagtttgctgcagaatctcgcggtgtccgtaactccgatccaaaggattgtccgcagcggtgaaaatgcgctgaagatcgacatccatgtcatcatcccgtatgaaggtctgagcgcggaccaaatggcacagatcgaagaagtgtttaaggtggtgtaccctgtggatgatcatcactttaaggtgatcctgccgtatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggtggctctggaggtggatctggcggttctgcacacatagtaatggtagacgcctacaagccgacgaagctcgagcaccaccaccaccaccactga

SEQ ID NO: 44 - DNA, 1698 bp, unidentified
  source 1..1698: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggctctggaggtggatctggcggtgttaaatccgtagtcaacttggtcggagaaaaactcgtatatgtccaaaaatgggatggcaaagagacaacctacgtgcgtgaaattaaagacgggaaactggttgtgacgctgacaatgggcgacgtggtggcagtgcgctcgtaccggcgtgcaaccgaaggaagcggtgccaccaatttctccctcctgaagcaggctggggacgtggaggaaaaccctggccccatggttgaaaaatttgttggtacgtggaaaatagcggattctcataattttggcgaatatctgaaggctatcggggcgcctaaagagctgagtgatggaggcgatgccacgactccaactctgtatatttcacagaaggacggtgataaaatgaccgttaagatcgagaatggcccgcccaccttcctggatacacaggtaaagtttaaacttggtgaagaatttgatgagttcccgagcgacagacgcaaaggaggctctggtggaggctctggaggtggctctggagcacacatagtaatggtagacgcctacaagccgacgaagggaagcggagctactaacttcagcctgctgaagcaggctggagacgtggaggagaaccctggacctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 45 - DNA, 1518 bp, unidentified
  source 1..1518: mol_type = other DNA
atggccacctctgatgaagtcaggaagaacctgatggacatgttcagggacaggcaggccttctctgaacacacctggaagatgctcctgtctgtgtgcagatcctgggctgcctggtgcaagctgaacagcggcggcagcgggagtacagcacacatagtaatggtagacgcctacaagccgacgaaggtgcccaagaagaagaggaaagtcggaagcggtgccaccaatttctccctcctgaagcaggctggggacgtggaggaaaaccctggccccgtgcccaagaagaagaggaaagtcggttccatggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatttctggtggcagtggaagcactaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggat

SEQ ID NO: 46 - DNA, 1275 bp, unidentified
  source 1..1275: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggggggaagtggagggagtggggggagcggggcacacatagtaatggtagacgcctacaagccgacgaagggaagcggagctactaacttcagcctgctgaagcaggctggagacgtggaggagaaccctggacctatggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggtggatctggaggtggatctggaggtatgagcaaagatggcaaaaagaagaaaaagaagtcaaagacaaagtgtgtaattatg

SEQ ID NO: 47 - DNA, 2010 bp, unidentified
  source 1..2010: mol_type = other DNA
atggttgataccttatcaggtttatcaagtgagcaaggtcagtccggtgatatgacaattgaagaagatagtgctacccatattaaattctcatagcgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattgggggaagtggagggagtggggggagcggggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagctactaacttcagcctgctgaagcaggctggagacgtggaggagaaccctggacctgcacacatagtaatggtagacgcctacaagccgacgaaggggggaagtggagggagtggggggagcggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggtggatctggaggtggatctggaggtatgagcaaagatggcaaaaagaagaaaaagaagtcaaagacaaagtgtgtaattatg

SEQ ID NO: 48 - DNA, 440 bp, unidentified
  source 1..440: mol_type = other DNA
gtatttctactaacctgtaagtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtgagctcccggtcatcaatcatccccataatccttgttagattatcaattttaaaaaactaacagttgtcagcctgtcccgctttaatatcatacgccgttatacgttgtttacgctttgaggaatcccaatggataaaaagcctctgaa

SEQ ID NO: 49 - DNA, 299 bp, Methanosarcina bakeri
  source 1..299: mol_type = genomic DNA
caacgacaccgagctgagcaagcagatcttccgggtggacaaaaacctgtgcctgaggcccatgctggcccccaccctgattaactacgcgcggaaactggaccggatcctgcctggccccatcaagatcttcgaagtgggcccctgctaccggaaagagagcgacggcaaagagcacctggaagagtttacaatggtcaacttcgcgcagatgggcagcggctgcaccagagagaatctggaagctctgatcaaagagttcctggactatctggaaatcgacttcgagatcgtgggcg

SEQ ID NO: 50 - protein, 7 aa, synthetic construct
GGGGDDK

SEQ ID NO: 51 - protein, 17 aa, synthetic construct
AHIVMVDAYKPTKGSGK

SEQ ID NO: 52 - protein, 11 aa, synthetic construct
GGSGGGGSGGS

SEQ ID NO: 53 - protein, 8 aa, synthetic construct
GGSGGGSG

SEQ ID NO: 54 - protein, 10 aa, synthetic construct
GGSGGGSGGS

SEQ ID NO: 55 - protein, 9 aa, synthetic construct
GGSGGGSGG